(19) United States
(12) Patent Application Publication
(10) Pub. No.: US 2016/0022480 A1
(43) Pub. Date: Jan. 28, 2016

(54) THERMAL EYE COMPRESS SYSTEMS AND METHODS OF USE

(71) Applicants: Seth A. BISER, (US); Gina BARNABA, (US); Joshua STEWART, (US)

(72) Inventors: BISER A. SETH, Fleetwood, NY (US); Gina BARNABA, Scarsdale, NY (US); Joshua STEWART, Seattle, WA (US)

(21) Appl. No.: 14/775,534

(22) PCT Filed: Mar. 14, 2014
(86) PCT No.: PCT/US14/28482

§ 371 Date: Sep. 11, 2015

Related U.S. Application Data
(63) Continuation-in-part of application No. 14/179,290, filed on Feb. 12, 2014.
(60) Provisional application No. 61/852,263, filed on Mar. 15, 2013.
(60) Provisional application No. 61/962,067, filed on Oct. 30, 2013.

(51) Int. Cl. A61F 7/02

(52) U.S. Cl. CPC A61F 7/02; A61F 2007/0004

(57) ABSTRACT

An apparatus includes a flexible frame, a coupling portion, and an insulation portion. The flexible frame is configured to support a thermal therapeutic member and is transitioned between a first configuration and a second configuration to place the therapeutic member in substantially continuous contact with a target portion of the body. The coupling portion is coupled to the flexible frame and is transitioned from a first configuration to a second configuration to retain the flexible frame in its second configuration. The insulation portion is transitioned from a first configuration to a second configuration to be coupled to the flexible frame. The insulation portion at least partially controls a transfer of thermal energy between the thermal therapeutic member and the target portion of the body.

### CROSS-REFERENCE TO RELATED APPLICATIONS

This application is a continuation-in-part of U.S. patent application Ser. No. 14/179,290 entitled, “Thermal Eye Compress Systems and Methods of Use,” filed Feb. 12, 2014, which claims priority to and the benefit of U.S. Provisional Patent Application Ser. No. 61/852,263 entitled, “Eye Compress Cover and Method of Use,” filed Mar. 15, 2013 and U.S. Patent Application Ser. No. 61/962,067 entitled, “Moistened Nonwoven Disposable Folded Sheets for Use on an Eye Compress, and Methods for Using Same,” filed Oct. 30, 2013, the disclosures of which are incorporated herein by reference in their entireties. This application claims priority to U.S. Provisional Patent Application Ser. No. 61/852,263 entitled, “Eye Compress Cover and Method of Use,” filed Mar. 15, 2013. This application also claims priority to U.S. Patent Application Ser. No. 61/962,067 entitled, “Moistened Nonwoven Disposable Folded Sheets for Use on an Eye Compress, and Methods for Using Same,” filed Oct. 30, 2013.

### BACKGROUND

The embodiments described herein relate generally to apparatus, systems, and methods for applying a therapeutic member to a portion of a user's body. More particularly, the embodiments described herein relate to compress devices, systems, kits, and/or methods for providing therapeutic benefit to the eye region of a user by the application or removal of thermal energy, and/or by the application of a therapeutic member.

The application of hot and/or cold compresses is a known therapeutic treatment for some physical ailments. In some instances, a method of thermal compress therapy includes a user holding a cloth (e.g., a washcloth) under hot or cold running water, or in a basin of hot or cold water, and then applying the moist, temperature-adjusted cloth to the desired body part. In some such instances, the cloth is maintained in contact with the desired body part through manual intervention (e.g., the user holds the cloth in place). In some instances, a user may not be able to maintain the position of the cloth due to an ailment and/or impairment (e.g., arthritis or the like).

In other instances, a thermal pack can be heated or cooled, and applied against a body part of the user. Some such thermal packs can be filled with gelatinous substances that can conform to the anatomy under gentle pressure. For example, in some instances, a thermal pack can be used to apply thermal energy to or remove thermal energy from the eye region of the user. In some embodiments, the thermal packs can be included in a device that includes a frame configured to support the thermal packs and a strap system configured to retain the thermal packs in a fixed position relative to the eye region of the user. The anatomy of the eye region, however, can result in challenges to the application of thermal packs. For example, the contour of the eye region can result in challenges to placing the thermal packs in contact with the eye region with a relatively consistent and comfortable amount of force. As such, the level of discomfort and/or ineffective application or removal of thermal energy can, in some instances, deter a user from using some such devices.

Thus, a need exists for improved compress devices, systems, kits, and methods for providing therapeutic benefit to, for example, sensitive portions of the body such as the eye region, by the application or removal of thermal energy and/or by the application of a therapeutic member.

### SUMMARY

Apparatus, systems, and methods for providing therapeutic benefit to the eye region of a user by the application or removal of thermal energy and/or by the application of a therapeutic member are described herein. In some embodiments, an apparatus includes a flexible frame, a coupling portion, and an insulation portion. The flexible frame is configured to support a thermal therapeutic member and is transitioned between a first configuration and a second configuration to place the therapeutic member in substantially continuous contact with a target portion of the body. The coupling portion is coupled to the flexible frame and is transitioned from a first configuration to a second configuration to retain the flexible frame in its second configuration. The insulation portion is transitioned from a first configuration to a second configuration to be coupled to the flexible frame. The insulation portion at least partially controls a transfer of thermal energy between the thermal therapeutic member and the target portion of the body.

### BRIEF DESCRIPTION OF THE DRAWINGS

FIG. 1 is a schematic illustration of a therapeutic device according to an embodiment.

FIGS. 2 and 3 are a front view and a side view, respectively, of a therapeutic device coupled to a portion of the body according to another embodiment.

FIG. 4 is a rear view of a portion of the therapeutic device of FIGS. 2 and 3 and a therapeutic member.

FIG. 5 is an enlarged view of a portion of a therapeutic member included in the therapeutic device of FIGS. 2 and 3, identified as the region 5 in FIG. 4.

FIG. 6 is an exploded view of a coupler included in the therapeutic member of FIG. 5.

FIGS. 7 and 8 are a front view and a rear view, respectively, of a flexible frame included in the therapeutic device of FIGS. 2 and 3 in a first configuration.

FIGS. 9-11 are a front view, a perspective view, and a side view, respectively, of a portion of the therapeutic device of FIGS. 2 and 3 coupled to the portion of the body and illustrating the flexible frame in a second configuration.

FIG. 12 is a rear view of a portion of the therapeutic device of FIGS. 2 and 3 illustrating a coupling portion.

FIG. 13 is a schematic illustration of a strap included in the coupling portion of FIG. 12.

FIG. 14 is a schematic illustration of a coupling member included in the coupling portion of FIG. 12.

FIG. 15 is a schematic illustration of a closure member included in the coupling portion of FIG. 12.

FIG. 16 is a schematic illustration of the strap of FIG. 12 partially coupled to the coupling member of FIG. 14.

FIGS. 17 and 18 are perspective views of a portion of the strap illustrated in FIG. 13 in a first configuration and a second configuration, respectively.

FIG. 19 is a schematic illustration of the strap of FIG. 13 in a third configuration.

FIGS. 20 and 21 are a rear perspective view and a front perspective view, respectively, of the portion of the strap illustrated in FIGS. 17 and 18 in the third configuration.

FIG. 22 is a front view of a first insulating member included in the therapeutic device of FIGS. 2 and 3.

FIG. 23 is a front view of the flexible frame of FIG. 7.

FIG. 24 is a side view of a first coupling member and a second coupling member included in the flexible frame of FIG. 23.

FIGS. 25 and 26 are a front view and a bottom view, respectively, of the first insulating member of FIG. 22 in a first configuration and partially coupled to the flexible frame of FIG. 7.

FIGS. 27 and 28 are a front view and a bottom view, respectively, of the first insulating member of FIG. 22 in a second configuration and coupled to the flexible frame of FIG. 7.

FIGS. 29 and 30 are a front view and a side view, respectively, of the therapeutic device of FIGS. 2 and 3 coupled to the portion of the body.

FIG. 31 is a rear view of the therapeutic device of FIGS. 2 and 3 and a second insulating member according to an embodiment.

FIG. 32 is a rear view of the second insulating member of FIG. 31 coupled to the therapeutic device of FIGS. 2 and 3.

FIG. 33 is a side view of the second insulating member of FIG. 31.

FIG. 34 is a rear view of a user spraying a fluid on the second insulating member of FIG. 21 while coupled to the therapeutic device of FIGS. 2 and 3.

FIG. 35 is a top view of a package configured to house one or more of the second insulating members of FIG. 21 according to an embodiment.

FIGS. 36 and 37 are illustrations of a method for transferring thermal energy to the therapeutic member included in the therapeutic device of FIGS. 2 and 3.

FIGS. 38 and 39 are illustrations of a method for coupling the therapeutic device of FIGS. 2 and 3 to the head of a user.

FIGS. 40 and 41 are illustrations of a method for increasing a thermal output and a compressive force of the therapeutic member when the therapeutic device of FIGS. 2 and 3 is coupled to the head of the user.

FIG. 42 is an illustration of a method for removing the second insulating member of FIG. 21 from the therapeutic device of FIGS. 2 and 3 when the therapeutic device is coupled to the head of the user.

FIG. 43 is a perspective view of a therapeutic device coupled to a portion of the body according to another embodiment.

FIGS. 44-46 are a front perspective view, a front view, and a bottom perspective view, respectively, of a flexible frame included in a therapeutic device according to another embodiment.

FIG. 47 is a rear perspective view of the flexible frame of FIGS. 44-46 coupled to a therapeutic member.

FIGS. 48 and 49 are a front perspective view and a rear perspective view, respectively, of a flexible frame included in a therapeutic device according to another embodiment.

FIG. 50 is a rear view of the therapeutic device including the flexible frame of FIGS. 48 and 49 and a therapeutic member.

FIG. 51 is a perspective view of the therapeutic device of FIG. 50 coupled to a portion of the body.

FIG. 52 is a front view of a second insulating member according to an embodiment.

FIG. 53 is a front view of the second insulating member of FIG. 52 coupled to a flexible frame, according to an embodiment.

FIG. 54 is a front view of a second insulating member according to an embodiment.

FIG. 55 is a rear view of a second insulating member coupled to a therapeutic member, according to an embodiment.

FIG. 56 is a front view of a heating member coupled to a therapeutic member according to an embodiment.

FIG. 57 is a rear view of a heating member coupled to a therapeutic device according to another embodiment.

FIG. 58 is a front view of a heating device according to an embodiment.

FIG. 59 is a front view of a therapeutic member disposed in the heating device of FIG. 58.

FIGS. 60-62 are perspective views of a heating device according to an embodiment.

FIG. 63 is a perspective view of a hanger of a heating device according to an embodiment.

FIG. 64 is a top view of a shipping package configured to receive a therapeutic device in a first configuration, according to an embodiment.

FIG. 65 is a top view of a therapeutic device disposed in the shipping package of FIG. 64.

FIGS. 66 and 67 are a top view and a perspective view, respectively, of the shipping package of FIG. 64 in a second configuration.

FIGS. 68 and 69 are perspective views of a case configured to receive a therapeutic device in an open configuration and a closed configuration, respectively, according to an embodiment.

FIG. 70 is a top view of the shipping package of FIGS. 64-67 disposed in the case of FIGS. 68 and 69.

FIG. 71 is a cross-sectional schematic view of a portion of the therapeutic device of FIGS. 2 and 3 disposed adjacent to the face of a user.

FIG. 72 is a cross-sectional schematic view of a portion of the therapeutic devices of FIG. 43 or FIG. 48 disposed adjacent to the face of a user.

FIG. 73 is a cross-sectional schematic view of a portion of a therapeutic device disposed adjacent to the face of a user according to another embodiment.

FIG. 74 is a flowchart illustrating a method of using a sheet on a therapeutic device according to an embodiment.

### DETAILED DESCRIPTION

In some embodiments, an apparatus includes a flexible frame, a coupling portion, and an insulating portion. The flexible frame is configured to support a thermal therapeutic member and is transitioned between a first configuration and a second configuration to place the therapeutic member in substantially continuous contact with a target portion of the body. The coupling portion is coupled to the flexible frame and is transitioned from a first configuration to a second configuration to retain the flexible frame in its second configuration. The insulating portion is transitioned from a first configuration to a second configuration to be coupled to the flexible frame. The insulating portion at least partially controls a transfer of thermal energy between the thermal therapeutic member and the target portion of the body.

The embodiments described herein can include and/or can form at least a portion of a therapeutic device (e.g., a thermal compress device, system, and/or kit) that can be used to treat or alleviate a variety of abnormal physiological conditions in users, or to provide therapeutic benefit to users who are otherwise in normal condition. The devices and methods can be applied to various body parts such as, for example, soft tissues, muscles, bones, and other tissues and organs of a user. Although the embodiments and methods are described herein as being associated with and/or applied to, for example, an ocular region of a user, in other instances, the embodiments and methods can be associated with and/or applied to any suitable part of the anatomy. Accordingly, the embodiments described herein are not to be construed as being limited only to use in treatments of the eye or ocular region of a user. The embodiments can be adapted to any use in which thermal or other surface treatment is to be provided by contacting a body part of a user with a portion of a device that can transfer thermal energy to or receive thermal energy from the body part, and/or that can transfer a therapeutic member other than thermal energy to the body part.

As used in this specification, the singular forms “a,” “an” and “the” include plural referents unless the context clearly dictates otherwise. Thus, for example, the term “a member” is intended to mean a single member or a combination of members, “a material” is intended to mean one or more materials, or a combination thereof.

As used herein, the terms “about” and “approximately” generally mean plus or minus 10% of the value stated. For example, about 0.5 would include 0.45 and 0.55, about 10 would include 9 to 11, about 1000 would include 900 to 1100. In addition, the terms about and approximately can be context specific. For example, if a function is being performed, the term about when used in the context of the function so defined can include any or all variations of the function while still performing that function.

As used herein, the term “set” can refer to multiple features or a singular feature with multiple parts. For example, when referring to a set of walls, the set of walls can be considered as one wall with multiple portions, or the set of walls can be considered as multiple, distinct walls. Thus, a monolithically constructed item can include a set of walls. Such a set of walls may include multiple portions that are either continuous or discontinuous from each other. A set of walls can also be fabricated from multiple items that are produced separately and are later joined together (e.g., via a weld, an adhesive, or any suitable method).

As used herein, the term “stiffness” is related to an object's resistance to deflection, deformation, and/or displacement that is produced by an applied force, and is generally understood to be the opposite of the object's “flexibility.” For example, a wall with greater stiffness is more resistant to deflection, deformation, and/or displacement when exposed to a force than a wall having a lower stiffness. Similarly stated, an object having a higher stiffness can be characterized as being more rigid than an object having a lower stiffness. Stiffness can be characterized in terms of the amount of force applied to the object and the resulting distance through which a first portion of the object deflects, deforms, and/or displaces with respect to a second portion of the object. When characterizing the stiffness of an object, the deflected distance may be measured as the deflection of a portion of the object different from the portion of the object to which the force is directly applied. Said another way, in some objects, the point of deflection is distinct from the point where force is applied.

Stiffness (and therefore, flexibility) is an extensive property of the object being described, and thus is dependent upon the material from which the object is formed as well as certain physical characteristics of the object (e.g., cross-sectional shape, length, boundary conditions, etc.). For example, the stiffness of an object can be increased or decreased by selectively including in the object a material having a desired modulus of elasticity, flexural modulus, and/or hardness. The modulus of elasticity is an intensive property of (i.e., is intrinsic to) the constituent material and describes an object's tendency to elastically (i.e., non-permanently) deform in response to an applied force. A material having a high modulus of elasticity will not deflect as much as a material having a low modulus of elasticity in the presence of an equally applied stress. Thus, the stiffness of the object can be decreased, for example, by introducing into the object and/or constructing the object of a material having a relatively low modulus of elasticity.

In another example, the stiffness of the object can be increased or decreased by changing the flexural modulus of a material of which the object is constructed. Flexural modulus is used to describe the ratio of the applied stress on an object in flexure to the corresponding strain in the outermost portions of the object. The flexural modulus, rather than the modulus of elasticity, is used to characterize certain materials, for example plastics, that do not have material properties that are substantially linear over a range of conditions. An object with a first flexural modulus is less elastic and has a greater strain on the outermost portions of the object than an object with a second flexural modulus lower than the first flexural modulus. Thus, the stiffness of an object can be increased by including in the object a material having a high flexural modulus.

The stiffness of an object can also be increased or decreased by changing a physical characteristic of the object, such as the shape or cross-sectional area of the object. For example, an object having a length and a cross-sectional area may have a greater stiffness than an object having an identical length but a smaller cross-sectional area. As another example, the stiffness of an object can be reduced by including one or more stress concentration risers (or discontinuous boundaries) that cause deformation to occur under a lower stress and/or at a particular location of the object. Thus, the stiffness of the object can be decreased by decreasing and/or changing the shape of the object.

As used herein, certain components and/or aspects of the embodiments can be identified and/or located with the adjectives “top,” “upper,” “bottom,” “lower,” “left,” “right,” “front,” “rear,” etc. These adjectives are provided in the context of use of a compress system in therapy of the eye region of a user, and in the context of the orientation of the drawings that show an upright human user. In this context, the terms “top,” “bottom,” “left,” “right,” “front,” and “back” refer to the orientation of the compress system in relation to the user, in an applied position on the user's face when the user is standing upright (a position known as the “anatomical position”) unless explicitly stated otherwise. The compress systems disclosed herein can be worn by a user in any number of positions, including, for example, an upright (sitting or standing) or recumbent position.

Relationships and orientations associated with the components of the embodiments herein may also be described in ways that are common in anatomic medical terminology. For example, the terms “lateral” and “temporal” are used to indicate a location substantially at or along a side portion of the body, while the terms “medial” and “nasal” are used to indicate a location substantially at or along a longitudinal centerline of the body. The terms “superior” and “inferior” can be used to describe an upper or top location and a lower or bottom location, respectively, relative to the body. Similarly, the terms “anterior” and “posterior” can be used to describe a forward location or rearward location, respectively, relative to the body. Moreover, when used in reference to positions on or along the embodiments described herein, the terms “peripheral,” “outer,” and/or “distal” can refer to a position that is disposed at, along and/or relatively closer to a perimeter of the embodiment, while the terms “central,” “inner,” and/or “proximal” can refer to a position that is disposed at, along, and/or relatively closer to a center of the embodiment.

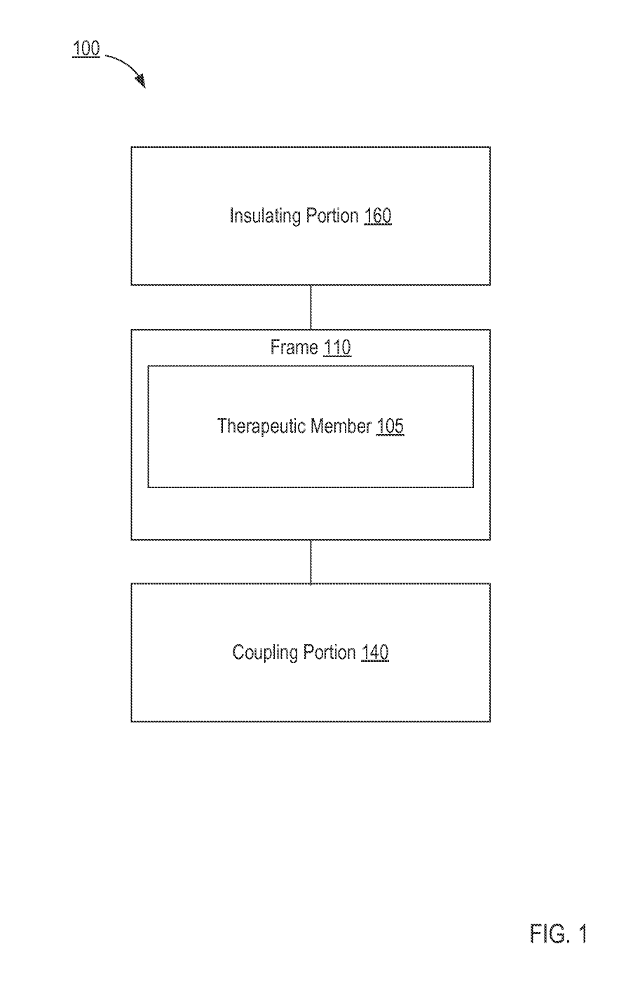

FIG. 1 is a schematic illustration of a therapeutic device 100 according to an embodiment. The therapeutic device 100 (also referred to herein as “eye compress device” or “eye compress system”) can be used to place a therapeutic member in contact with a target region of the body of a user such as, for example, the ocular region of the face of the user. The therapeutic device 100 includes a flexible frame 110, a coupling portion 140, and an insulating portion 160. The flexible frame 110 is configured to support and/or otherwise be coupled to a therapeutic member 105. The therapeutic member 105 (also referred to herein as “thermally-adjustable therapeutic member”) can be any suitable configuration. For example, in some embodiments, the therapeutic member 105 can be a thermal gel pack or the like. In such embodiments, the user can place a surface of the thermal gel pack in contact with, for example, the ocular region of the face and in turn, the thermal gel pack can transfer thermal energy to or receive thermal energy from the ocular region. More specifically, the therapeutic member 105 can include a substantially viscous material or combination of materials that can be heated or cooled to provide thermal energy to or receive thermal energy from, respectively, the body.

In some embodiments, the therapeutic member 105 can include a flexible pouch or the like that can deform when exposed to an external force. Thus, when the therapeutic member 105 is placed in contact with, for example, the ocular region of the patient, the therapeutic member 105 can elastically deform (e.g., nonpermanently deform), bend, flex, or otherwise reconfigure in such a manner that a surface area in contact with the ocular region is greater than a surface area of a substantially rigid or inflexible therapeutic member that is placed in contact with the ocular region. In some embodiments, the therapeutic member 105 can define a single inner volume such that when the therapeutic member 105 is placed in contact with the ocular region, a single volume of thermal gel can transfer thermal energy to or receive thermal energy from at least a portion of the ocular region being treated. For example, in some embodiments, the therapeutic member 105 can define a single inner volume such that when the therapeutic member 105 is placed in contact with the ocular region, a single volume of thermal gel can transfer thermal energy to or receive thermal energy from both the left eye and the right eye of the ocular region. Similarly stated, the therapeutic member 105 can be arranged such that a volume of thermal gel that transfers thermal energy to and/or receives thermal energy from the left eye of the user is in fluid communication with a volume of thermal gel that transfers thermal energy to and/or receives thermal energy from the right eye of the user.

The flexible frame 110 (also referred to herein as “frame”) is coupled to the coupling portion 140 and the insulating portion 160 and supports and/or is at least temporarily coupled to the therapeutic member 105. For example, in some embodiments, the frame 110 can include and/or define a set of snaps, buttons, protrusions, apertures, surfaces, etc. that can be matingly coupled to a corresponding set of snaps, buttons, protrusions, apertures, surfaces, etc. included in and/or defined by the therapeutic member 105. Specifically, in some embodiments, the frame 110 can include a set of snaps that can matingly engage a corresponding set of snaps included in the therapeutic member 105 to removably couple the therapeutic member 105 to the frame 110.

The frame 110 can be any suitable shape, size, or configuration. For example, in some embodiments, the flexible frame 110 can have a size and shape that is associated with at least a portion of the ocular region of the user. In such embodiments, the frame 110 can include a first lobe and a second lobe that can substantially correspond to a first eye and a second eye of the user. Thus, when the therapeutic member 105 is coupled to the frame 110 and the therapeutic member 105 is placed in contact with the ocular region of the user, the frame 110 can support the therapeutic member 105 to maintain a surface of the therapeutic member 105 in contact with, for example, the left eye region and the right eye region of the user. Moreover, in some embodiments, the first lobe and the second lobe can each define an aperture that can reduce the stiffness of the frame 110 and/or that can provide a portion of the frame 110 which can provide other means for enhancing the experience of the user, when the therapeutic device 100 is disposed about the ocular region. In some embodiments, for example, a portion of the therapeutic member 105 can be configured to extend anteriorly through the apertures such that when the therapeutic member 105 is placed in contact with the ocular region of the user, a force exerted by the therapeutic member 105 directly on the globe of the eye (and/or eye lid) is reduced. In other embodiments, the frame 110 can be substantially solid (e.g., the frame 110 does not define the apertures). Although the frame 110 is described above as supporting the therapeutic member 105 that is in contact with both the left eye and the right eye, in other embodiments, the frame 110 can support a therapeutic member that is in contact with a single eye (i.e., the left eye or the right eye) of the user.

The flexible frame 110 can be formed from any suitable material such as one or more polymers (e.g., plastics). Examples of suitable polymers can include polylactides, polyglycolides, polylactide-co-glycolides (PLGA), polyanhydrides, polyorthoesters, polyetheresters, polycaprolactones, polyesteramides, poly(butyric acid), poly(valeric acid), polyurethanes, nylons, polyesters, polycarbonates, polyacrylates, polymers of ethylene-vinyl acetates and other acyl substituted cellulose acetates, non-degradable polyurethanes, polystyrenes, polyvinyl chloride, polyvinyl fluoride, poly(vinyl imidazole), chlorosulphonate polyolefins, polyethylene oxide, low density polyethylene (LDPE), high density polyethylene (HDPE) and/or blends or copolymers thereof. As such, the frame 110 can have a stiffness that can allow the frame 110 to bend, flex, elastically deform, and/or otherwise reconfigure between a first, undeformed configuration and a second, deformed configuration when exposed to an external force. More specifically, the frame 110 can have a stiffness that is sufficiently low to allow the frame 110 to transition from the first configuration to the second configuration (e.g., bend and/or deform in at least one plane when exposed to an external force) to place a surface of the therapeutic member 105 in contact with the ocular region of the user. In some embodiments, when the frame 110 is in the second configuration and when the therapeutic device 100 is coupled to a portion of the body, a force exerted by the therapeutic member 105 is distributed along the portion of the body so as to aid in a reduction of discomfort for the user, and/or to increase a surface area of the therapeutic member 105 that is in contact with the portion of the body of the user, as described in further detail herein.

The coupling portion 140 of the therapeutic device 100 is coupled to the frame 110 and can be transitioned between a first configuration and a second configuration to removably couple the therapeutic device 100 to the user. The coupling portion 140 (also referred to herein as “strap assembly”) can be any suitable shape, size, or configuration. For example, in some embodiments, the coupling portion 140 can include a first strap (not shown in FIG. 1) coupled to a first side of the frame 110 and a second strap (not shown in FIG. 1) coupled to a second side of the frame 110. The first strap can include a portion that can engage a corresponding portion of the second strap to removably couple the first strap to the second strap, thereby placing the coupling portion 140 in its second configuration. For example, in some embodiments, the first strap and the second strap can define a hook-and-loop coupling (e.g., Velcro®), a press fit, a friction fit, a snap fit, a clamp fit, and/or the like to place the coupling portion 140 in its second configuration. In other embodiments, the coupling portion 140 can include a single elastic strap that can be transitioned from a first, substantially undeformed configuration to a second, substantially deformed configuration (e.g., stretched) to place the coupling portion 140 in its second configuration.

In use, the therapeutic device 100 can be placed in a desired position relative to the ocular region of the user and the coupling portion 140 can be transitioned from its first configuration to its second configuration to substantially maintain the therapeutic device 100 in the desired position relative to the ocular region. With the coupling portion 140 in the second configuration, an area that is circumscribed by the therapeutic device 100 can be increased or decreased by decreasing or increasing, respectively, an overlapping region of the coupling portion 140. Thus, the coupling portion 140 can couple the therapeutic device 100 to users with different sized anatomy (e.g., heads).

In some embodiments, at least a part of the coupling portion 140 can be formed from a relatively flexible material (e.g., an elastic material) that can be stretched, for example, from an undeformed configuration (e.g., having an initial length) to a deformed configuration (e.g., having a relatively longer length). Thus, in some instances, a user can increase an overlapping region of the coupling portion 140 which can, for example, increase a tension within the coupling portion 140 that can be operable in coupling the therapeutic device 100 to the patient, as described in further detail herein.

In some embodiments, the coupling portion 140 can be selectively placed in the second configuration such that a desired amount of force is exerted by the therapeutic device 100 on the head of the user. For example, as described above, the coupling portion 140 can be placed in the second configuration to circumscribe a given area that can substantially correspond to a perimeter of the head of the user. Therefore, when the therapeutic member 105 is placed in contact with the ocular region of the user and the coupling portion 140 is adjusted (e.g., adjusted to increase the tension in the coupling portion 140, for example, by stretching at least a part of the coupling portion 140 such that an overlapping area thereof is increased, as described above), a force exerted by the therapeutic device 100 on the head of the user is increased. Conversely, the coupling portion 140 can be adjusted to decrease the tension in the coupling portion 140, for example, by decreasing an overlapping area of the coupling portion 140 such that a force exerted by the therapeutic device 100 on the head of the user is decreased. Thus, the coupling portion 140 can be placed in its second configuration such that a desired amount of force (as determined by the user) is exerted by the therapeutic member 105 on the ocular region of the user. Moreover, the force exerted by the therapeutic device 100 on the head of the user can be sufficient to place the frame 110 in its second configuration (e.g., can deform the frame 110). As such, a force that is, in turn, exerted by the therapeutic member 105 is distributed on the ocular region as to increase the comfort of the user and/or to increase the surface area of the therapeutic member 105 that is in contact with the ocular region, than would otherwise be possible.

The insulating portion 160 of the therapeutic device 100 can be any suitable shape, size, or configuration and can be formed from any suitable insulating material or combination thereof. For example, in some embodiments, the insulating portion 160 can be formed from a relatively thin neoprene-foam fabric material. More specifically, the insulating portion 160 can include a neoprene-foam core that is surrounded by a fabric such as, for example, spandex (e.g., Lycra®), or the like. In other embodiments, the insulating portion 160 can be formed from, for example, polyester, polyethylene terephthalate, polyester-olefin, polyester microfibers, and/or the like. In other embodiments, the insulating portion 160 can be formed from a fabric, such as a fabric derived from natural materials including felts, wools, heavy-gauge cotton, nylon fabrics, foams, plastics, woven and nonwoven materials, and/or the like.

As described above, the insulating portion 160 (also referred to herein as “insulation portion,” “insulation member,” and/or “insulating member”) can be coupled to the frame 110. More specifically, the insulating portion 160 can be transitioned from a first configuration to a second configuration to be removably coupled to the frame 110. In some embodiments, the insulating portion 160 can be substantially flat (e.g., substantially planar) when in its first configuration and can be transitioned to its second configuration in which the insulating portion 160 includes and/or defines one or more convex portions. In some embodiments, the insulating portion 160 can define a deformable portion that can be moved relative to a surface of the insulating portion 160 to place the insulating portion 160 in the second configuration. For example, in some embodiments, the insulating portion 160 can define a cut that can allow a first portion disposed on a first side of the cut to be folded onto a second portion disposed on a second side of the cut and substantially opposite the first side. In this manner, the insulating portion 160 can be transitioned from its first configuration to its second configuration in which the deforming of the insulating portion 160 results in a tenting having a convex and/or conical shape. In some embodiments, the insulating portion 160 can be placed in its second configuration to form two convex portions that substantially correspond to the first lobe and the second lobe (described above) of the frame 110. In some embodiments, the convex portions of the insulating portion 160 can substantially correspond to the portions of the therapeutic member 105 that extend through the apertures defined by the frame 110, as described above.

The insulating portion 160 can be coupled to the frame 110 via any suitable coupling. For example, in some embodiments, the insulating portion 160 can define a set of apertures that can each receive a protrusion or post that extends from an anterior surface of the frame 110. Furthermore, the arrangement of the insulating portion 160 can be such that when in the second configuration a first aperture on a first side of the cut (described above) and a second aperture on a second side of the cut are substantially aligned. Thus, the first aperture and the second aperture can be configured to receive the same protrusion or post that extends from the anterior surface of the frame 110. In some embodiments, the protrusions can include an end portion that can be flared or flanged such that when disposed in an aperture defined by the insulating portion 160, the protrusions can maintain the insulating portion 160 in the second configuration to couple the insulating portion 160 to the frame 110.

With the insulating portion 160 coupled to the frame 110, the insulating portion 160 can substantially reduce the transfer of thermal energy from an anterior surface of the therapeutic member 105 in an anterior direction. For example, the convex portions formed by the insulating portion 160 can be configured to surround at least a portion of the anterior surface of the therapeutic member 105. In this manner, surrounding at least the portion of the anterior surface of the therapeutic member 105 reduces thermal energy transfer that would otherwise be transferred from or to the anterior surface of the therapeutic member 105 (e.g., due to convection heat transfer or the like).

While the insulating portion 160 is described as being disposed in an anterior position relative to the frame 110, in some embodiments, the insulating portion 160 can include a second insulating member (not shown in FIG. 1) that can be disposed in a posterior position relative to the therapeutic member 105. For example, in some embodiments, the second insulating member can be a relatively thin sheet or fabric that can be placed onto or coupled to a posterior or anterior surface of the frame 110 and/or a posterior surface of the therapeutic member 105. In some embodiments, the second insulating member (also referred to herein as “sheet”) can include a portion that includes, for example, an adhesive or the like that can couple the second insulating member to the frame 110 and/or the therapeutic member 105. In some instances, the second insulating member can have a moisture content that can enhance the transfer of thermal energy between the ocular region of the user and the therapeutic member 105. In some instances, the second insulating member can enhance the comfort of the user while using the therapeutic device 100. For example, in some embodiments, the second insulating member can be disposed relative to the therapeutic member 105 to cover an edge of thereof and/or to cover a coupling member configured to couple the therapeutic member 105 to the frame 110 (or vice versa), that could otherwise contact the user.

In use, the therapeutic member 105 can be coupled to the frame 110 and can be configured to, for example, receive thermal energy. For example, in some embodiments, the therapeutic device 100 can be placed in a microwave oven, disposed in hot water, positioned in a heating device, electrically connected to an electrical source, and/or the like such that the potential thermal energy of the therapeutic member 105 is increased. In some instances, the insulating portion 160 can be coupled to the frame 110 or to the therapeutic member 105 after the thermal energy is transferred to the therapeutic member 105. In other instances, the insulating portion 160 can be coupled to the frame 110 or to the therapeutic member 105 prior to thermal energy being transferred to the therapeutic member 105. With the thermal energy transferred to the therapeutic member, the user can move the therapeutic device 100 to place the therapeutic member 105 in thermal conductive contact with, for example, the ocular region. Once in the desired position, the coupling portion 140 can be transitioned from its first configuration to its second configuration to couple the therapeutic device 100 to the head of the user. Thus, thermal energy can be transferred from the therapeutic member 105 to the ocular region. Moreover, the arrangement of the therapeutic device 100 can be such that the transfer of the thermal energy is substantially uniform on a desired target area of the ocular region. In addition, the force exerted by the therapeutic member 105 on the ocular region can be distributed and/or diffused in such a manner as to increase the comfort of the user while using the therapeutic device 100.

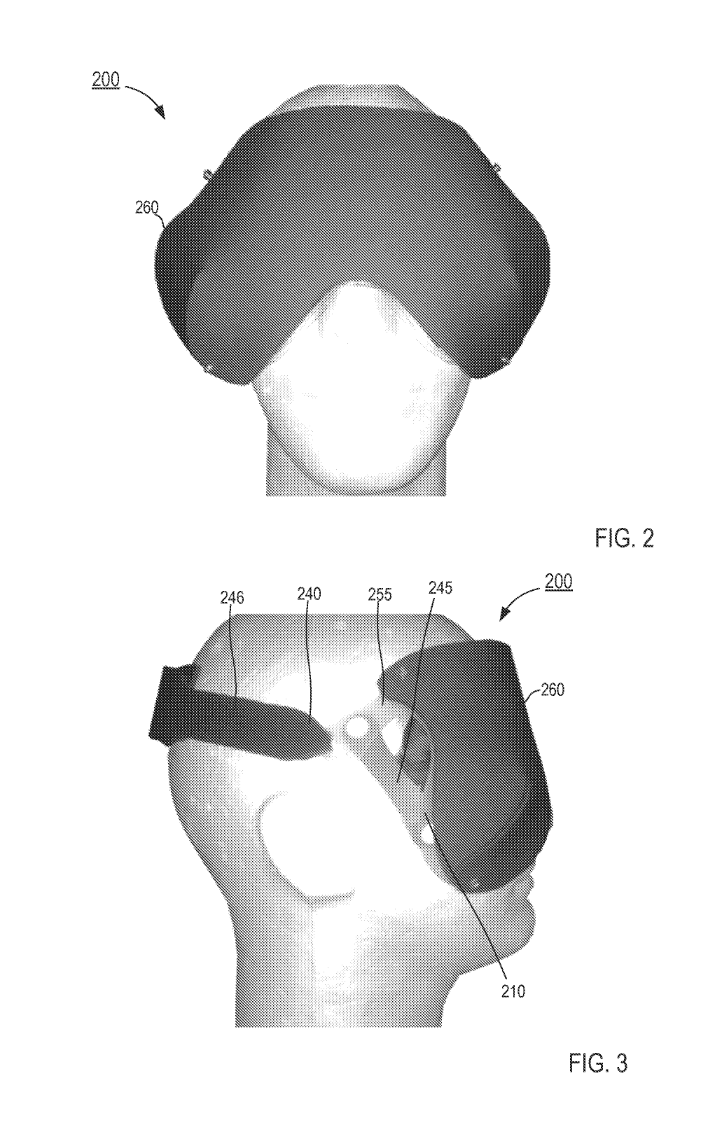

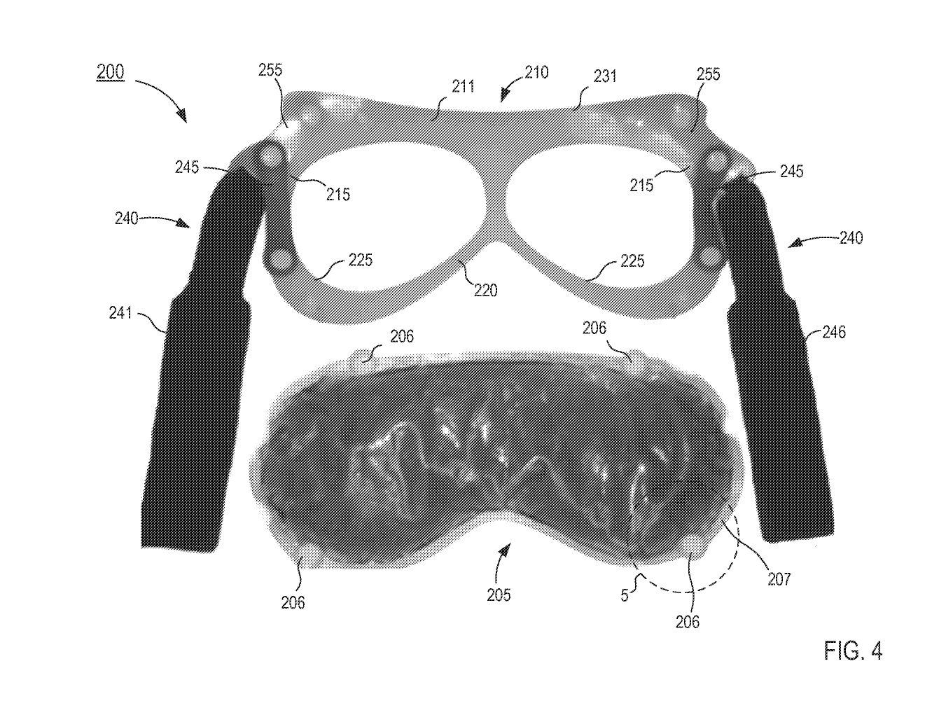

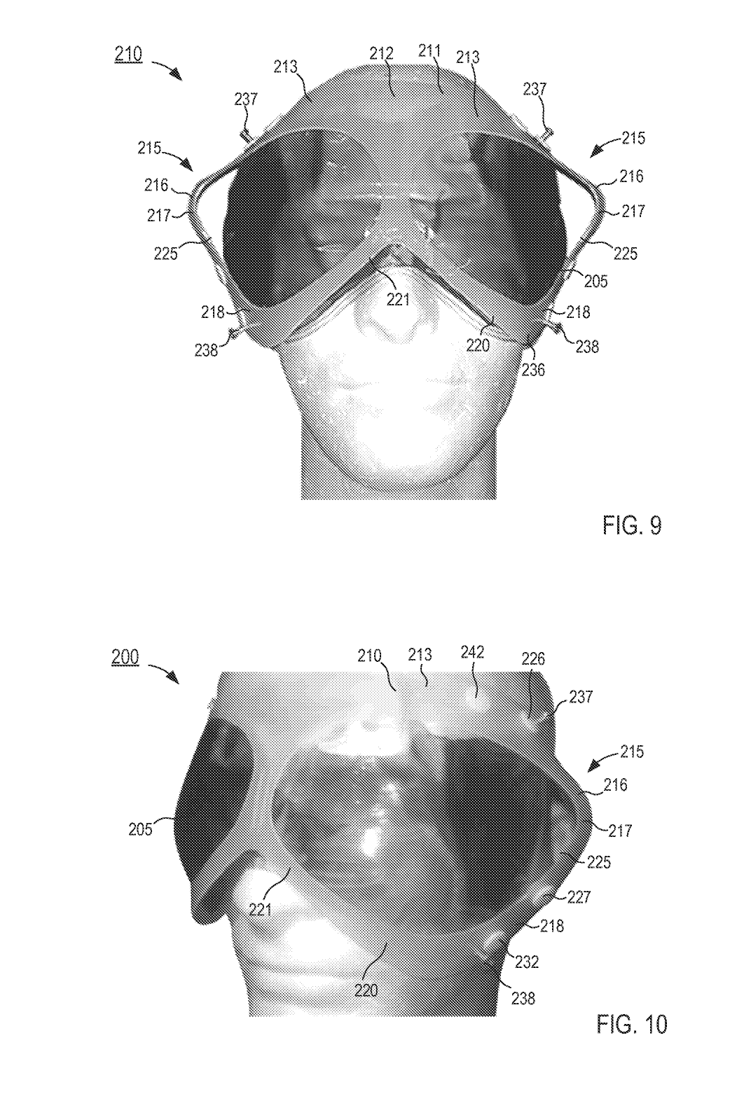

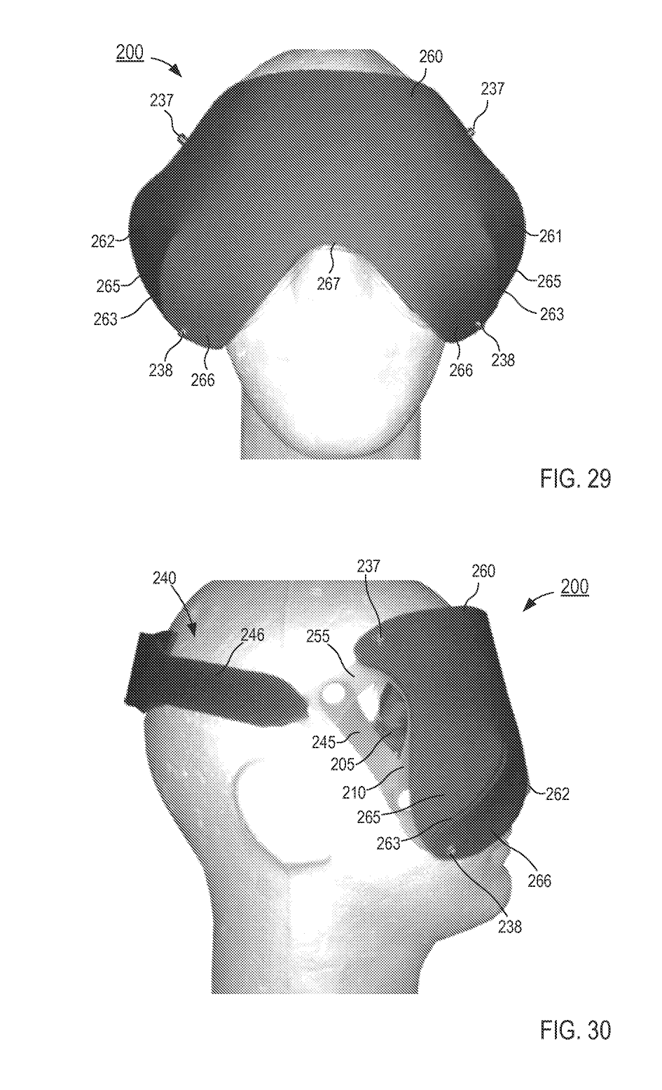

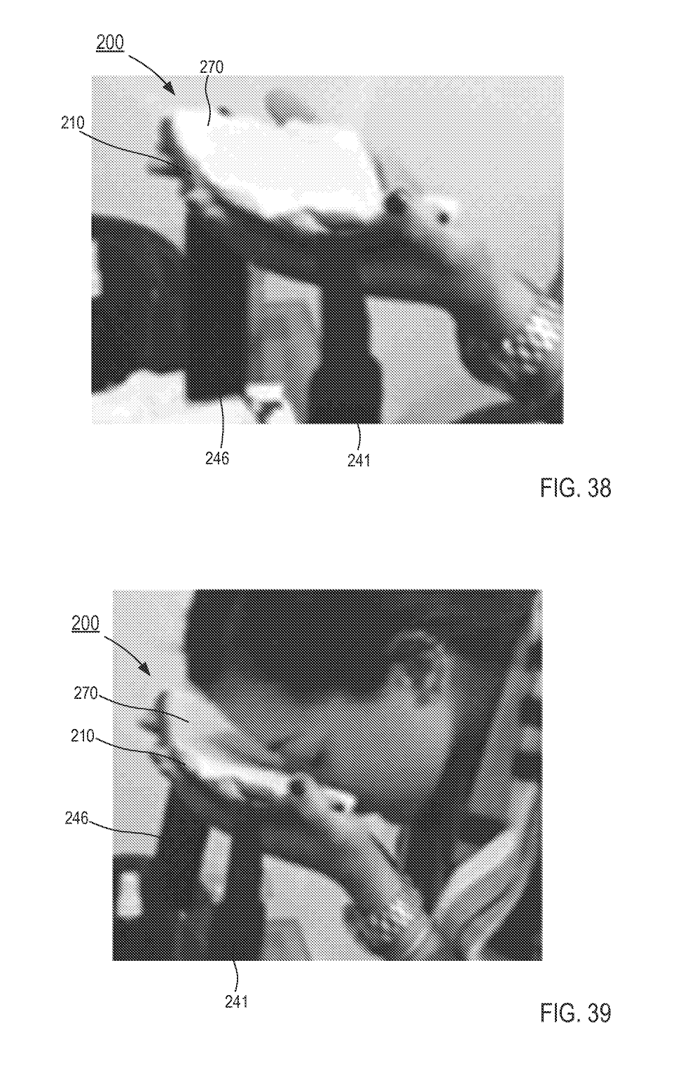

Referring now to FIGS. 2-40, a therapeutic device 200 is illustrated according to another embodiment. As shown in FIGS. 2 and 3, the therapeutic device 200 can be coupled to, for example, the head of a user to place a therapeutic member in thermal conductive contact with a target region of the face such as, for example, the ocular region. The therapeutic device 200 includes a flexible frame 210 (see e.g., FIGS. 4-10), a coupling portion 240 (see e.g., FIGS. 9-19), a first insulating member 260 (see e.g., FIGS. 20-28), and a second insulating member 270 (see e.g., FIGS. 29-33). The therapeutic device 200 (also referred to herein as “eye compress device” or “eye compress system”) can be any suitable configuration. For example, in some embodiments, the therapeutic device 200 can be an eye compress or the like such as those described in U.S. patent application Ser. No. 12/153,321 entitled, “Thermal Bodily Compress Kits and Methods of Using Same,” filed May 16, 2008; U.S. patent application Ser. No. 12/153,322 entitled, “Thermal Compress Assembly and System with External Frame,” filed May 16, 2008; U.S. patent application Ser. No. 12/947,189 entitled, “Thermal Compress System and Methods of Using the Same,” filed Nov. 16, 2010; U.S. patent application Ser. No. 13/298,445 entitled, “Thermal Bodily Compress Kits and Methods of Using Same,” filed Nov. 17, 2011; U.S. Provisional Patent Application Ser. No. 61/852,263 entitled, “Eye Compress Cover and Method of Use,” filed Mar. 15, 2013; U.S. Patent Application Ser. No. 61/962,067 entitled, “Moistened Disposable Folded Sheets for Use on an Eye Compress, and Methods of Using Same,” filed Oct. 30, 2013, and U.S. patent application Ser. No. 14/179,290 entitled, “Thermal Eye Compress Systems and Methods of Use,” filed Feb. 12, 2014, the disclosures of which are incorporated herein by reference in their entireties.

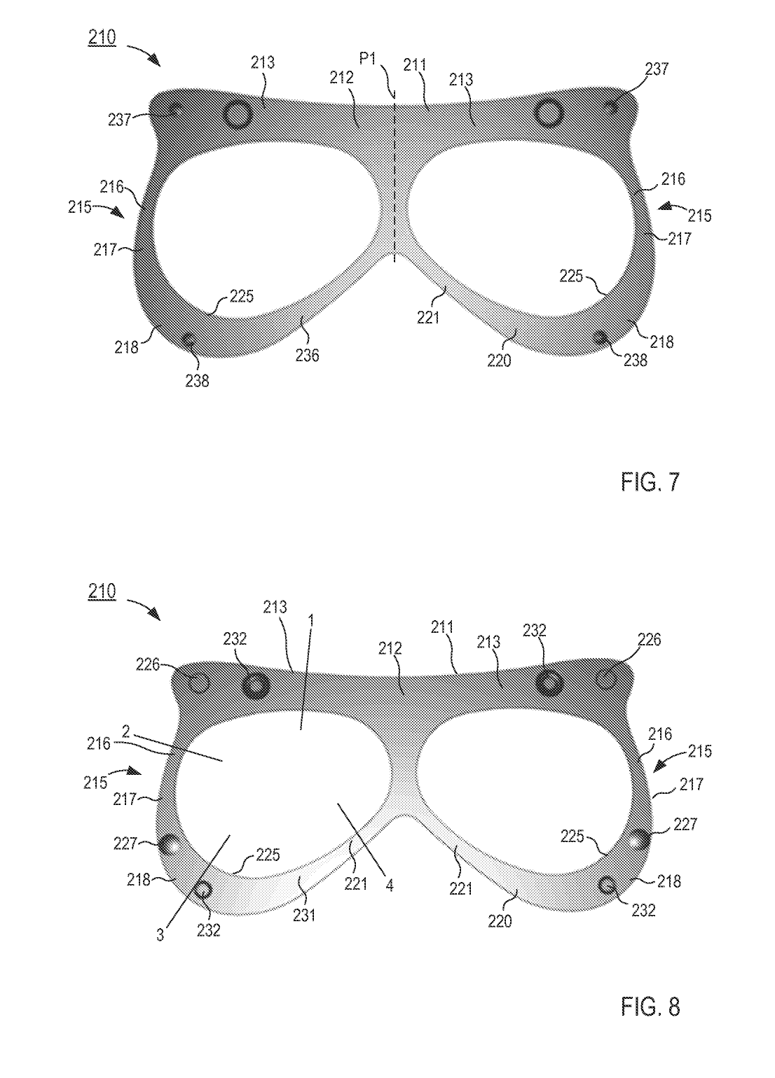

As shown in FIGS. 7 and 8, the flexible frame 210 is configured to support and/or otherwise be coupled to a therapeutic member 205. The therapeutic member 205 (also referred to herein as “thermally-adjustable therapeutic member”) can be any suitable configuration. For example, in various embodiments, the therapeutic member 205 can be substantially similar to or the same as the therapeutic member 105 described above with reference to FIG. 1. As such, the discussion of the therapeutic member 105 and/or portions thereof can apply to the therapeutic member 205 unless explicitly stated otherwise. Thus, certain aspects of the therapeutic member 205 are not described in further detail herein.

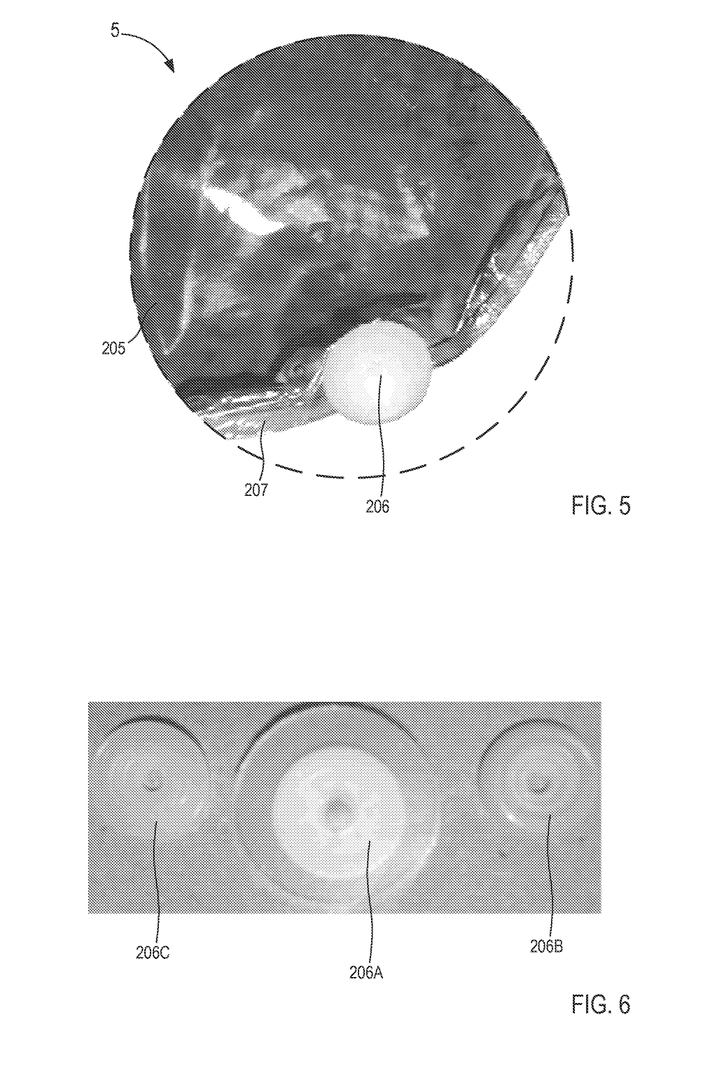

As described above, the therapeutic member 205 can include and/or can otherwise be disposed in a flexible pouch or the like. The flexible pouch can be formed from any suitable flexible material such as, for example, film material, including film material containing nylon. As shown in FIGS. 4 and 5, the flexible pouch can include a peripheral seal 207 that circumscribes the therapeutic member 205. For example, in some embodiments, at least a portion of the peripheral seal 207 can be substantially unsealed during a manufacturing process to allow, for example, a thermal gel to be disposed in an inner volume of the flexible pouch. Once a desired volume of the thermal gel or the like has been disposed in the inner volume, the peripheral seal 207 can be sealed, for example, by a second manufacturing process (or a second portion of the same manufacturing process). For example, in some embodiments, the peripheral seal 207 can be sealed to fluidically isolate the inner volume of the flexible pouch via, for example, ultrasonic welding, an adhesive, a friction weld, and/or the like.

The arrangement of the therapeutic member 205 can be such that a size and/or diameter of the peripheral seal 207 is minimized while maintaining the integrity of the seal. For example, the peripheral seal 207 can have a width (e.g., associated with a distance between, for example, an inner edge portion of the peripheral seal 207 defining a portion of the inner volume of the flexible pouch, and an outer edge portion of the peripheral seal 207 substantially opposite the inner edge portion). In some embodiments, the width of the peripheral seal 207 can be about 5 millimeters (mm). In other embodiments, the width of the peripheral seal 207 can be about 0.75 mm, about 0.9 mm, about 1.1 mm, about 1.3 mm, about 1.6 mm, about 1.9 mm, about 2.3 mm, about 2.8 mm, about 3.4 mm, about 4.2 mm, about 5.0 mm, or any fraction therebetween. In a particular embodiment, the width of the peripheral seal 207 can be about 2.5 mm. Moreover, the peripheral seal 207 can have a width, height, and/or thickness that is, for example, smaller than an overall thickness (e.g., as measured from a posterior surface to an opposite anterior surface) of the therapeutic member 205. The overall thickness can be measured, for example, when the therapeutic member 205 is resting horizontally on a flat surface and its contents have been spread evenly so that the thickness of therapeutic member 205 is substantially consistent in at least the central regions of therapeutic member 205. For example, in some embodiments, the peripheral edge 207 can have a size (e.g., a width, height, thickness, diameter, etc.) that is about 90% of the thickness of the therapeutic member 205, about 80% of the thickness of the therapeutic member 205, about 70% of the thickness of the therapeutic member 205, about 60% of the thickness of the therapeutic member 205, about 50% of the thickness of the therapeutic member 205, about 40% of the thickness of the therapeutic member 205, about 30% of the thickness of the therapeutic member 205, or less and/or any fraction of a percentage therebetween, while maintaining the integrity of the seal. As such, when the therapeutic member 205 is placed in contact with the face of a user, the peripheral seal 207 can be spaced about from the user's face (i.e., the peripheral seal 207 is not in contact with the user's face.

As shown, for example, in FIG. 4, the peripheral seal 207 can include one or more enlarged portions configured to be coupled to a coupler 206. As described above, the therapeutic member 205 can be coupled to the flexible frame 210 of the therapeutic device 200. More particularly, the therapeutic member 205 includes a set of couplers 206 that can be matingly coupled to a portion of the flexible frame 210. The couplers 206 can be any suitable shape, size, or configuration. For example, in some embodiments, each coupler 206 can be a snap or the like configured to form a snap fit with a corresponding portion of the flexible frame 210. As shown in FIGS. 5 and 6, the coupler 206 can include at least a portion that has a greater size than the remaining portions. For example, as shown in FIG. 5, the couplers 206 can be formed from a first piece 206A and a second piece 206B or 206C.

In some embodiments in which the coupler 206 is a snap or the like, the coupler can include a base 206A (also referred to herein as “first piece 206A”) and a female fastener 206B (also referred to herein as “second piece 206B”) or a male fastener 206C (also referred to herein as “second piece 206C”). In such embodiments, the base 206A includes a protrusion that extends from a cap surface of the like. The female fastener 206B and the male fastener 206C can each define an opening through which the protrusion can be inserted to fixedly couple either the female fastener 206B or the male fastener 206C thereto (as described in further detail herein). Moreover, although not shown in FIGS. 4-6, the enlarged portion of the peripheral seal 207 can include a surface that can define a sealed opening therethrough. That is to say, the enlarged portion of the peripheral seal 207 can include a surface that can define the opening such that the seal fluidically isolating the inner volume of the flexible pouch is maintained. In this manner, the protrusion extending from the base 206A of the couplers 206 can be inserted into a corresponding opening defined by a surface of the peripheral seal 207. Once inserted therethrough, either the female fastener 206B or the male fastener 206C can be disposed about the protrusion of the base 206A (e.g., on a side of the peripheral seal 207 opposite, for example, a cap portion of the base 206A) and can be fixedly coupled thereto (e.g., via a snap press, and/or any other suitable manufacturing process). Thus, the enlarged portion of the peripheral seal 207 can be compressed between the base 206A and the female fastener 206B or male fastener 206C.

In some embodiments, the enlarged portions of the peripheral seal 207 can have a size and/or shape that is associated with, for example, the base 206A and/or the female fastener 206B or male fastener 206C. For example, the peripheral seal 207 can have a size and/or shape that is sufficiently large to minimize and/or substantially eliminate the risk of tearing when the couplers 206 are being fastened to (e.g., via the snap fit or the like) or unfastened from the corresponding portions of the flexible frame 210 (i.e., during normal usage). Moreover, as shown in FIG. 6, the arrangement of the base 206A can be such that a diameter of the base 206A is significantly larger than, for example, the female fastener 206B and the male fastener 206C. Expanding further, in some embodiments, the base 206A can include a cap (from which the protrusion extends as described above) that can have an enlarged diameter relative to a cap of, for example, a comparable standard snap. For example, in some embodiments, the base 206A can have a cap diameter (e.g., an outer diameter) of about 11.0 mm, about 12.0 mm, about 13.0 mm, about 14.0 mm, about 15.0 mm, about 16.0 mm, about 17.0 mm, about 18.0 mm, or any suitable fraction therebetween. In addition, the female fastener 206B and the male fastener 206C can have an outer diameter between about 8 mm and about 10 mm. Thus, a ratio of a cap diameter or outer diameter of the coupler 206A to a cap diameter of the fastener 206B or 206A can be about 1.2, about 1.3, about 1.4, about 1.5, about 1.6, about 1.7, about 1.8, about 1.9, about 2.0, or more. In other embodiments, the arrangement of the cap diameter or base diameter of 206A can be enlarged relative to a diameter of the protrusion extending from a surface of the base 206A (described above). For example, in some embodiments a ratio of the diameter of base 206A (or cap portion of the base 206A) to the diameter of the protrusion extending from the surface of the base 206A can be about 5.25, about 5.5, about 6.0, about 6.5, about 7.0, about 7.5, about 8.0, about 8.5, about 9.0, or greater than about 9.0.

In some embodiments, the cap size of the base 206A can affect the coupling and decoupling of the therapeutic member 205 of the flexible frame 210. For example, in some embodiments, one of the couplers 206 can be coupled to a corresponding portion of the flexible frame 210 to define a snap fit. In such embodiments, the enlarged cap diameter of the base 206A can be such that a smaller force can be exerted to decouple the coupler 206 from the flexible frame 210 than would otherwise be exerted with a coupler having, for example, a standard sized cap diameter. More specifically, the arrangement of the snap fit defined between the coupler 206 and the corresponding portion of the flexible frame 210 can form and/or define a fulcrum substantially at the center of the coupler 206 (as is known in conventional snap fits). Thus, the increase in the cap diameter of the base 206A can be such that a force exerted on an edge portion of the cap in direction away from the flexible frame 210 produces a torque about the fulcrum. As such, the increase in the cap diameter increases the displacement vector of the torque, and thus, the force is reduced to generate sufficient torque to decouple the coupler 206 from the flexible frame 210. The reduction in the force can, in some instances, reduce the likelihood of tearing the enlarged portion of the peripheral seal 207 and/or can allow a user to more easily couple and decouple the therapeutic member 205 to the flexible frame 210.

Although the base 206A (i.e., the first piece) of each coupler 206 is described above as have an enlarged cap diameter, in other embodiments, a cap of the base 206A can be, for example, a standard size and a disk, washer, plate, and/or the like can be disposed adjacent to the cap of the base 206A. As such, when each of the couplers 206 is fixedly coupled to a corresponding enlarged portion of the peripheral edge 207, the disk, washer, plate, and/or the like can effectively define and/or form an enlarged cap diameter, as described above.

Referring now to FIGS. 7-12, the flexible frame 210 (also referred to herein as “frame”) of the therapeutic device 200 is coupled to the coupling portion 240 (see e.g., FIG. 4) and the first insulating member 260 (see e.g., FIGS. 2 and 3) and supports and/or is at least temporarily coupled to the therapeutic member 205. More specifically, the frame 210 has a posterior surface 231 that includes a set of couplers 232 (see e.g., FIG. 8) that can be matingly coupled to a coupler 206 (see e.g., FIGS. 4-6) included in the therapeutic member 205. The couplers 232 and 206 can be, for example, a set of snaps or the like that can be matingly engaged to removably couple the therapeutic member 205 to the frame 210, as described in detail above. Moreover, in some embodiments, the couplers 232 can each be coupled to the frame 210 in a similar manner as described above with reference to the couplers 206 and the therapeutic member 205. In other embodiments, the couplers 232 can be molded into a surface of the frame 210. In such embodiments, the molded couplers 232 can be offset a distance from the posterior surface 231 of the frame 210, which in some instances, can increase a user's comfort by decreasing a force exerted by a surface of the couplers 206 on the user's face. The frame 210 also has an anterior surface 236 that includes a set of protrusions 237 and 238 (see e.g., FIG. 7) that can engage a portion of the first insulating member 260, as described in further detail herein.

The frame 210 can be any suitable shape, size, or configuration. For example, in some embodiments, the frame 210 can be substantially similar to or the same as the frame 110 with reference to FIG. 1. As such, the discussion of the frame 210 and/or portions thereof can apply to the frame 210 unless explicitly stated otherwise. Thus, certain aspects of frame 210 are not described further herein. In some embodiments, as is shown in FIGS. 7 and 8, the frame 210 can be arranged to have a substantially smooth contour that can, for example, increase the esthetics of the frame 210. As another example, the frame 210 can be substantially symmetrical about a plane P₁ that passes through a midpoint of the frame 210, as shown in FIG. 7, such that a substantially similar or symmetric portion of the frame 210 can be placed in contact with, for example the left eye region and the right eye region.

The frame 210 can be monolithically formed from a relatively thin (e.g., between about 0.25 mm and about 4.0 mm) and flexible material such as those described above with reference to the frame 110 of FIG. 1. As such, the frame 210 can have a stiffness that can allow the frame 210 to bend, flex, elastically deform, and/or otherwise reconfigure between a first, undeformed configuration and a second, deformed configuration when exposed to an external force, as described above with reference to the frame 110. As shown in FIGS. 7 and 8, the frame 210 has a top portion 211 including a top central region 212 and superior regions 213 (relative to the user's eye); a side portion 215 including a superotemporal region 216 disposed at about a midpoint between a first attachment point 226 and a second attachment point 227 on each side portion 215, an inferotemporal region 218, and a centrolateral region 217 disposed at about a midpoint between a top edge of frame 210 and a bottom edge of frame 210; and an inferior portion 220 including an inferomedial region 221. The frame 210 also defines a set of apertures 225, as described above with reference to the frame 110. Although portions of the frame 210 are specifically described herein, certain areas, portions, and/or regions of frame 210 can overlap with other areas, portions, and regions, such that there is no clear delineation between one area, portion, or region, and its contiguously adjoining area, portion, or region. That is to say, while specific portions are described the union, intersection, and/or transition between adjacent portions need not signify a substantially change in a physical property of the frame 210.

The frame 210 can be arranged such that portions of the frame 210 have a different stiffness. For example, the arrangement of the side portion 215 can be such that the side portion 215 has a stiffness that is less than a stiffness of the top portion 211 and/or the inferior portion 220. As another example, the frame 210 can be arranged such that certain regions and/or sub-portions of the frame 210 within the side portion 215 have a stiffness that is less than a stiffness of other regions and/or sub-portions of the frame 210 within the side portion 215. The stiffness of the side portion 215 can be reduced by, for example, weakening at least the side portion 215, incorporating a different material having a lower flexural modulus into the side portion 215, incorporating a different chemical preparation in the side portion 215, forming one or more discontinuities in the side portion 215, using a different total mass of material in the side portion 215, reducing a cross-sectional area of the side portion 215, and/or the like.

By way of example, the superotemporal region 216 of the side portion 215 can have a cross-sectional area that is less than a cross-sectional area of the top portion 211, the inferior portion 220, and/or the inferotemporal region 218, as shown in FIGS. 7 and 8. More specifically, in some embodiments wherein the frame 210 has a substantially uniform thickness, the superotemporal region 216 can have a width defined between a peripheral edge of the frame 210 and an edge defining the aperture 225 that is narrower than a width (similarly defined) of the top portion 210, the inferotemporal region 218, and/or the inferior portion 220. For example, in some embodiments, the superior region 213 of the top portion 210 can have a width (taken at or about the line 1 in FIG. 8) of about 18 millimeters (mm); the superotemporal region 216 can have a width (taken at or about the line 2) of about 6.2 mm; the inferotemporal region 218 can have a width (taken at or about the line 3) of about 19.2 mm; and the inferomedial region 221 can have a width (taken at or about the like 4) of about 7.0 mm. In other embodiments, the superior region 213 of the top portion 210 can have a width between about 13.3 mm and about 18.5 mm; the superotemporal region 216 can have a width between about 6.2 mm and about 20.0 mm; the inferotemporal region 218 can have a width between about 8.5 mm and about 22.2 mm; and the inferomedial region 221 can have a width between about 6.3 mm and about 12.4 mm. In some embodiments, each portion of the frame 210 can be considered to have a cross-sectional area. Thus, for example, in a frame having a thickness of 1.27 mm, a portion of the frame having a width of 6.2 mm would have a cross-sectional area of about 7.9 mm². Hence, each portion of the frame 210 can have a specific cross-sectional area calculated in this manner, such that the relationships described above with respect to the relative widths of different portions of the frame 210 can also be applied to relative cross-sectional areas of different sections of the frame 210. Although specifically described above, the superotemporal region 216 can have any suitable width relative to another region of the frame 210. In some embodiments, the superotemporal region 216 can have a width of about 50% to about 33% of, for example, the inferotemporal region 218. In some embodiments, the superotemporal region 216 can have a width of about 90% to about 32% of, for example, the inferotemporal region 218. In other embodiments, the superotemporal region 216 can have a width of about 90%, 80%, 70%, 60%, 50%, 40%, 35%, 33%, 25%, 20%, or less of, for example, the inferotemporal region 218.

In some instances, such a reduction in width of a portion of the frame 210 from a first width to a second width can be associated with and/or can otherwise result in a reduction in a flexural modulus of that portion from a first flexural modulus to a second flexural modulus. Thus, when applied to the same force, the second flexural modulus can be expressed as a percentage of the first flexural modulus. For example, in some embodiments, the superotemporal region 216 can have a flexural modulus that is less than about 100%, 90%, 80%, 70%, 60%, 50%, 40%, 30%, 20%, or 10% of a flexural modulus of, for example, an inferotemporal region 218, when exposed to the same force. Similarly, such a reduction in width of a portion of the frame 210 from a first width to a second width can be associated with and/or can otherwise result in a reduction in a stiffness of that portion from a first stiffness to a second stiffness. Thus, the second stiffness can be expressed as a percentage of the first stiffness. For example, in some embodiments, the superotemporal region 216 can have a stiffness that is less than about 100%, 90%, 80%, 70%, 60%, 50%, 40%, 30%, 20%, or 10% of a stiffness of, for example, the inferotemporal region 218.

In this manner, at least the superotemporal region 216 of the side portion 215 of the frame 210 can be configured to form a convex bend in a portion of the frame 210 when the frame 210 is applied to a user's face, as shown in FIGS. 9-11. Similarly stated, the side portion 215 can be configured such that by reducing its stiffness, less force is required to displace the centrolateral region 217 in an anterior direction away from the user when the frame 210 is applied to the face of the user, compared to embodiments in which the side portion 215 does not have a reduced stiffness. Thus, compared to embodiments of frame 210 in which the side portion 215 does not have a reduced stiffness, the reduced stiffness of the side portion 215 can, when the therapeutic device 200 is applied to a user's face, more easily bend and/or elastically deform between the first attachment point 226 and the second attachment point 227 such that an anterior surface of the side portion 215 forms an obtuse angle (i.e., greater than 90° but less than 180°) or an acute angle (i.e., less than 90°) between the first attachment point 226 and the second attachment point 227. Thus, in some embodiments, the arrangement of the frame 210 can be such that an angle of a convex bend, measure along the posterior surface 231 of the frame 210, is decreased when the frame 210 is applied to a user's face, compared to other embodiments. For example, an angle of a convex bend can be less than 170°, less than 160°, less than 150°, less than 140°, less than 130°, less than 120°, less than 110°, or less than 100°. Increasing the degree of bending of a convex bend (e.g., by reducing the angle of the convex bend) can, in some embodiments, reduce a rearward pressure upon a portion of the therapeutic member 205 that, in turn, reduces a rearward pressure applied to the globe of the eye and/or the eye region. As such, a pressure that is transmitted to the surface of a user's closed eyelids can be reduced, which in some instances, can result in increased comfort for the user.

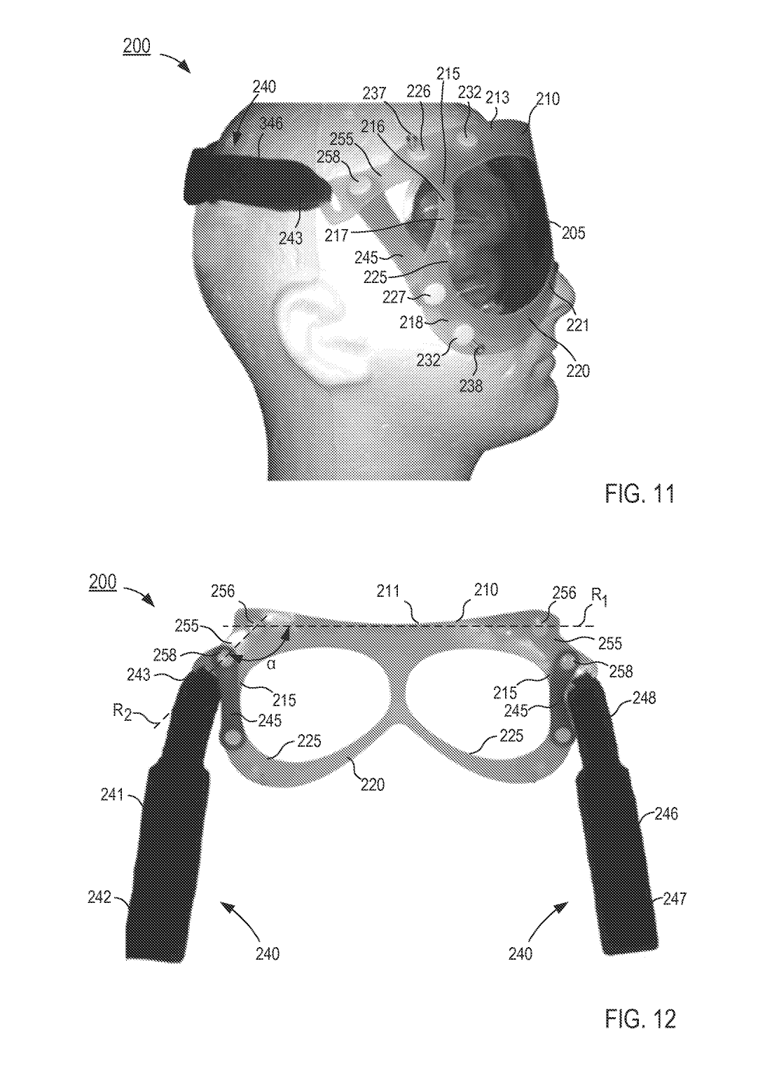

The coupling portion 240 of the therapeutic device 200 can be any suitable shape, size, or configuration. For example, in some embodiments, the coupling device 240 can be substantially similar to or the same as the coupling device 140 described above with reference to FIG. 1. As such, the discussion of the coupling portion 140 and/or components or aspects thereof can apply to the coupling portion 240 unless explicitly stated otherwise. Thus, certain aspects of the coupling portion 240 are not described in further detail herein. As shown in FIGS. 11 and 12, the coupling portion 240 (also referred to herein as “strap assembly”) can include a first strap 241 and a second strap 246 that are each coupled (either directly or indirectly) to a coupling member 255 and an inferior member 245, which in turn, can be coupled to the first attachment point 226 and the second attachment portion 227, respectively, of the frame 210. More specifically, the coupling members 255 can be formed from a substantially inelastic material. In addition, the inferior members 245 can be formed from a substantially elastic material. Thus, the straps 241 and 246 can be coupled to the corresponding coupling member 255 and the inferior member 245, thereby operably coupling the first strap 241 and the second strap 246 to the frame 210 with, for example, a desired set of characteristics, as described in further detail herein. Moreover, the coupling portion 240 can be transitioned between a first configuration (see e.g., FIG. 12) and a second configuration (see e.g., FIG. 11) to temporarily couple the therapeutic device 200 to the head of the user. As shown in FIG. 12, the first strap 241 includes a first coupling portion 242 configured to engage the second strap 246 and a second coupling portion 243 configured to couple the first strap 241 to the one of the coupling members 255 and the frame 210. Similarly, the second strap 246 includes a first coupling portion 247 configured to engage the first strap 241 and a second coupling portion 248 configured to couple the second strap 246 to the other coupling member 255 and the frame 210.

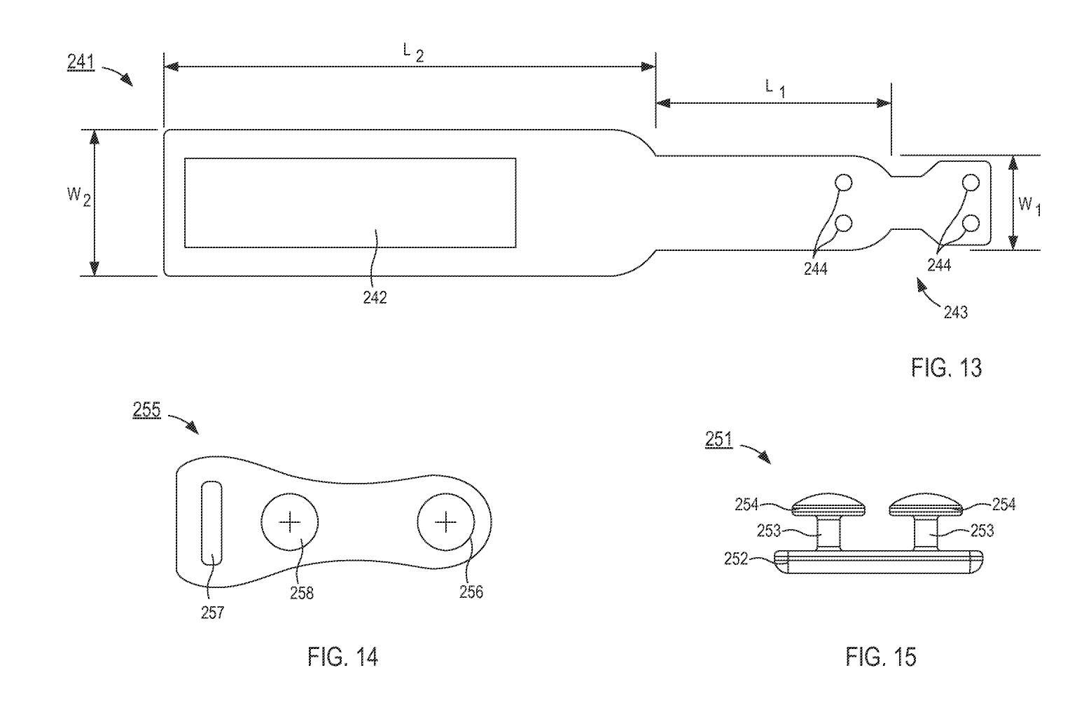

The first strap 241 and the second strap 246 can include and/or can be made of any suitable material. For example, the straps 241 and 246 can be made of a neoprene-foam-fabric material. In some embodiments, the straps 241 and 246 can have a thickness of around 1.5 mm, around 1.75 mm, around 2.0 mm, around 2.25 mm, around 2.5 mm, around 2.75 mm, around 3.0 mm, around 3.25 mm, around 3.5 mm, or any fraction therebetween. In some embodiments, the straps 241 and 246 can have a thickness less than around 1.75 mm or greater than around 3.5 mm. In some embodiments, the straps 241 and 246 formed from and/or including a neoprene-foam-fabric material and/or any other suitable material having a sufficient thickness, can improve a user's ability to grasp, hold, and use coupling portion 240. As shown in FIG. 13, the first strap 241 can have an overall shape that includes a first portion having a first width W₁ and a second portion having a second width W₂. For example, in some embodiments, the first width W₁ can be about 2.5 cm and the second width W₂ can be about 3.75 cm. In some embodiments, the width W₂ can be based at least in part on the width W₁. For example, the width W₂ can be 10%, 20%, 30%, 40%, 50%, 60%, 70%, 80%, or 90% greater than the first width W₁. In other embodiments, the width W₂ can be more than 90% greater than the width W₁. The first strap 241 can include a relatively abrupt transition from the first width W₁ to the second width W₂ that can increase the ergonomics of the first strap 241. In other embodiments, the transition from the first width W₁ to the second width W₂ can extend over a distance corresponding to a length of the strap 241, wherein the distance is configured as a percentage of the width W₁ of the strap 241. For example, in some embodiments, the transition can extend over a distance equal to about 5%, 10%, 20%, 30%, 40%, 50%, 60%, 70%, 80%, 90%, 100%, 125%, 150%, 175%, 200%, 250%, or any fraction therebetween of the width W₁ of the strap 241. In other embodiments, the transition can extend over a distance equal to about 2.5 mm, 3.75 mm, 5 mm, 6.25 mm, 7.5 mm, 8.75 mm, 10 mm, 15 mm, 20 mm, 25 mm, 30 mm, 37.5 mm, 45 mm, 50 mm, 57.5 mm, 65 mm, or any fraction therebetween.

In some instances, the arrangement of the transition from the first width W₁ to the second width W₂ can reduce the likelihood of the first strap 241 slipping through the hand of a user while placing the therapeutic device 200 against the face by providing a tactile indication to the user that the first strap 241 could be slipping. Accordingly, the user can grip the first strap 241 more tightly, which can reduce slippage. The transition from the first width W₁ to the second width W₂ can be positioned along a length of the first strap 241 that can be associated with a width of the user's hand. For example, the transition from the first width W₁ to the second width W₂ can be positioned such that when a user places the therapeutic device 200 to his or her face, the user's thumb and forefinger are positioned at and/or near the transition. Thus, when the therapeutic device 200 is transitioned to a position in which the weight of the therapeutic device 200 is no longer supported entirely by the palm, the user can grip the first strap 241 at a position along the second width W₂ and/or at about the transition between the first width W₁ and the second width W₂. In one embodiment, the first portion having the first width W₁ can have a first length L₁ and the second portion having the second width W₂ can have a second length L₂. In some embodiments, the first length L₁ can be about 6.25 cm and the second length L₂ can be about 12.5 cm. In other embodiments, the first length L₁ and the second length L₂ can have any suitable dimensions, and the first length L₁ can be any fraction of the second length L₂. For example, the first length L₁ can be about 3.75 cm, 4.5 cm, 5.0 cm, 5.75 cm, 7.0 cm, 7.5 cm, or any other suitable length. As another example, the second length L₂ can be about 7.5 cm, 10 cm, 15 cm, 18 cm, 20 cm, or any other suitable length. Similarly, the first length L₁ can be about 30%, 40%, 50%, 60%, 70%, or any other suitable fraction of the second length L₂. As such, the first length L₁ can, for example, correspond roughly to the width of a human hand. Although not shown in FIG. 13, the second strap 246 can be arranged in a similar manner. For example, the second strap 246 can include a first portion having the first width W₁ and the first length L₁, and a second portion having the second width W₂ and the second length L₂.

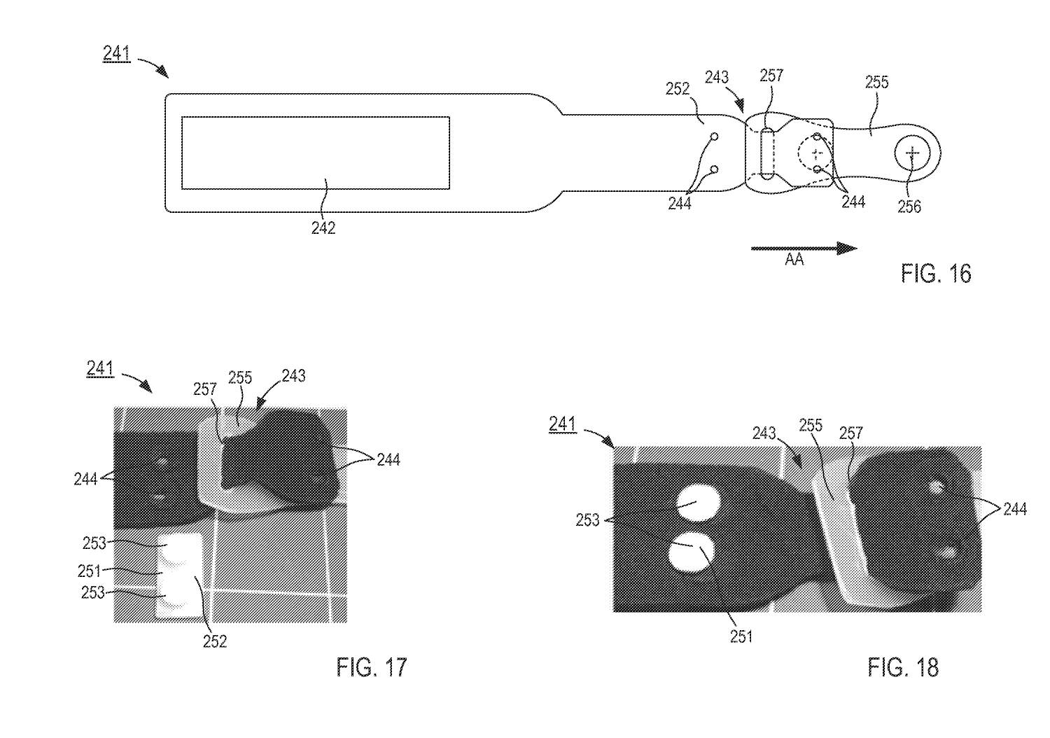

As described above, the second coupling portion 243 of the first strap 241 can couple the first strap 241 to the frame 210. The second coupling portion 248 of the second strap 246 can be the same in form and function as the second coupling portion 243 of the first strap 241. Thus, a discussion of the second coupling portion 243 of the first strap 241 also applies to the second coupling portion 248 of the second strap 246. As shown in FIGS. 13-21, the second coupling portion 243 can engage the coupling member 255 (see e.g., FIG. 14) and a closure member 251 (see e.g., FIG. 15) to be coupled to the first attachment point 226 of the frame 210. More specifically, the second coupling portion 243 can be transitioned through a set of configurations to be coupled to the coupling member 255, which in turn is coupled to the first attachment point 226 of the frame 210. Furthermore, the closure member 251 includes a base 252 and a protrusion or a set of protrusions 253 (see e.g., FIG. 16) that selectively engage the second coupling portion 243 to maintain the coupling between the first strap 241 and the coupling member 255.

In use, the second coupling portion 243 can be inserted into a slot 257 defined by the coupling member 255 to place the second coupling portion 243 in a first configuration, as indicated by the arrow AA in FIG. 16. As shown in FIGS. 17 and 18, the closure member 251 can be manipulated, for example, to place the base 252 in contact with a first side of the second coupling portion 243, and to insert, for example, the protrusions 253 through a corresponding set of openings 244 defined by the second coupling portion 243. More specifically, in some embodiments, the second coupling portion 243 can define, for example, four openings 244 that can be arranged in a rectangular orientation relative to one another, as shown in FIG. 16. Similarly stated, the set of openings 244 can be arranged in pairs of adjacent openings 244 with the pairs being aligned along a length of the second coupling portion 243 and each adjacent opening 244 in one pair being aligned along a width of the second coupling portion 243. In this manner, the protrusions 253 of the closure member 251 can be disposed in the adjacent openings 244 of one pair, as shown in FIG. 18. The protrusions 253 include a flanged end 254 (see e.g., FIG. 15) that can be pushed through the openings 244 to be disposed on a second side of the second coupling portion 243 opposite the first side, thereby placing the second coupling portion 243 in a second configuration, as shown in FIG. 18. Although the closure member 251 is shown and described above as being unitarily formed (i.e., monolithically formed and/or otherwise forming a single component), in other embodiments, a closure member can be formed of two or more units formed independently (i.e., two separate components).

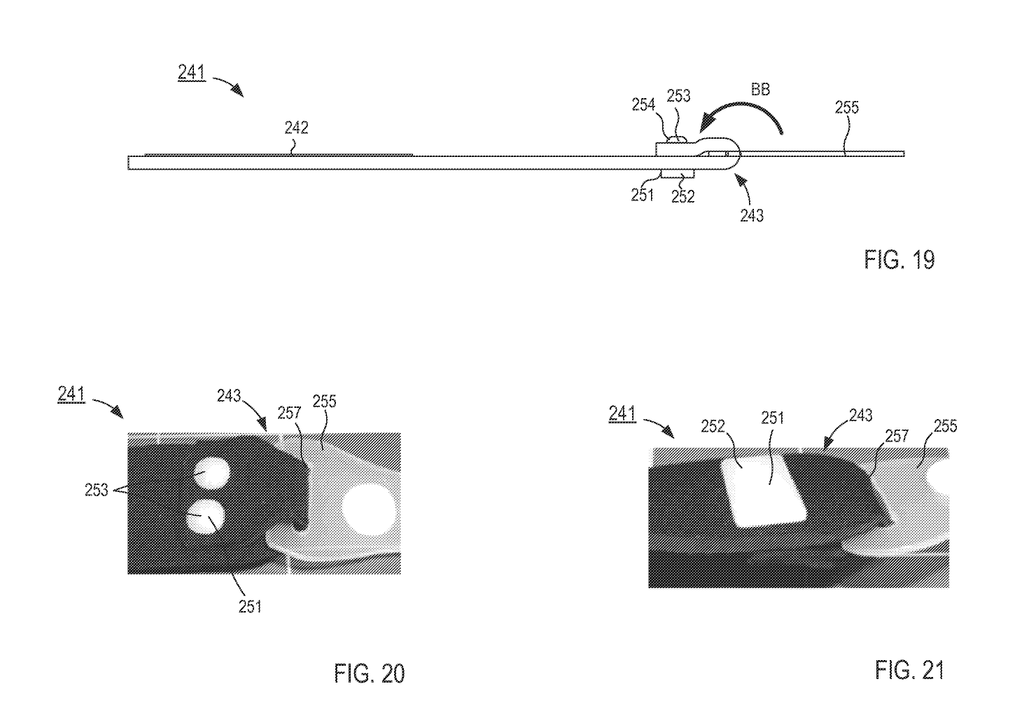

Once in the second configuration, the second coupling portion 243 can be folded about the coupling member 255 such that the two pairs of openings 244 are aligned coaxially, as indicated by the arrow BB in FIG. 19. Thus, the protrusions 253 of the closure member 251 can be inserted into the corresponding openings 244 such that the flanged end 254 extends beyond a surface of the second coupling portion 243, thereby placing the second coupling portion 243 in a third configuration. In this manner, the first strap 241 can be coupled to, for example, an outer end portion of the coupling member 255, as shown in FIGS. 20 and 21. Moreover, the coupling member 255 can include a first attachment portion 256 (e.g., disposed at and/or otherwise forming an inner end portion) that can be coupled to the first attachment point 226 of the frame 210. For example, in some embodiments, the attachment portion 256 of the coupling member 255 and the first attachment point 226 can form a snap fit or the like that can rotatably couple the coupling member 255, and therefore the first strap 241, to the frame 210. As described above, the second coupling portion 248 of the second strap 246 can be arranged in a similar manner to couple the second strap 246 to the frame 210.

Although the straps 241 and 246 are particularly shown and described above with reference to FIG. 13, in other embodiments, the straps 241 and 246 can have any suitable shape, size, and/or configuration. For example, while the strap 241 is shown in FIG. 13 as including substantially rounded corners and/or transitions between portions thereof, in other embodiments, a strap can include corners that are substantially squared. Similarly, the transitions between portions can be substantially linear. In still other embodiments, a strap can have a substantially consistent width along substantially its entire length.

While the closure member 251 is particularly shown and described above with reference to, for example, FIG. 15, in other embodiments, a closure member can have any suitable shape, size, or configuration. For example, in some embodiments, while shown as having substantially rounded corners and/or transitions, in other embodiments, a closure member can have any suitable shape such as substantially rectangular, square, and/or any other suitable polygonal shape. Moreover, while the closure member 251 is shown as including two protrusions 253 that extend from the same side of the base 252, in other embodiments, a closure member can include, for example, four protrusions with two sets of two protrusions being disposed on opposite surfaces of a base. As such, the base of the closure member can be disposed between, for example, a first portion of a strap and a second portion of that strap to place the strap in its third configuration. In other embodiments, a closure member can include less than two protrusions (i.e., a single protrusion). In still other embodiments, a closure member can be unitarily or monolithically formed with a coupling member. As such a set of protrusions can extend from at least one surface of the coupling member to be disposed in a corresponding set of openings defined by a strap, as described above.

Referring back to FIGS. 11 and 12, the inferior members 245 are coupled to a second attachment portion 258 of the coupling members 255 and are configured to extend at an angle therefrom to allow the inferior members 245 to be coupled to the second attachment points 227 of the frame 210. For example, the inferior members 245 can form a snap fit or the like with the second attachment portion 258 of the coupling members 255 and the second attachment points 227 of the frame 210. Furthermore, the arrangement of the coupling members 255 and the inferior members 245 can form a bifid or V-shape, in which the inferior members 245 extend from the second attachment portion 258 toward the frame 210 at an angle relative to the coupling member 255. Similarly stated, the second attachment points 227 of the frame 210 are disposed in an inferior position relative to the first attachment points 226 and as a result the inferior members 245 traverse a space defined between the coupling member 255 and the second attachment points 227 such that the inferior members 245 are oriented at an angle relative to the coupling members 255. In some embodiments, the coupling members 255 can have any suitable length or dimension. In other embodiments, however, the coupling members 255 can have a length such that the rear portion of coupling members 255 is positioned anterior to a pinna of a user's ear, a midpoint of a user's ear, or an anterior portion of a user's ear, when the therapeutic device 200 is applied to the user's eye region.

When the user places the coupling portion 240 in the second configuration to couple the therapeutic device 200 to his or her head, the straps 241 and 246 can exert a force on the coupling members 255 that helps to maintain the therapeutic device 200 in a desired position. The arrangement of the coupling members 255 and the inferior members 245 is such that the force exerted by the straps 241 and 246 is distributed between the coupling members 255 and the inferior members 245 which, in turn, exert a portion of the force on the first attachment point 226 and the second attachment point 227, respectively. Thus, the force exerted by the straps 241 and 246 is distributed along a length of the side portion 215 of the frame 210. Moreover, in embodiments in which at least a portion of the superotemporal region 216 and/or the centrolateral region 217 is thinned, the distributed force can elastically deform the side portions 215 of the frame 210 more easily than embodiments in which at least the portions are not thinned. In this manner, the force exerted by the frame 210 and the straps 241 and 246 to couple to the therapeutic device 200 to the user can be distributed along the user's head in a manner that can increase a user's comfort and/or can increase a surface area of the therapeutic member 205 in contact with the ocular region of the user.

Expanding further, as described above, the straps 241 and 246 exert a rearward force (for example, toward the rear of the user's head) upon frame 210, which is transmitted through therapeutic member 205 and hence, upon the eye region of the user. The bifid or V-shaped strap arrangement of the coupling portion 240 can divide and/or otherwise distribute the rearward force vector of the straps 241 and 246 into a pair of force vectors exerted on the frame 210, one directed above and one directed below the eye level of the user, thus reducing pressure directly upon the eyes of the user and in some instances, increasing user comfort. In embodiments in which the coupling members 255 are formed from a relatively inelastic material and the inferior members 245 are formed from a relatively elastic material, a greater outward or convex bending of the side portions 215 of the flexible frame 210 can result from the therapeutic device 200 being placed on a user's head, than would otherwise result in embodiments in which, for example, the coupling members 255 are formed from a relatively elastic material. Thus, for example, the force exerted by the straps 241 and 246 upon the frame 210 can displace a region associated with attachment point 226 in an posterior-inferotemporal direction, and can displace the inferotemporal region 218 associated with attachment point 227 in a posterior-superotemporal direction.

More specifically, in some embodiments, the coupling members 255 can be formed from a relatively inelastic material such as, for example, nylon, LDPE, HDPE, or other plastic having a relatively high flexural modulus, and the inferior members 245 can be formed from a relatively elastic material such as, for example, silicone, silicone rubber, and/or any other elastomer with a relatively low flexural modulus. In this manner, a force applied along a length of the coupling members 255 results in little or no elongation of the coupling members 255, while a similar force applied along a length of the inferior members 245 results in a desired amount of elongation, greater than an elongation of the coupling members 255 (e.g., elastic or nonpermanent elongation). In other embodiments, the inferior members 245 can be formed from and/or can include a material having a relatively high flexural modulus, including materials substantially similar to the materials of coupling members 255.

The arrangement of the coupling members 255 and the inferior members 245 can be such that the coupling members 255 and the inferior members 245 are disposed at a respective angle relative to the frame 210. For example, as shown in FIG. 12, the arrangement of the frame 210 can be such that a first reference axis R₁ extends through substantially the center of the first attachment points 226 (not shown in FIG. 12) and thus, the first attachment portions 256 of the coupling members 255. Similarly, a second reference axis R₂ extends through substantially the center of the first attachment portion 256 and the second attachment portion 258 of the coupling member 255, or said another way, the second reference axis R₂ substantially longitudinally bisects the coupling member 255. As such, a coupling member angle α is defined therebetween. As shown in FIG. 12, the coupling member angle α is less than, for example, 180°. In some embodiments, the coupling member angle α can be, for example, about 110°, about 120°, about 130°, about 135°, about 140°, about 150°, about 160°, about 170°, or any angle or fraction of an angle therebetween. Hence, the coupling members 255 are disposed at the coupling member angle α relative to the first reference axis R₁. Said another way, the coupling members 255 extend in the inferotemporal direction from an axis associated with the first attachment points 226, for example, at least while the therapeutic device 200 is in a first configuration (e.g., not coupled to the head of a user).

In use, such an arrangement of the coupling members 255 and the inferior members 245 can be such that a force resulting from the coupling portion 240 being transitioned to its second configuration is distributed along the side portion 215 of the frame 210 is a desired manner. For example, in some instances, a user can position the therapeutic device 200 adjacent to his or her face and can exert a force of the straps 241 and 246 to place the coupling portion 240 in the second configuration (e.g., to couple the straps 241 and 246 together). As the user exerts the force on the straps 241 and 246 in a substantially posterior direction, a tension in the straps 241 and 246, in turn, exerts a force on the coupling members 255. With the straps 241 and 246 disposed in a substantially inferior or inferotemporal position prior to the therapeutic device 200 being placed in contact with the user's face, a rearward force applied to the straps 241 and 246 can move the straps 241 and 246 to less inferior positions, thereby resulting in a superior-posterior force upon the straps 241 and 246. In turn, the straps 241 and 246 exert a force substantially in the same direction (that is, a substantially superior-posterior direction) on the coupling members 255 which can result in, for example, a torque or moment exerted at and/or substantially near the center of attachment point 226 (e.g., substantially at the intersection of the reference axes R₁ and R₂). Therefore, the inelastic configuration of the coupling member 255 is such that the coupling member 255 acts as a lever having a fulcrum at frame attachment point 226 and as a result, the coupling member angle α is increased. Moreover, with the inferior member 245 coupled to the second attachment portion 258 of the coupling member 255 the resulting torque exerts a force on the inferior member 245 in an upwards or superoposterior direction.

In embodiments, the combination of the substantially inferotemporally-angled straps 241 and the substantially inelastic coupling members 255 can function in a synergistic manner to potentiate the torque or moment of force at or about the first attachment point 226 of the frame 210 and thus, a force is exerted upon inferior member 245 in the superoposterior direction. Moreover, with the inferior members 245 coupled to the second attachment points 227, the inferior members 245, in turn, exert a force upon the second attachment points 227 of the frame 210 in the superoposterior direction. The force acting upon the second attachment points 227 can displace the inferotemporal region 218 of frame 210 in a superoposterior manner. As the inferotemporal region 218 of the frame 210 is displaced, the inferotemporal region 218 is brought into a more proximate or close alignment with a user's face. Hence, in some embodiments such as those described above, the inferotemporal region 218 exerts more force against a portion of the user's face, such as a cheek area or an upper cheek area, than in other embodiments. The location of this force can, in some embodiments, improve a desired configuration of the therapeutic member 205 in relation to the portion of the user's face, for example, by increasing an area of the therapeutic member 205 that is in thermal conductive contact with the portion of the user's face.

The displacement of the inferotemporal region 218 of the frame 210 further exerts a force that can, for example, elastically deform the side portion 215 of the frame 210. The arrangement of the frame 210 can be such that the side portions 215 elastically deform (i.e., nonpermanently deform or otherwise reconfigure) in a convex and generally anterotemporal direction in response to the forces exerted on the frame 210 by the coupling portion 240. In some embodiments, the outward or convex bending of the side portions 215, especially when such a convex bending is positioned at and/or near the eye level of the user, can, for example, result in a reduced rearward pressure upon therapeutic member 205. Thus, a rearward pressure exerted upon the user's eyes is less than a rearward pressure that would otherwise be exerted by a frame having side portions of greater stiffness (e.g., side portions that were not configured to outwardly or convexly bend, as described above). Said another way, forces exerted by the coupling portion 240 and/or the frame 210 that would otherwise be directed rearward toward the eye regions and that could, for example, result in an increase in pressure upon the eyes, and/or an increase in ocular discomfort, are converted and/or distributed by the anterotemporal bending of the frame 210, into forces that are directed anterotemporally away from the eye region, which can, for example, result in a decreased pressure upon the eyes, and/or can, for example, result in greater user ocular comfort. In addition, the convex bending of the frame 210 shortens a vertical distance between the upper couplers 232 and the lower couplers 232 of the frame 210, thereby reducing a tension within a portion of the therapeutic member 205 that can otherwise result from a stretching of therapeutic member 205 between the upper couplers 232 and the lower couplers 232 of the frame 210. Thus, as the tension is decreased, the therapeutic member 205 can assume a less rigid configuration, which in turn, can reduce the pressure placed upon the eyes, and/or can reduce the ocular discomfort of the user.

Although the frame 210 and/or coupling portion 240 are specifically described above, in other embodiments, the frame 210 and/or the coupling portion 240 can be arranged in any suitable manner that can, for example, increase the convex bending of a portion of the frame 210 to decrease a direct pressure exerted on the eyes, and/or can reduce user discomfort. For example, by weakening or thinning at least a portion of the superotemporal region 216 and/or the centrolateral region 217, a distributed force can result in an amount of elastic deformation of the side portions 215 of the frame 210 that is greater than an amount of elastic deformation of a side portion not weakened or thinned. In some instances, a reduction in pressure exerted on the eye region, and/or a reduction in user discomfort (i.e., an increase in user comfort) can, for example, allow a user to more easily apply therapeutic device 200 in a close-fitting and therapeutic manner, which in turn, can improve therapeutic outcomes. As described above, the apertures 225 defined by the frame 210 allow a portion of the therapeutic member 205 to extend anteriorly, away from the eyes and substantially without resistance, when the therapeutic device 200 is coupled to the head of the user. Thus, the arrangement of the apertures 225 can further reduce a force exerted by the therapeutic member 205 directly toward the globe of the eye. As such, the apertures 225 and hence, the reduction of posteriorly-directed forces within the region of the apertures 225, can further reduce the pressure upon the globe of the eye, and/or can increase the comfort of the user.

While the arrangement of the side portions 215 and the apertures 225 of the frame 210 reduce a rearward pressure exerted on the eyes of the user, the relatively larger surface area of the frame 210 substantially at and/or near the inferotemporal region 218 can, for example, exert a force upon a larger surface area, and in some embodiments upon a larger volume, of therapeutic member 205. In some embodiments, the larger surface area of the inferotemporal region 218 can be such that when the straps 241 and 246 exert a force on the frame 210 (as described above), at least a portion of the inferotemporal region 218 is displaced in a superoposterior direction, which in turn can displace a greater volume of contents of the therapeutic member 205 substantially in the same direction. More specifically, in some instances, such contents of the therapeutic member 205 can be displaced in the superoposterior direction up from a cheek region and toward the eye of the user. Similarly, the relatively large surface area of the superior region 213 of the frame 210 can displace a volume of content of the therapeutic member 205 in an inferoposterior direction. Thus, the force exerted by the inferotemporal region 218, and also in some embodiments by superior region 213, can be such that a volume of the contents (e.g., thermal gel) included in the therapeutic member 205 is pushed and/or directed toward the ocular region of the user. Moreover, by allowing the side portion 215 to bend in an anterolateral or convex direction (as described above), the inferotemporal region 218, and also in some embodiments by superior region 213, can direct a volume of the contents of the therapeutic member 205 in such a way that the total effect of force exerted by frame 210 upon the therapeutic member 205 is smaller than would otherwise be possible without the selective bending or flexing of the side portion 215. Moreover, with a portion of the therapeutic member 205 extending through the apertures 225 (as described above), a force exerted by the therapeutic member 205 directly toward the globe of the eye is reduced, which in some embodiments can thereby further increase comfort of the user.

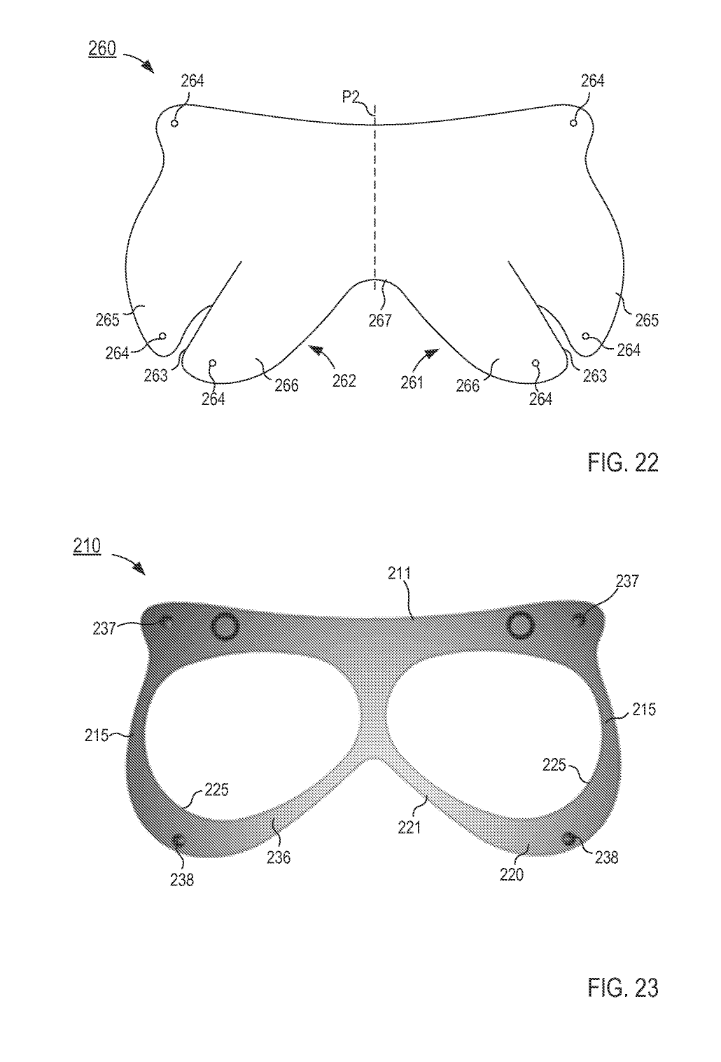

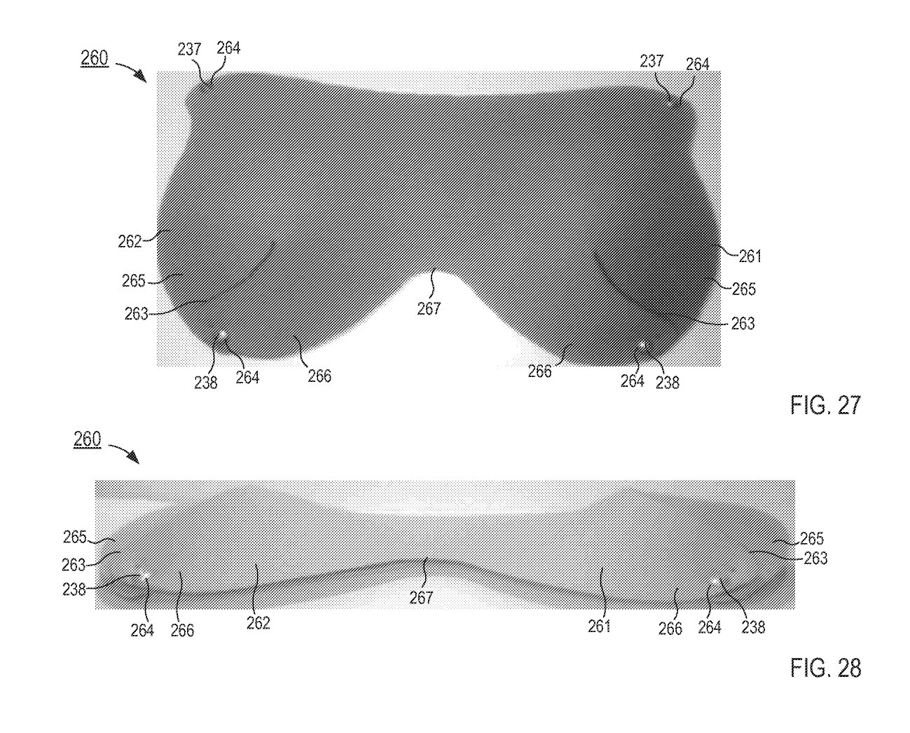

Referring now to FIGS. 22-30, the first insulating member 260 can be transitioned from a first configuration (FIGS. 22, 23, 25 and 26) to a second configuration (FIGS. 27-30) to be coupled to an anterior surface of the frame 210. The first insulating member 260 (also referred to herein as “insulating portion,” “insulation portion,” and/or “insulation member”) can be any suitable shape, size, or configuration and can be formed from any suitable insulating material or combination thereof. For example, the first insulating member 260 can have a shape that is associated with the frame 210 and can be formed from a relatively thin neoprene-foam fabric material. In some embodiments, the first insulating member 260 can be formed from a material having a thickness of less than about 0.5 mm, less than about 0.75 mm, less than about 1.0 mm, less than about 1.25 mm, less than about 1.5 mm, less than about 1.75 mm, less than about 2.0 mm, less than about 2.25 mm, or less than about 2.5 mm. In other embodiments, the first insulating member can be formed from a material having a thickness of 2.5 mm or more. In some embodiments, the first insulating member 260 can be a cover or the like such as those described in U.S. Provisional Patent Application Ser. No. 61/852,263 entitled, “Eye Compress Covers and Methods of Use,” filed Mar. 15, 2013, the disclosure of which is incorporated herein by reference in its entirety. In some embodiments, the first insulating member 260 can include a neoprene-foam core that is surrounded by a fabric such as, for example, spandex (e.g., Lycra®), or the like. In other embodiments, the first insulating member 260 can be formed from, for example, polyester, polyethylene terephthalate, polyester-olefin, polyester microfibers, and/or the like. In other embodiments, the first insulating member 260 can be formed from fabrics derived from natural materials including felts, wools, heavy-gauge cotton, nylon fabrics, foams, plastics, woven and nonwoven materials, and/or the like.

As shown in FIG. 22, the first insulating member 260 is substantially flat (e.g., substantially planar or including an anterior surface and a posterior surface that are substantially parallel and two dimensional) when in its first configuration. The first insulating member 260 has a first lobe 261 and a second lobe 262 at least partially defined and/or separated by a nasal region 267, and defines a set of openings 264. The arrangement of the first insulating member 260 can be such that the first lobe 261 and the second lobe 262 are substantially similar in size and shape while being oriented in opposite directions. Similarly stated, the first insulating member 260 is substantially symmetrical about a plane P₂ that passes through a midpoint of the nasal region 267, as shown in FIG. 22. Thus, a discussion of the first lobe 261 of the first insulating member 260 also applies to the second lobe 262 of the first insulating member 260.

The first lobe 261 defines a slit 263 that separates a portion of the first lobe 261 into a first leaflet 265 disposed on a first side of the slit 263 and a second leaflet 266 defined on a second side of the slit 263. The first lobe 261 can be arranged such that a width of the slit 263 varies along its length. As an example, the width of the slit 263 can increase along length. More particularly, the slit 263 can have a first width at an origin (e.g., a first end portion in a position that generally corresponds to the center of the eye region) and a second width at a peripheral position (e.g., a second end portion in a position that generally corresponds to a peripheral edge of the first insulating member 260). In some embodiments, the arrangement of the slit 263 can be such that as the slit 263 transitions from the first width to the second width, a wedge-shaped space is defined between the first leaflet 265 and the second leaflet 266, as shown in FIG. 22. In some embodiments, the first leaflet 265 can include a substantially curvilinear edge surface and the second leaflet 266 can include a substantially linear edge surface, which collectively define the slit 263. In some embodiments, the slit and/or an edge surface of the leaflets 265 and 266 can be configured to selectively position the leaflets 265 and 266 relative to one another when the first insulating member 260 is in the second configuration, as described further below. In some embodiments, a peripheral or distal portion of the first leaflet 265 and the second leaflet 266 that collectively define a portion of the slit 263 can be substantially rounded.

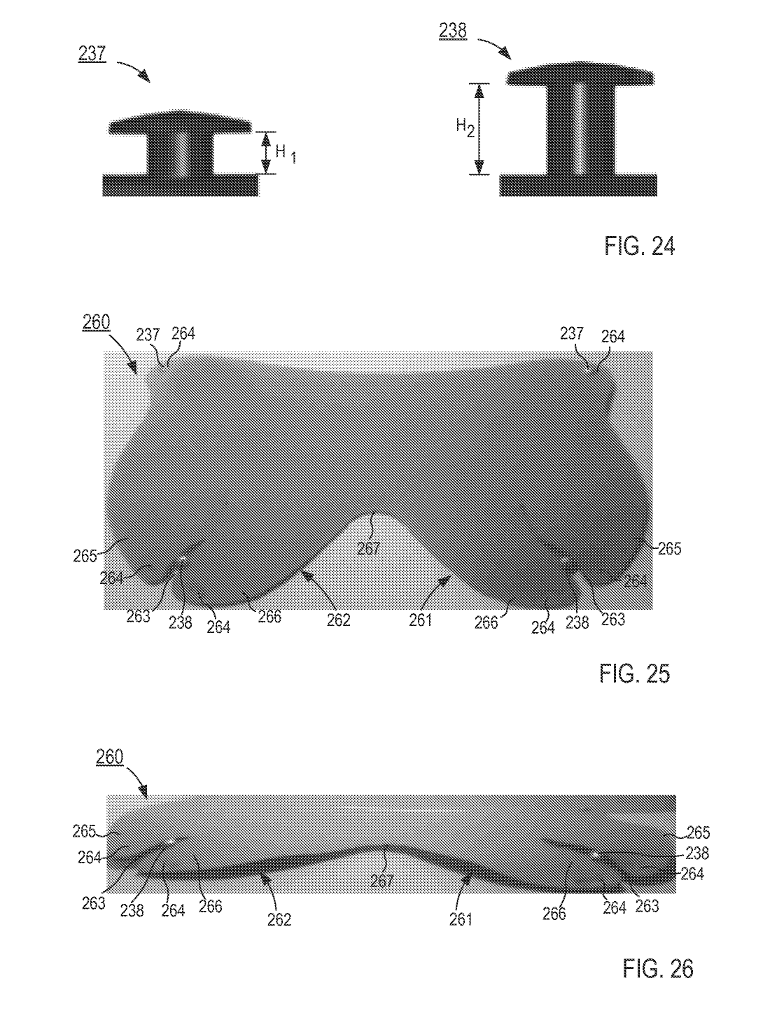

The arrangement of the slit 263 can allow the second leaflet 266 to be moved relative to the first leaflet 265 (or vice versa) to couple the first lobe 261 to the frame 210. Furthermore, portions of the second lobe 262 can be moved in a similar manner to couple the second lobe to the frame 210. For example, as shown in FIGS. 23 and 24, the anterior surface 236 of the frame 210 includes a first set of posts 237 and a second set of posts 238. The first set of posts 237 are disposed along the top portion 211 of the frame 210 and the second set of posts 238 are disposed along the inferior portion 220 of the frame 210. As such, the first set of posts 237 and the second set of posts 238 can be inserted into the set of openings 264 defined by the first insulating member 260 to couple the first insulating member 260 thereto. As shown in FIG. 24, the first set of posts 237 and the second set of posts 238 can each include a flanged end portion that can be placed in contact with an anterior surface of the first insulating member 260, when disposed in the openings 264, to at least temporarily maintain the position of the first insulating member 260 relative to the frame 210. Furthermore, the first set of posts 237 can have a first height H₁ defined between a base and the flanged end that can substantially correspond with a width of the first insulating member 260. Similarly, the second set of posts 238 can have a second height H₂ defined between a base and the flanged end that can about twice the width of the first insulating member 260, as described in further detail herein.

As described above, the first insulating member 260 can be transitioned from a first configuration to a second configuration to be coupled to the frame 210. For example, FIGS. 25 and 26 illustrate the first insulating member 260 in the first configuration and partially coupled to the frame 210 (e.g., the first set of posts 237 are disposed in the openings 264 along a top of the first insulating member 260). As shown in FIGS. 27 and 28, the first lobe 261 and the second lobe 262 can be manipulated to place the first insulating member 260 in the second configuration, thereby coupling the first insulating member 260 to the frame 210. For example, the first lobe 261 and the second lobe 262 can be manipulated to insert the second set of posts 238 in the openings 264 defined by the first leaflets 265 and the second leaflets 266 can be moved relative to the first leaflets 265 to insert the second set of posts 238 in the openings 264 defined by the second leaflets 266. As a result, a portion of the second leaflets 266 overlaps a portion of the first leaflets 265 so that the leaflets 265 and 266 substantially fully cover the front portion of therapeutic member 205 substantially without any gaps being present between leaflets 265 and 266. Moreover, the arrangement of the slit 263 can increase the flexibility of the lobes 261 and 262 as well as minimize material that would otherwise bunch, obstruct, and/or prevent the first leaflets 265 and/or the second leaflets 266 from fully overlapping one another and thereby fully covering the front portion of therapeutic member 205. Furthermore, the height H₁ of the first set of posts 237 and the height H₂ of the second set of posts 238 can be such that the flanged ends are in contact with the anterior surface of the first insulating member 260 to at least temporarily retain the position of the first insulating member 260 relative to the frame 210.

As shown in FIGS. 27-30, by moving the first leaflets 265 and the second leaflets 266 relative to one another, the first lobe 261 and the second lobe 262 are deformed. Similarly stated, when the first insulating member 260 is in its second configuration, the first lobe 261 and the second lobe 262 are transitioned from having an anterior surface that is substantially flat or two-dimensional to having the anterior surface that is not flat or three-dimensional. For example, the first lobe 261 and the second lobe 262 can form a convex and/or conical shape that extends in the anterior direction. More specifically, moving the second leaflets 266 such that the portion of the second leaflets 266 overlaps the portion of the first leaflets 265 results in a tenting of the first lobe 261 and the second lobe 262. In this manner, the first insulating member 260 can be placed in its second configuration to form two convex portions that substantially correspond to the first lobe and the second lobe (described above) of the frame 210. In some embodiments, the relative position of the openings 264 defined by the first leaflets 265 and the second leaflets 266 can modify one or more characteristics of the convex portion. For example, in some embodiments, the openings 264 can be disposed at a predetermined distance from a surface (i.e., a periphery surface or the like) of the first leaflets 265 and the second leaflets 266 that defines the slit 263. By way of example, in some embodiments, the openings 264 can be about 5 millimeters (mm) from the surface, about 10 mm from the surface, about 14 mm from the surface, about 20 mm from the surface, about 30 mm from the surface, about 50 mm from the surface about 75 mm from the surface, about 100 mm from the surface, or any distance or fraction of distance therebetween. Moreover, the relative positioning of the opening 264 defined by the first leaflets 265 can be different from a relative positioning of the opening 264 defined by the second leaflets 266. For example, the opening 264 defined by the first leaflets 265 (e.g., lateral leaflets) can be less than 10 mm from the surface, while the opening 264 defined by the second leaflets 266 (e.g., medial leaflets) can be greater than 10 mm from the surface.

In some embodiments, the convex portions of the first insulating member 260 can substantially correspond to the portions of the therapeutic member 205 that extend through the apertures defined by the frame 210, as described above. For example, the convex shape of the first lobe 261 and the second lobe 262 can define a posterior volume (not shown) that can receive the portions of the therapeutic member 205. As a result, the first insulating member 260 surrounds at least the portion of the anterior surface of the therapeutic member 205 to reduce thermal energy that would otherwise be transferred from or to the anterior surface of the therapeutic member 205 (e.g., due to convection heat transfer or the like). Moreover, the convex shape of the first lobe 261 and the second lobe 262 reduces a force that would otherwise be exerted by a substantially flat or non-convex first insulating member on the anterior surface of the therapeutic member 205, which reduction in force can increase a user's comfort level. In addition, the first insulating member 260 can include and/or be formed from a material (e.g., neoprene, neoprene foam, other foam materials, and/or the like) that can have a stiffness that is sufficient to remain in the second configuration without external support of the convex portion of the first lobe 261 and the second lobe 262.

Although the first insulating member 260 is particularly shown and described above, in other embodiments, a first insulating member can be any suitable shape, size, and/or configuration such that, when coupled to the flexible frame 210, the first insulating member provides a thermal insulation in the anterior direction. For example, although the slits 263 of the first insulating member 260 are shown in a particular orientation and/or arrangement, in other embodiments, a first insulating member can include one or more slits disposed at any suitable position along the periphery of the first insulating member (e.g., at or along a side portion). Similarly, while the slits 263 are shown and described above as being disposed and/or defined by a peripheral surface of the first insulating member 260, in other embodiments, a first insulating member can include one or more slits that are defined by an inner or more central portion of the first insulating member (e.g., a portion corresponding, for example, more closely with a position of a center of an eye of a user. Moreover, while the slits 263 are shown and described above as having a particular nonlinear shape, in other embodiments, a first insulating member can include one or more slits that are linear and/or wedge-shaped. While the first insulating member 260 is shown and described above as being reconfigured such that a single post of the frame 210 (e.g., the posts 238) is inserted through the axially aligned openings 264, in other embodiments, a frame can, for example, include two adjacent posts, each configured to be inserted through a single opening defined by a first insulating member to couple the first insulating member thereto, in a similar manner as described above. Furthermore, while the first insulating member 260 is shown and described above as being coupled to the anterior surface of the frame 210, in other embodiments, at least a portion of a frame can be, for example, inserted into an opening or the like defined by a first insulating member (e.g., the first insulating member can be arranged as a sleeve or the like).

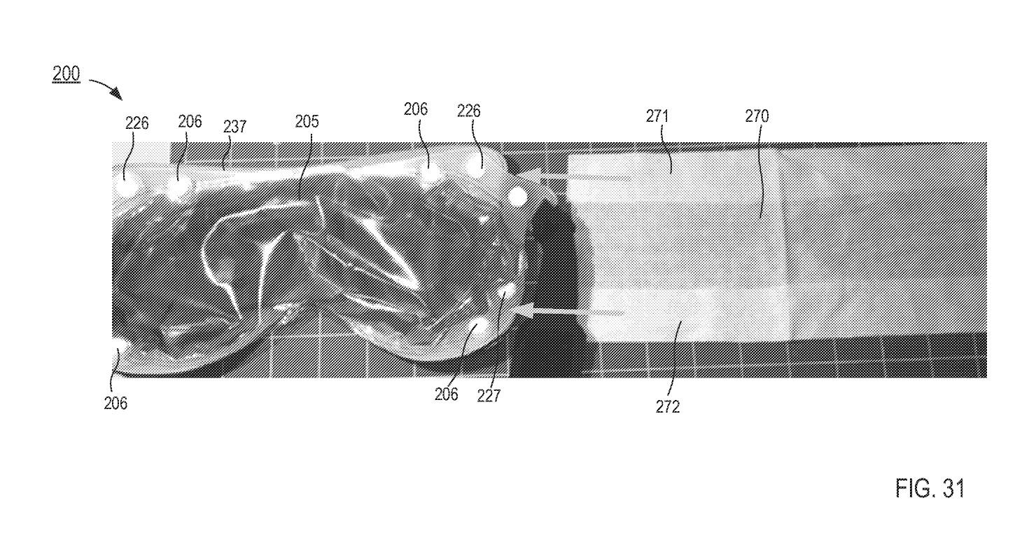

While the first insulating member 260 is described as being disposed in an anterior position relative to the frame 210, the second insulating member 270 can be disposed in a posterior position relative to the therapeutic member 205. For example, as shown in FIGS. 31-33, the second insulating member 270 (also referred to herein, in some embodiments, as “sheet”) can be a relatively thin sheet or fabric that can be coupled to a posterior surface 231 of the frame 210 and/or a posterior surface of the therapeutic member 205. In some embodiments, the second insulating member 270 can be formed from a fibrous nonwoven fabric or the like that can be disposable. In some embodiments, the second insulating member 270 can be substantially similar to the folded sheets described in co-pending U.S. Provisional Patent Application Ser. No. 61/962,067, entitled, “Moistened Nonwoven Disposable Folded Sheets for Use on an Eye Compress, and Methods of Using Same,” filed Oct. 30, 2013, the disclosure of which is incorporated by reference above.

In some embodiments, the second insulating member 270 can include a portion that includes, for example, an adhesive or the like that can couple the second insulating member 270 to the frame 210 and/or the therapeutic member 205. In other embodiments, the second insulating member 270 can be disposed adjacent to the therapeutic member 205 and at least temporarily retained in a fixed position during use of the therapeutic device 200. For example, in use the second insulating member 270 can be disposed between the therapeutic member 205 and the face of the user and can be maintained in a relatively fixed position by a force exerted by the therapeutic device 200 that is operable in coupling the therapeutic device 200 to the user. As another example, the second insulating member 270 can be disposed upon a surface of the therapeutic member 205, and can, for example, be held in place by gravity, moisture, electrostatic force, surface tension, friction, and/or the like.

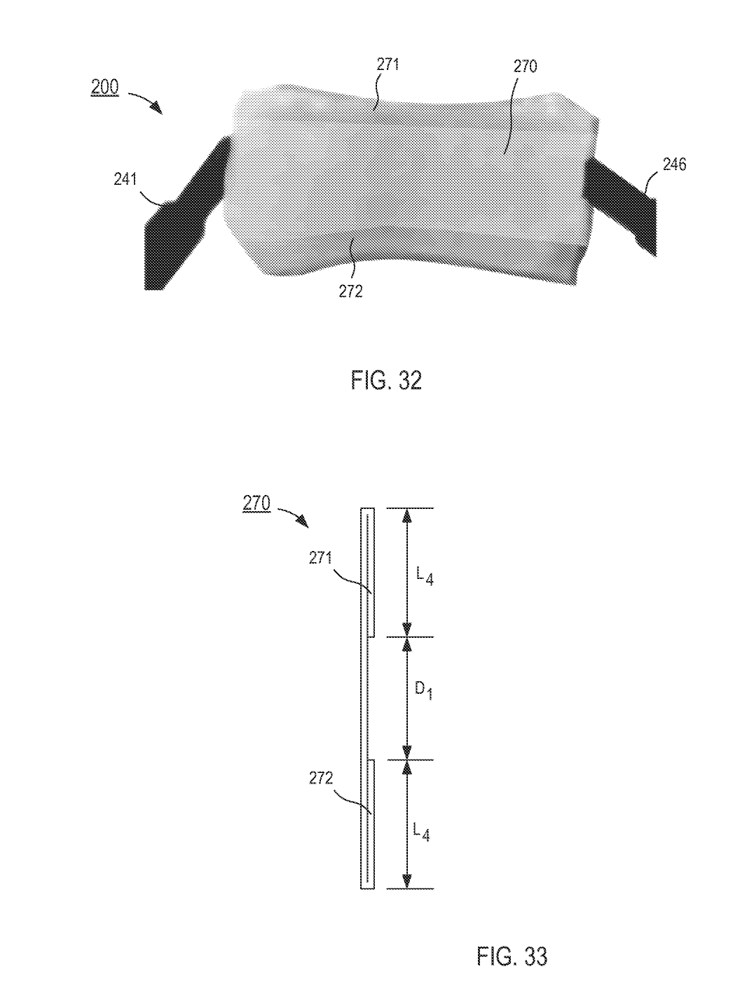

The second insulating member 270 can have a shape and size that is associated with the therapeutic member 205 and/or the frame 210, as shown in FIG. 31. In various embodiments, the second insulating member 270 can be said to have, for example, a perimeter. The perimeter can be defined by the outline of the second insulating member 270. For example, the second insulated member 270, when in a folded configuration, can have a first perimeter, and the second insulated member 270, when in an unfolded configuration, can have a second perimeter that is larger than the first perimeter. As another example, the second insulating portion 270 can have a length and a width that are sufficient to cover at least a portion of the therapeutic member 205 and/or the frame 210 when coupled thereto. For example, the second insulating member 205 can have a length of between about 15.0 and 30.0 cm, between about 17.5 cm and 28 cm, between about 20 cm and 25 cm, between about 21.5 cm and 24 cm or about 23 cm, and a width between about 7.5 cm and 15 cm, between about 8.5 cm and 11.5 cm, between about 9.5 cm and 10.75 cm, or about 10 cm. In some embodiments, the second insulating member 270 can cover substantially the entire therapeutic member 205, as shown in FIG. 32. Moreover, the second insulating member 270 can have any suitable weight and/or density. For example, in some embodiments, the second insulating member 270 can have a density of about 20 grams per square meter (gsm), about 30 gsm, about 40 gsm, about 45 gsm, about 50 gsm, about 55 gsm, about 60 gsm, about 65 gsm, about 70 gsm, about 75 gsm, about 80 gsm, or more and/or any fraction therebetween.

The second insulating member 270 can include a first folded region 271 and a second folded region 272. The first folded region 271 and the second folded region 272 can be, for example, folded in a similar direction such that when the second insulating member 270 is coupled to the therapeutic member 205 and/or the frame 210, the folded regions 271 and 272 are placed in contact with a surface of the therapeutic member 205 and/or the frame 210 (e.g., away from the face, as shown in FIGS. 31 and 32). In other embodiments, the first folded region 271 and the second folded region 272 can be folded in opposite directions from one another (e.g., so that if the first folded region 271 is positioned away from the face, then the second folded region 272 is positioned toward the face, and vice-versa). The folded regions 271 and 272 can be configured to increase a thickness of the second insulating member 270 in selected areas. For example, when coupled to the therapeutic member 205 and/or the frame 210, the first folded region 271 can cover the first attachment points 226 of the frame 210 and a superior set of the couplers 206 of the therapeutic member 205. Similarly, the second folded region 272 can cover the second attachment points 227 of the frame 210 and/or an inferior set of couplers 206 of the therapeutic member 205. For example, as shown in FIG. 33, the first folded region 271 and the second folded region 272 can each have a length L₄ and can define a distance D₁ therebetween. In some embodiments, the distance D₁ and the length L₄ can be substantially the same. In other embodiments, the distance D₁ and the length L₄ can be different (e.g., the distance D₁ is greater than the length L₄). In other embodiments, the first folded region 271 can have a length that is different from a length of the second folded region 272.

By way of example, in some embodiments, the length L₄ of the first folded region 271 and the second folded region 272 can be between about 6.5 cm and 1.25 cm, between about 4.5 cm and 2.0 cm, between about 3.75 cm and 2.5 cm, or about 3.0 cm. The distance D₁ defined between the first folded region 271 and the second folded region 272 can be between about 1.25 cm and 6.5 cm, between about 2.0 cm and 5.75 cm, between about 2.5 cm and 5.0 cm, between about 3.0 cm and 4.5 cm, or about 3.75 cm. In other embodiments, the second insulating member 270 can be arranged such that the distance D₁ is a percentage of the width of the second insulating member 270. By “width of the second insulating member” it is understood that the width can be measured when the second insulating member 270 is either in a folded configuration (wherein the width is equal to, for example, 2*L₄+D₁), or in an unfolded configuration (wherein the width is equal to, for example, 4*L₄+D₁). For example, in some embodiments, the distance D₁ can be about 5%, 6%, 7%, 8%, 9%, 10%, 15%, 20%, 30%, 40%, 50%, 60%, or any suitable percentage or fraction of a percent therebetween of the width of the second insulating member 270. In other embodiments, the second insulating member 270 can be arranged such that distance D₁ is a percentage of the width of the length L₄. For example, the distance D₁ can be about 10%, 15%, 20%, 25%, 50%, 75%, 100%, 120%, 150%, 200%, or any suitable percentage of fraction of a percent therebetween of the length L₄. While in some embodiments, folds in a second insulating member can be prearranged in a package, for example, by a manufacturer, in other embodiments, a second insulating member need not be packaged in a folded configuration. In such embodiments, for example, a user can re-form or fold the second insulating member before or after application of the second insulating member to therapeutic member 205. In some embodiments, the second insulating member can include, for example, a visual indication (e.g., a line, marking, shading, etc.) that can be associated with an axis about which the user can fold the second insulating member.

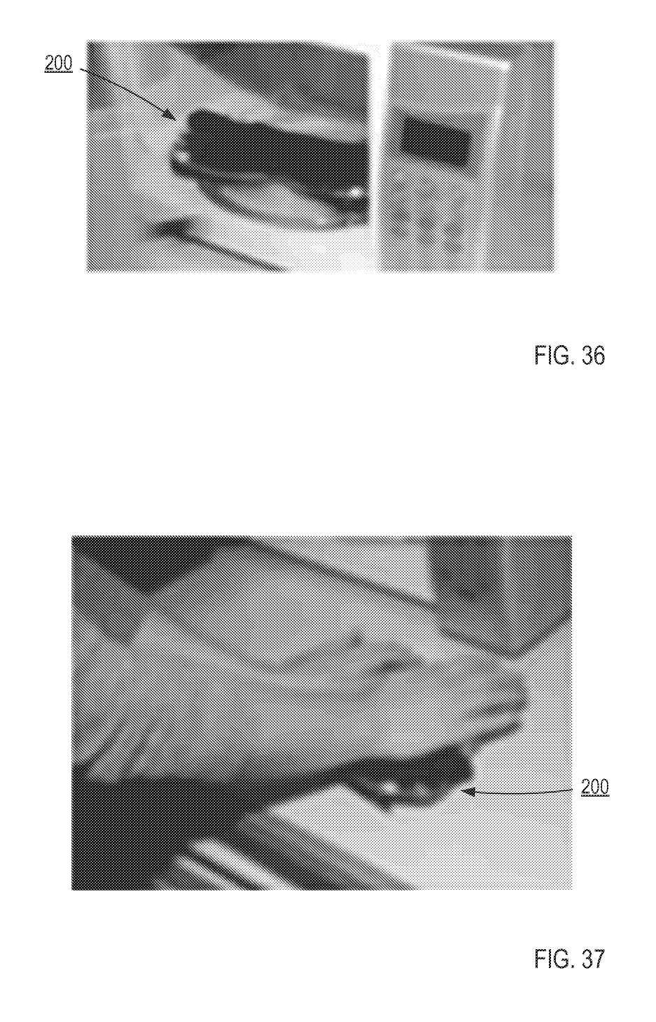

In some instances, the second insulating member 270 can have a moisture content that can enhance the transfer of thermal energy between the ocular region of the user and the therapeutic member 205. For example, as shown in FIG. 36, the user can spray a fluid (e.g., water, saline, etc.) on the second insulating member 270 prior to coupling the therapeutic device 200 to his or her head. Thus, the fluid can enhance the transfer of thermal energy between the ocular region of the user and the therapeutic member 205. Moreover, in some embodiments, the second insulating member 270 can be substantially hydrophilic and/or can include a hydrophilic coating or the like, which can, for example, enhance an interaction between the second insulating member 270 and the fluid. In some embodiments, the fluid can include and/or can otherwise be composed of, for example, an aqueous solvent, a facial botanical extract blend (e.g., Aloe Vera, cucumber extract, and/or the like), 1,3-Dimethylol-5,5-dimethylhydantoin (DMDM hydantoin), iodopropynyl butylcarbamate, propylene glycol, butylene glycol, and any suitable fragrance and/or aromatic agent. Additionally, in some embodiments, the fluid can also include sodium pyrrolidone carboxylate (sodium PCA) and/or sodium lactate, which in some instances, can reduce likelihood and/or severity of skin chapping and/or drying. More specifically, in some embodiments, the fluid can include, for example, propylene glycol having a concentration of between about 0.1% and about 5.0%. In other embodiments, the fluid can have a concentration of propylene glycol of about 0.1%, about 0.25%, about 0.5%, about 0.75%, about 1.0%, about 1.25%, about 1.5%, about 1.75%, about 2%, about 2.25%, about 2.5%, about 2.75%, about 3.0%, about 3.25%, about 3.5%, about 4.0%, about 4.5%, or about 5%. In still other embodiments, the fluid can have a concentration of propylene glycol that is less than about 0.1% or greater than about 5.0%.

In some embodiments, the fluid can have a concentration of butylene glycol of between about 0.05% and about 3.0%. In other embodiments, the fluid can have a concentration of butylene glycol of about 0.1%, about 0.2%, about 0.3%, about 0.4%, about 0.5%, about 0.6%, about 0.7%, about 0.8%, about 0.9%, about 1.0%, about 1.25%, about 1.50%, about 1.75%, about 2.00%, about 2.50%, or about 3.0%. In still other embodiments, the fluid can have a concentration of butylene glycol of less than about 0.05% or greater than about 3.0%.

In some embodiments, the fluid can have a concentration of sodium pyrrolidone carboxylate (sodium PCA) that is similar to or the same as either the concentration of propylene glycol or the concentration of butylene glycol. Similarly, in other embodiments, the fluid can have a concentration of sodium lactate that is similar to or the same as either the concentration of propylene glycol or the concentration of butylene glycol.

In some embodiments, the fluid can have a concentration of fragrance of between about 0.0025% and about 1.5%. In other embodiments, the fluid can have a concentration of fragrance of about 0.0025%, about 0.005%, about 0.0075%, about 0.01%, about 0.0125%, about 0.015%, about 0.02%, about 0.03%, about 0.04%, about 0.05%, about 0.06%, about 0.07%, about 0.08%, about 0.09%, about 0.10%, about 0.12%, about 0.14%, about 0.16%, about 0.18%, about 0.20%, about 0.22%, about 0.24%, about 0.26%, about 0.28%, about 0.30%, about 0.35%, about 0.40%, about 0.45%, about 0.50%, about 0.75%, about 1.0%, about 1.25%, or about 1.5%. In still other embodiments, the fluid can have a concentration of fragrance of less than about 0.0025% or greater than about 1.5%.

In some embodiments, the fluid can have a concentration of a facial botanical extract blend of between about 0.05% and about 3%. In some embodiments, the fluid can have a concentration of a facial botanical extract blend of about 0.1%, about 0.2%, about 0.3%, about 0.4%, about 0.5%, about 0.6%, about 0.7%, about 0.8%, about 0.9%, about 1.0%, about 1.25%, about 1.50%, about 1.75%, about 2.00%, about 2.50%, or about 3.0%. In still other embodiments, the fluid can have a concentration of a facial botanical extract blend of less than about 0.05% or greater than about 3.0%.

The fluid can, in some embodiments, provide greater comfort to the user, by increasing the amount of moisture applied to the user's body. In some embodiments, the fluid can include an aromatic and/or therapeutic agent. Thus, spraying the second insulating member 270 with the fluid (i.e., that includes the aromatic and/or therapeutic agent) can increase the amount of the agent, such as a fragrance, contained in and/or suspended by the second insulating member 270. In some instances, the second insulating member 270 can be selectively sprayed with a fluid in one or more targeted locations along the second insulating member 270 which can be associated with, for example, a specific region and/or a corresponding anatomical structure. Moreover, in some instances, an area of the second insulating member 270 sprayed by a fluid can be increased or decreased by increasing or decreasing, respectively, a distance between the second insulating member 270 and, for example, a spray nozzle of a spray bottle. For example, in some instances, when disposed at a distance of around 0.6 cm from the second insulating member 270, a small mist atomizer of a size typically fitted to a spray bottle of around 60 to 120 ml of fluid can deliver a spray of fluid to a substantially circular area of the second insulating member 270 with a radius of around 2.75 cm. Similarly, in other instances, when such a sprayer is disposed at about 1.25 cm from the second insulating member 270, a substantially circular area with a radius of around 4 cm can be sprayed; when the sprayer is disposed at about 2.5 cm from the second insulating member 270, a substantially circular area with a radius of about 4.5 cm can be sprayed, and when the sprayer is disposed at about 4 cm from the second insulating member 270, a substantially circular area with a radius of about 5 cm can be sprayed.

In some instances in which a second insulating member 270, when disposed in a position of use on the therapeutic member 205 (as described above), covers a surface area of, for example, around 230 square centimeters, a spray radius of about 2.5 cm to 5.0 cm allows for delivery of a focused area of spray delivery, which can be, for example, about 3% to about 5.5% of the surface area of secondary insulating member 270. In this manner, a desired amount of fluid can be sprayed on a focused area of the second insulating member 270 at one or more target locations along the surface area of the second insulating member 270, where each target location can correspond to a particular anatomic region. Because the degree of moisture can furthermore affect the transfer of thermal energy that is transmitted from the therapeutic member 205, through secondary insulating member 270, and to the skin of the user, precise moisture control can, for example, enhance both convenience for the user and therapeutic outcomes of the therapeutic device 200. Although a specific range of spray radii is described above, in other embodiments, a broader or larger area of the secondary insulating member 270 can also be targeted by a spray.

In some instances, a manufacturer can, for example, manufacture a spray bottle (such as described above) and the second insulating member 270. In such instances, the spray bottle, one or more second insulating members 270, and instructions for use can be, for example, shipped and/or packaged together as a kit or shipped and/or packaged independently. For example, as shown in FIG. 37, in some embodiments, the second insulating member 270 can be stored in a sealable package 275. The sealable package 275 can be configured to store any number of second insulating members 270 in a substantially stacked configuration and can at least temporarily maintain a moisture level of the second insulating members 270 when sealed. In some instances, the sealable package 275 can be configured to maintain the second insulating members 270 with a first moisture content (i.e., level) that is lower than a second moisture content, whereby the second moisture content is the content typically recommended for use. In such instances, keeping relatively lower moisture content in the sealable package 275 of the second insulating members 270 can, for example, lower costs of manufacture and shipping, and can in some instances lower the risk of moisture dripping down a face of a user when in use. Such a lower moisture level can also improve the thermal insulation qualities of the second insulating member 270.

As shown in FIG. 37, the sealable package defines an opening 276 and includes a seal member 277. The sealable package 275 can be arranged such that the opening 276 exposes at least one of an edge of the first folded region 271 or the second folded region 272. The opening 276 can be any suitable shape, size, or configuration. For example, as shown, the opening 276 can expose an edge of first folded region 271 and an edge of second folded region 272. In embodiments, opening 276 can have a width W₄ sufficient to expose one or more edges of one or more folded regions (e.g., folded regions 271 and 272). For example, the width W₄ of the opening 276 can be about 3.0 cm, about 3.75 cm, about 4.5 cm, about 5.0 cm, about 5.75 cm, about 6.25 cm, about 7.0 cm, or more, or any fraction therebetween. In one embodiment, the width W₄ of the opening 276 can be about 6.25 cm. In some instances, the width W₄ of the opening 276 can be described as a percentage of the width of the package 275. For example, the width W₄ of the opening 276 can be about 30% of the width of the package 275, about 40% of the width of the package 275, about 50% of the width of the package 275, about 60% of the width of the package 275, or any percent or fraction of a percent therebetween. In some embodiments, the width W₄ of the opening 276 can be less than 30% of the width of the package 275 or can be greater than 60% of the width of the package 275. In one embodiment, the width W₄ of the opening 276 can be about 62.5% of the width of the package 275.

In some embodiments, the package 275 can include and/or be formed from a relatively flexible material and as such, can deform, deflect, and/or otherwise reconfigure when exposed to a force, thereby changing one or more dimensions thereof. Thus, in some instances, the width W₄ of the opening 276 can be described as a percentage of the width of, for example, the second insulating member 270 disposed in the package 275. For example, in some instances, the width W₄ of the opening 276 can be described as a percentage of the folded width of the second insulating member 270 (i.e., (2*L₄+D₁), as described above). In other instances, the width W₄ of the opening 276 can be described as a percentage of the unfolded width of the second insulating member 270 (i.e., (4*L₄+D₁), as described above). By way of example, in some embodiments, the width W₄ of the opening 276 can be about 30% of the folded width of the second insulating member 270, about 40% of the folded width of the second insulating member 270, about 50% of the folded width of the second insulating member 270, about 60% of the folded width of the second insulating member 270, or any percent or fraction of a percent therebetween. In some embodiments, the width W₄ of the opening 276 can be less than 30% of the folded width of the second insulating member 270 or can be greater than 60% of the folded width of the second insulating member 270. In one embodiment, the width W₄ of the opening 276 can be about 62.5% of the folded width of the second insulating member 270.

In some instances, the opening 276 can have and/or can define an area, where the area is a product of the length and the width of the opening 276. In some embodiments, the opening 276 can have an area of about 16 square centimeters (sq. cm.), about 19 sq. cm., about 22.5 sq. cm., about 25.8 sq. cm., about 29 sq. cm., about 30.5 sq. cm., about 32 sq. cm., or any area or fraction of an area therebetween. In some embodiments, the area of the opening 276 can be less than 16 sq. cm. or greater than about 32 sq. cm. In one embodiment, the opening 276 can have and/or can define an area of around 2 sq. cm. Thus, a user can remove the second insulating member 270 by engaging a pick point or the like formed by a folded region, via the opening 276.

Although shown in FIG. 37 in a substantially open configuration, the seal member 277 can be moved relative to the opening 276 to substantially fluidically isolate an inner volume defined by the sealable package 275 from a volume outside of the sealable package 275. For example, in some embodiments, the seal member 277 can include an adhesive or the like that can form the substantially fluidic seal. In other embodiments, the seal member 277 can engage a surface of the sealable package 275 to form a friction or snap fit that can form the substantially fluidic seal. Thus, the second insulating members 270 can be maintained in an environment having suitable moisture content.

Although the second insulating member 270 and the sealable package 275 have been particularly shown and described above, in other embodiments, a second insulating member can have any suitable shape, size, and/or configuration and can be stored in any suitable package prior to use. For example, although the second insulating member 270 is shown and described as including the folded regions 271 and 272, in other embodiments, a second insulating member 270 can be disposed in a package and positioned on the therapeutic member 205 without being folded. In some embodiments, a package configured to store one or more second insulating members can have a size or shape associated with a second insulating member being substantially folded in half (e.g., along its length). Although the package 275 is shown and described above with reference to FIG. 33, as defining the opening 276 in a substantially centered position relative to the package 275 and/or the second insulating members 270 disposed therein, in other embodiments, a package can define an offset or off-center opening that is at least partially aligned with a folded region of a second insulating member disposed therein. Thus, a user can, for example, access one folded region (for example, an upper folded region) of the second insulating member through the opening (e.g., the folded region can be, for example, a pick-point), whereas another folded region (for example, a lower folded region) can be hidden from view from the user behind the casing of the package. In other embodiments, the package can have any suitable shape and/or size and can define an opening through which the user can engage any suitable portion of a second insulating member disposed therein (e.g., no predefined pick-point).

In use, the therapeutic member 205 can be configured to, for example, receive energy, including for example microwave radiation and/or thermal energy. In some embodiments, the therapeutic device 200 can be placed in a microwave oven, disposed in hot water, positioned in a heating device, electrically connected to an electrical source, and/or the like such that the potential thermal energy of the therapeutic member 205 is increased. As shown in FIG. 26, the therapeutic device 200 can be placed in a conventional microwave oven to receive energy that results in an increase of temperature of the therapeutic member 205.

In some embodiments, structured methods of, for example, thermally adjusting and preparing the therapeutic member 205 for use can be followed to attain, for example, a desired outcome. For example, in some embodiments, it can be desirable for a surface of therapeutic member 205 to rapidly attain, and thereafter to sustain for a duration a particular temperature or range of temperatures. Accurate, precise, and/or homogeneous preparation of substantially an entire volume of thermally-activatable substance within a therapeutic member 205 to a desired temperature can result in efficient, predictable, and/or convenient achievement of desired outcomes. In a structured method of preparation involving several variables, for example, targeted incremental changes to a single variable can allow precise control of outcomes.

In some embodiments, for example, a predetermined number of seconds of microwave activation can be applied to the therapeutic member 205. For example, a microwave activation of about 15 seconds can be applied. In certain embodiments, a microwave activation period can be selected based on a particular volume of material in the therapeutic member 205. Thus for example a microwave activation of about 15 seconds can be applied to the therapeutic member 205 containing, for example, approximately 200 grams of thermally activatable gelatinous substance, as described further below. In some embodiments, manipulation of the therapeutic member 205 can take place following a heating step. The therapeutic member 205 can be, for example, massaged, pressed, mixed, kneaded, or otherwise reconfigured to evenly distribute, within its volume, areas of gelatinous or liquid substance having different temperatures. For example, in some embodiments, pressure can be systematically placed upon alternating portions of the therapeutic device 200 and/or therapeutic member 205 in order to achieve a substantially homogeneous distribution of temperature within a malleable, semi-liquid, or gel-like substance. As a specific example, alternating pressure can be applied about 10 times, for a total of about 20 presses, as shown in FIG. 27. The therapeutic member 205 can be configured to receive more thermal energy following an initial increase in thermal energy. For example, in some embodiments, following a first step including microwave oven activation for a desired amount of time followed by a mixing of contents, the therapeutic member 205 can undergo a second microwave oven activation, for example, using the same amount of time as the first microwave oven activation. The therapeutic member 205 can thereafter receive an additional mixing of contents in order to, for example, complete a second step of preparation.

In some instances, a duration of microwave activation can be varied in order to pursue a desired outcome in a predictable manner. Thus, in some instances, if preparation produces a thermal effect that is either below or above a desired temperature, a microwave activation duration can be increased or decreased, respectively, by approximately one or two seconds. For example, if after the two steps of microwave activation and mixing of contents, the temperature of the therapeutic member 205 is found to be insufficiently warm, the timing of microwave activation at a subsequent heating (following a period of time sufficient to allow therapeutic member 205 to attain, for example, room temperature) can be adjusted, for example, by increasing the timing of activation by about one or two seconds. The regimented preparation of therapeutic member 205, consisting of two steps, each step including microwave activation starting at 15 seconds and followed by mixing of contents; as well as a regimented adjustment in which microwave activation timing was adjusted by one or two seconds in order to achieve an intended outcome, was non-arbitrary and was based upon a four-part study, as described below.

In the first part of the study, it was determined through empirical testing that a more thorough distribution of heat in the contents included in the therapeutic member 205 (also referred to herein as “gel contents”) could be obtained through the sequential heating, mixing, re-heating, and re-mixing of the gel contents than could be achieved through a single heating and mixing of the gel contents, even when the total duration of microwave activation and the total amount of mixing remained constant. Said another way, when a single microwave activation (of a first number of seconds and energy setting) followed by a single mixing (of a first number of alternating impressions) was divided into two discrete steps while maintaining the same total length of microwave activation and the same total number of alternating impressions of mixing, respectively, (i.e., each of the two discrete steps consisted of a microwave activation using half the first number of seconds followed by mixing using half the first number of alternating impressions), the thermal homogeneity of the gel contents was increased, relative to the method using a single microwave activation and a single mixing.

In the second part of the study, detailed interviewing with test subjects and dry eye patients regarding instructions for preparing a microwave-heated therapeutic member (e.g., the therapeutic member 205) concluded that both initial comprehension and subsequent recall of directions for use was increased when, for example, the directions for use instructed users to perform two microwave activations using equal numbers of seconds, rather than two microwave activations at somewhat unequal times. A similar finding occurred regarding the number of alternating impressions performed when mixing the therapeutic member after heating. For example, it was found to be easier for users to remember an instruction such as “heat twice, for 10 seconds each time,” or “heat for 10 seconds a first time, and 10 seconds the next time” rather than, for example, “heat for 10 seconds a first time, and heat for 8 seconds the next time.” It is understood that “heating for 10 seconds” is equivalent to “activate in a microwave oven for 10 seconds.” Comprehension and subsequent recall was also improved when “round” numbers (e.g., 10, 15, 20, etc.) were used for both the duration of heating and number of alternating impressions, rather than “non-round” numbers (e.g., 11, 16, 19, etc.). The use of “round” numbers also allowed variations in the adjustment of heating-times mentioned above. For example, instead of increasing a heating-time from, for example, two rounds of 15 seconds to two rounds of 16 seconds or 17 seconds, the use of “round” numbers allowed users to increase a heating time from, for example, two rounds of 15 seconds to one round of 15 seconds and a second round of 20 seconds.

The third part of the study was conducted using numerous standardized therapeutic members of a uniform weight and volume using about 200 grams of gel product, and tested across 4 different microwave ovens. Three heating (microwave activation) and mixing protocols were tested. The protocols tested two steps of heating and mixing, with heating (on a “high” setting) placed at three time settings: 15 seconds, 20 seconds, and 25 seconds. The results were measured using a highly accurate electronic thermometer placed in contact with a surface of the therapeutic member. The results are shown in table 1 below:

TABLE 1

| | | | | Average Temperature | | |
|---|---|---|---|---|---|---|
| | | | | (Centigrade) | | |
| | | | | Achieved with Timing | | |
| Microwave Oven Characteristics | | | | (secs + secs)* | | |
| Brand | Watts | Cu. Cm. | Age (Yrs) | 15 + 15 | 20 + 20 | 25 + 25 |
| Ewave | 1,000 | 6,748 | 9 | 39.4 | 45.6 | 50.6 |
| Emerson | 1,050 | 6,497 | 6 | 39.4 | 47.2 | 52.8 |
| Sharp | 1,000 | 6,858 | 4 | 43.3 | 49.4 | 57.2 |
| Cuisinart | 1,000 | 10,419 | 0.2 | 42.8 | 51.1 | 58.9 |

| | | | |
|---|---|---|---|
| AVERAGE: | 41.1 | 48.3 | 55.0 |

As shown, the use of two steps of 15 seconds of heating produced an average result of 41.1 degrees Centigrade (C), whereas the use of two steps of 20 seconds of heating produced an average result of 48.3 degrees C. In some instances, a desired range of thermal application for eyelid heating is around 42.8 to 45.0 degrees C. Thus, in testing of the numerous 200-gram therapeutic members (e.g., the therapeutic member 205) across 4 microwave ovens, the use of two steps of 15 seconds of heating approached but did not exceed the normal lower bound of the target therapeutic temperature of 41.1 degrees C., whereas the use of two rounds of 20 seconds of heating did exceed the normal lower bound of the target therapeutic temperature of 41.1 degrees C.

In the fourth part of the study, scenarios of microwave-oven heating adjustment were discussed with users and were tested on the therapeutic members used in the third part of the study. It was found that a majority of users were apt to adjust a subsequent timing of microwave-oven activation based on a previous experience with, for example, a prescribed and/or recommended time of activation (i.e., heating), even though the result created a timing of activation that was other than a round number. Said another way, most users found it easy to comprehend and remember a method of use in which two microwave heatings of, for example, 15 seconds each, producing a temperature of the gel contents that was, in some instances, insufficient for user comfort, could be adjusted such that a subsequent set of two microwave heatings of, for example, 16 or 17 seconds each, in which the gel contents were slightly warmer than the previous heating of 15 seconds. It was found that allowing the gel pack to return substantially to room temperature after each set of heatings, and using the same microwave oven, improved the accuracy and reproducibility of the set of heatings, which aided user convenience by improving the efficiency of preparation.

In further testing, it was found that a stepwise, incremental increase in temperature over the course of a set of heatings resulted in, for example, improved safety of the heatings (e.g., reduced the likelihood of overheating that could otherwise be injurious). In some instances, such methods of a step-wise, precisely incremental increase in heating-time between uses of the device could also be applied to other heating methods, such as a method of heating a therapeutic member in hot water. Because adequate heating of a therapeutic member in hot water can take longer than, for example, heating of the therapeutic member in a microwave oven, the step-wise incremental increase in time can be on the order of, for example, 5 or 10 seconds rather than, for example, the 1 or 2 second increase associated with microwave heating. While each of the above methods or steps can add incrementally to improvements in the use of the therapeutic device, the communication of two or more of the methods or steps, in the form of instructions for the sequential performance of such methods or steps, can be made to users. In some instances, it was found that instructions regarding the sequential performance of the methods or steps (described above) enabled 35 users, ranging in age from 24 to 84, rapidly to achieve mastery over the therapeutic member preparation with safe and effective outcomes.

In some instances, the first insulating member 260 can be coupled to the frame 210 after the thermal energy is transferred to the therapeutic member 205 (such as, for example, by the methods described above). In other instances, the first insulating member 260 can be coupled to the frame 210 prior to thermal energy being transferred to the therapeutic member 205. Similarly, the second insulating member 270 can be coupled to the frame 210 and/or the therapeutic member 205 before or after the thermal energy is transferred to the therapeutic member 205. In some instances, the user can spray the second insulating member 270 to increase a moisture content of the second insulating member 270, regardless of an initial hydration level of the second insulating member 270 (such as, for example, a hydration level maintained when the second insulating member 270 is disposed in a sealable package or the like). Similarly, the user can spray the second insulating member 270 to increase a chemical content (e.g., a fragrance, therapeutic agent, or the like) of the second insulating member 270, regardless of an initial chemical content. Said another way, in some instances, the second insulating member 270 can be manufactured and packaged with a first level of moisture content and/or chemical content, and despite the manufactured level of moisture content and/or chemical content spray, the user can spray the second insulating member 270 with a spray that can add to, for example, a moisture content and/or a chemical content according to a user's choosing.

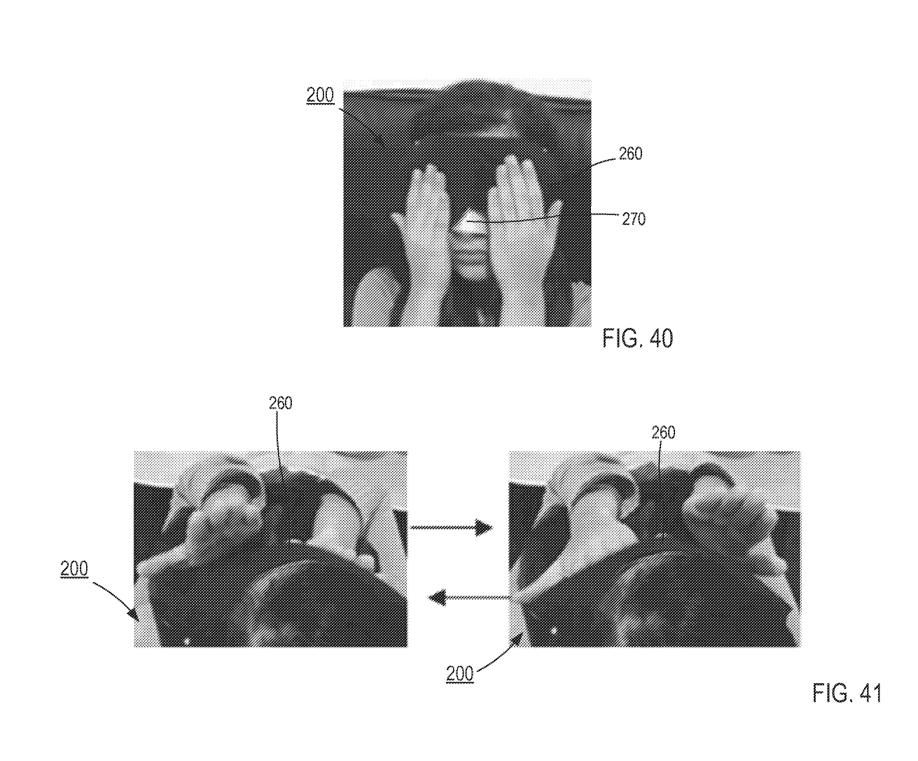

In some instances, at the point of initial contact with the eyes of the user (see, e.g., FIG. 40), the therapeutic device 200 can be maintained in a substantially horizontal rather than a substantially vertical position, allowing, for example, gelatinous contents to be more evenly spread throughout therapeutic member 205 such that substantially all regions of therapeutic member 305 contain roughly equal amounts and/or volumes of the gelatinous contents, thereby creating a substantially symmetric and even application of thermal energy to all areas of the user's face. This method of application is in contrast to a typical manner of applying eyeglasses, eye masks, and other eye apparatus, in which the user brings the apparatus up to the face while maintaining the head in an upright position

Once in the desired position, the coupling portion 240 can be transitioned from its first configuration to its second configuration to couple the therapeutic device 200 to the head of the user. Thus, thermal energy can be transferred from the therapeutic member 205 to the ocular region. Moreover, the arrangement of the therapeutic device 200 can be such that the transfer of the thermal energy is substantially uniform on a desired target area of the ocular region. In addition, the force exerted by the therapeutic member 205 on the ocular region can be distributed and/or diffused in such a manner as to increase the comfort of the user while using the therapeutic device 200. More specifically, the arrangement of the frame 210 can be such that when the therapeutic device 200 is coupled to the head of the user, at least part of the side portion 215 of the frame 210 (e.g., the superotemporal region 216 and/or the centrolateral region 217) can bend, flex, or otherwise reconfigure in such a manner that a force exerted on the ocular region of the user by the therapeutic device 200, or for example by the therapeutic member 205, is reduced. Similarly, the arrangement of the first insulating member 260 in its second configuration is such that the therapeutic member 205 is allowed to extend through the apertures 225 defined by the frame 210 without the first insulating member 260 exerting undue force of the anterior surface of the therapeutic member 205. Thus, the first insulating member 260 can provide thermal insulation without exerting undue force that can otherwise lead to discomfort for the user.

During use of the therapeutic device 200, the thermal energy potential (e.g., a difference in temperature) between the therapeutic member 205 and the ocular region of the user is naturally reduced. In some instances, such a reduction in thermal energy potential can occur in and/or at, for example, peripheral regions of the contents of the therapeutic member 205 that are in closer thermal contact with an outer surface or casing containing the contents at a faster rate than more centrally located regions. More specifically, in some instances, thermal energy can be transferred from the relatively hotter surface of the therapeutic member 205 to an external volume of relatively cooler air via, for example, convection heat transfer. Thermal energy can also be transferred from the relatively hotter surface of the therapeutic member 205 to the external surface of the user's skin via, for example conductive heat transfer, wherein the heat transferred to the user's skin is then dispersed to other areas of the user's body through, for example, the action of the user's blood circulation. In contrast, thermal energy is transferred from more centrally located regions of the contents to more peripherally located regions of the contents substantially via, for example, conduction heat transfer. Thus in some instances, the thermal energy potential between the peripheral regions of the contents of the therapeutic member 205 and the eye region can be reduced at a faster rate than, for example, the thermal energy potential between the central regions of the contents and the eye region.

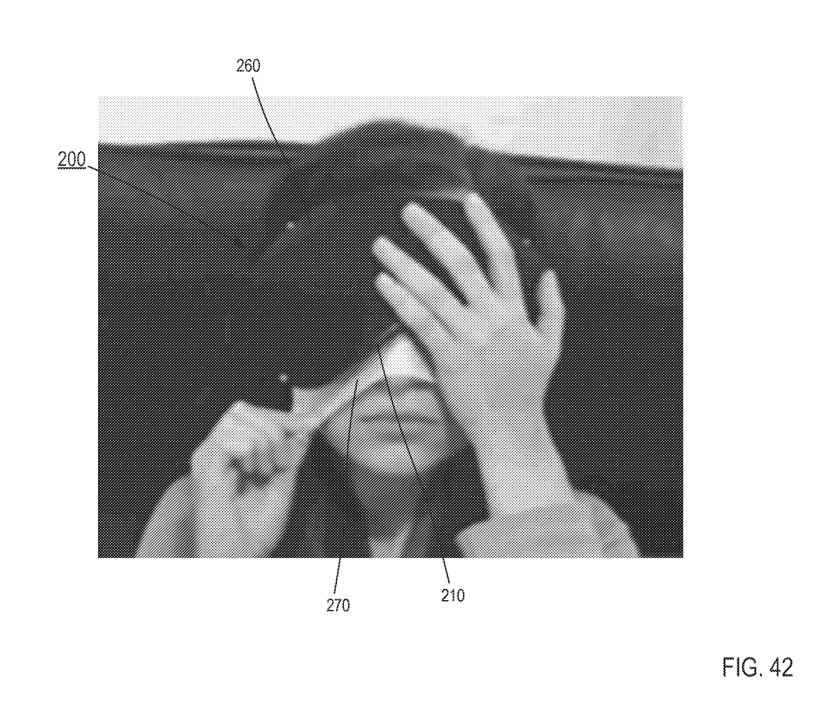

As shown in FIGS. 41 and 42, in some instances, the user can press in an alternating manner upon a portion of the therapeutic device 200 associated with, for example, the left eye and a portion of the therapeutic device 200 associated with, for example, the right eye. In this manner, areas of relatively higher temperature or relatively lower temperature can be diffused in the therapeutic member 205, which in turn, can increase the thermal energy transfer between the therapeutic member 205 and the ocular region of the user. For example, such alternating pressure exerted on the therapeutic member 205 can, in some instances, redistribute the contents of the therapeutic member 205. More specifically, relatively warmer volumes and/or particles of the contents originally disposed in more central locations of the therapeutic member 205 and relatively cooler volumes and/or particles of the contents originally disposed in more peripheral locations of the therapeutic member 205 (as described above) are redistributed within a volume defined by the therapeutic member 205. In this manner, the application of pressure in the alternating manner can, for example, increase a thermal homogeny within the therapeutic member 205. In some instances, such alternating pressure can be modified by the user in order to produce a thermal experience controlled instantaneously by the user. Such a method of use can also extend the user's experience of a useful treatment of thermal application, that might otherwise be inconvenienced by the therapeutic interruption associated with, for example, re-heating the therapeutic member 205 to an acceptable temperature.

In some instances, an action of pressing in an alternating manner upon portions of the therapeutic device 200 can result in pulsating alterations in the pressure transmitted to the ocular regions of the user. Such alterations in pressure can, for example, be transmitted to the eyelid regions and thus, to the meibomian glands, resulting in a form of eyelid treatment that is commonly known as “eyelid massage.” Imparting such alternating differences in pressure can be distinct from or occur simultaneously with the aforementioned diffusion of temperature. Imparting such “eyelid massage” through the content of the therapeutic device 205, such as a gelatinous substance capable of thermal adjustment, can result in a more gentle and safe compression of the meibomian glands than is otherwise available through the more commonly-recommended methods of “eyelid massage,” often employing direct and sometimes uncomfortable and/or unhygienic manipulation of the eyelids with the fingers. In some instances, a synergy of both pressure and temperature application to the eyelids can result from the application of the alternating pressure described above. In some embodiments, the therapeutic member 205 can include and/or can be otherwise packaged with instructions associated with the application of pressure in the alternating manner described above.

As shown in FIG. 42, in some instances, the user can remove the second insulating member 270 from the contact with the therapeutic device 200 and/or the ocular region of the user. Said another way, the user can remove the second insulating member 270 from a position in which insulating member 270 is interposed or sandwiched between therapeutic member 205 and the user's ocular region while therapeutic device 200 is applied to the user's face in a position of use. For example, the user can grasp a portion of the second insulating member 270 and pull the second insulating member 270 from its position between the user and the therapeutic member 205. Thus, the thermal insulation that was provided by the second insulating member 270 is removed, which can result in an increase in thermal energy transfer between the ocular region of the user and the therapeutic member 205. Such a method can be used, for example, once a user has determined that the temperature of treatment has been reduced below a subjective threshold that would ordinarily prompt a user to re-heat the therapeutic member 205. Thus, the removal of the second insulating member 270 can, for example, prolong the thermal therapy to the eye region of the user, during a treatment. In some instances, following further thermal energy transfer from therapeutic device 205 to the user and/or the surrounding environment, the user may again apply a pressure in an alternating manner upon the therapeutic device 200, as discussed above. Once the desired amount of thermal energy is transferred between the ocular region of the user and the therapeutic member 205, the user can engage the coupling portion 240 of the therapeutic device 200 to remove the therapeutic device 200 from the head of the user. For example, the user can move the coupling portion 240 from the second configuration to the first configuration by decoupling the first strap 241 from the second strap 246.

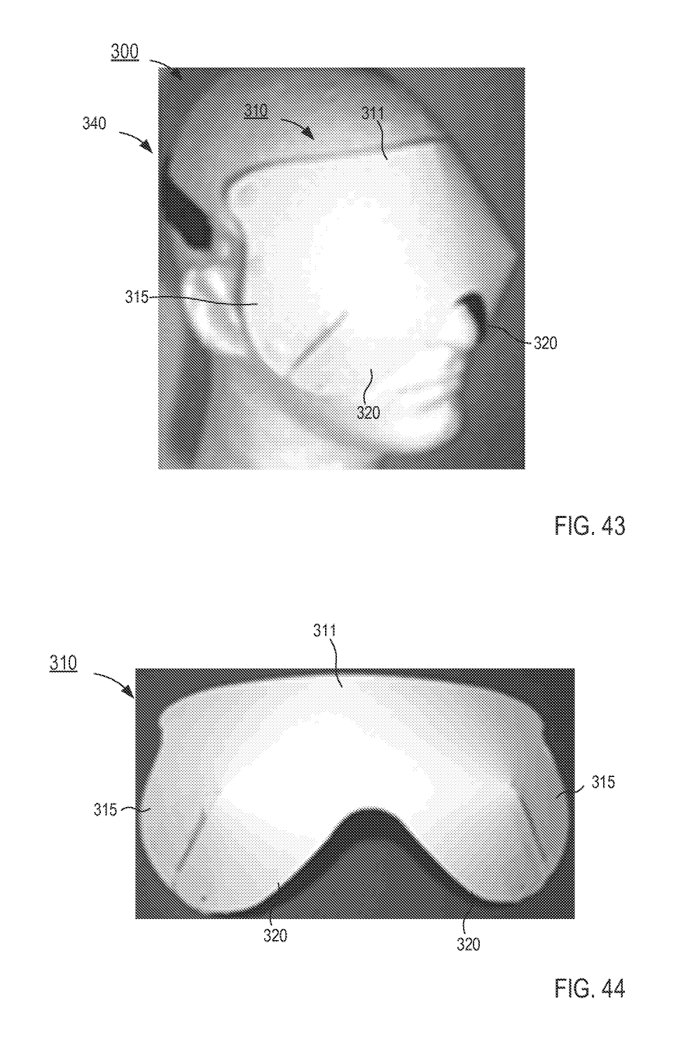

Although the therapeutic device 200 is particularly shown in FIGS. 2-40, a therapeutic device can be any suitable configuration and can include any combination of components that can be similar to the components of the therapeutic device 200. For example, although the flexible frame 210 is shown and described as defining the apertures 225 3 that substantially correspond to the ocular regions (e.g., the left eye and the right eye) of the user, in some embodiments, a therapeutic device can include a frame that does not include apertures. For example, FIGS. 43-47 illustrate a therapeutic device 300 according to another embodiment. The therapeutic device 300 can be used to place a therapeutic member 305 (see e.g., FIG. 37) in contact with a target region of the body of a user such as, for example, the ocular region of the face of the user. The therapeutic device 300 includes a frame 310 and a coupling portion 340. The coupling portion 340 can be substantially similar to or the same as the coupling portion 240 described above with reference to FIGS. 11-21. In this manner, the coupling portion 340 can be coupled to the frame 310 and can be transitioned from a first configuration to a second configuration to couple the therapeutic device 300 to the head of the user (see e.g., FIG. 43), as described in detail above. Thus, the coupling portion 340 is not described in further detail herein.

As shown in FIGS. 44-46, the frame 310 includes a top portion 311, side portions 315, and inferior portions 320. The frame 310 includes and/or forms a pair of convex and/or conical lobes with an apex that is substantially aligned with the globes of the eyes, when the therapeutic device 300 is coupled to the head of the user. In addition, the frame 310 is broadly convex in both a horizontal and a vertical axis. Similarly stated, in addition to the convex form of the lobes, the frame 310 can have a broad convex shape that extends across the full width and height of the eye region covered. As such, the convex form of the frame 310 can reduce direct rearward pressure exerted by the frame 310 on the therapeutic member 305, and hence the globes of the eyes. For example, in some embodiments, the frame 310 can distribute the rearward force on or to peripheral areas of the therapeutic member 305, which in turn, can transmit a rearward force on, for example, the orbital rim, rather than upon the globes of the eyes. In some embodiments, the convex and/or conical lobes diffuse the direct rearward pressure onto a relatively broad area of the orbital rim. In some embodiments, the convex and/or conical lobes diffuse and/or otherwise distribute the direct rearward pressure onto an area of the face of the user that is beyond or outside of the orbital rim. In this manner, the frame 310 can, for example, sandwich, clamp, pin, hold, or otherwise maintain a portion of the therapeutic member 305 between a peripheral portion of the frame 310 and a portion of the face outside of the orbital rim. Moreover, the arrangement of the frame 310 can be such that the rearward force is substantially non-orthogonal to the orbital rim. Similarly stated, the rearward force can be exerted at an angle other than 90° relative to an anterior surface of the orbital rim. In this manner, the rearward force exerted by the therapeutic device 300 on the ocular region can be reduced by, for example, including the frame 310 that forms one or more convex portions, rather than selectively forming the frame with one or more regions of greater flexibility, as described above.

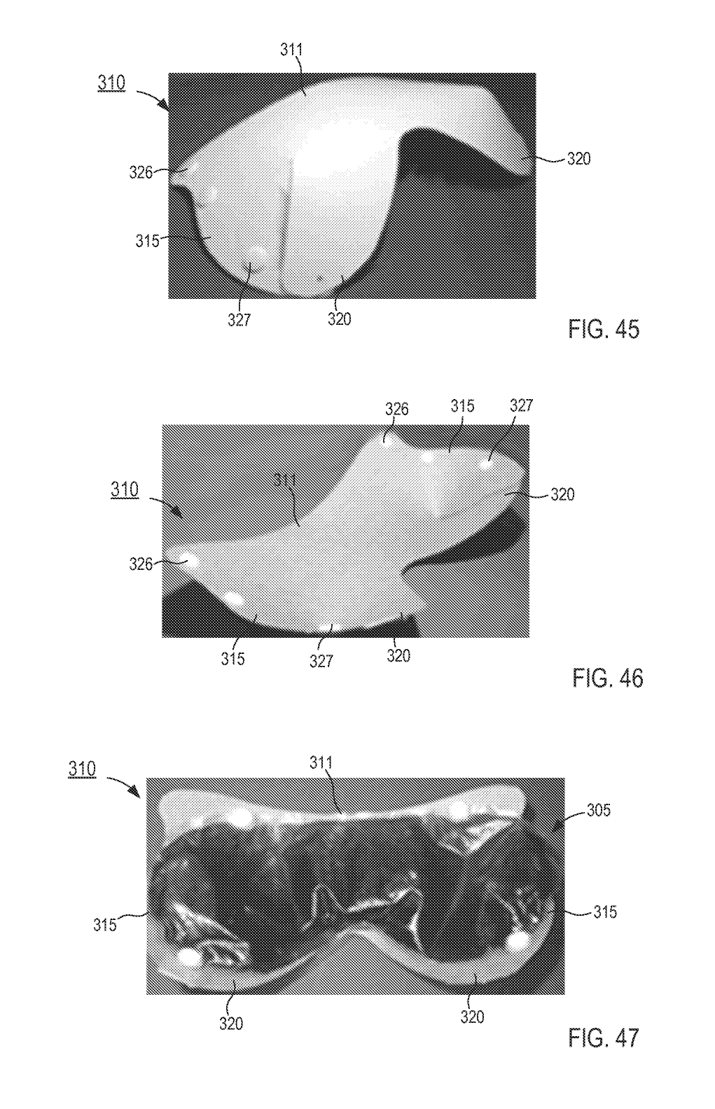

As shown in FIGS. 45 and 46, the frame 310 can include a first set of attachment points 326 and a second set of attachment points 327. As described above with reference to the frame 210, the first set of attachment points 326 and the second set of attachment points 327 can be coupled to, for example, a first strap and a second strap included in the coupling portion 340. Moreover, as shown in FIG. 47, the therapeutic member 305 can be configured to couple to a posterior surface of the frame 310 such that when the therapeutic device 300 is coupled to the head of the user, the therapeutic member 305 is placed in contact with the ocular region.

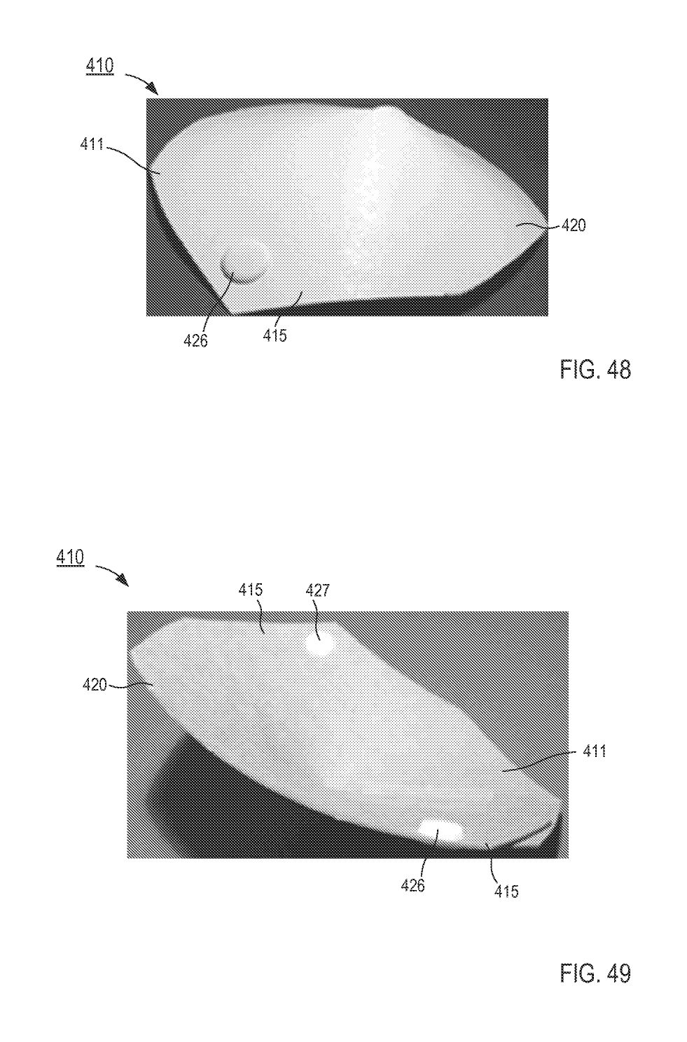

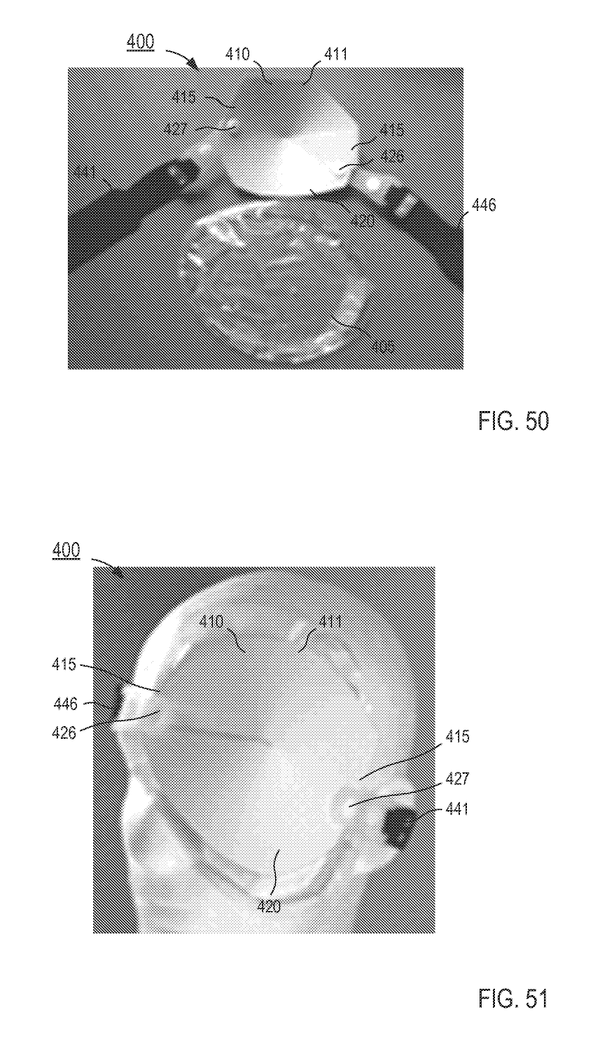

Although the frame 310 is shown and described above with reference to FIGS. 43-47 as including a set of two convex lobes, in other embodiments, a therapeutic device can include a frame with a single convex lobe. For example, FIGS. 48-51 illustrate a therapeutic device 400 according to an embodiment. The therapeutic device 400 can be used to place a therapeutic member 405 (see e.g., FIGS. 50 and 51) in contact with a target region of the body of a user such as, for example, the ocular region of the face of the user. The therapeutic device 400 includes a frame 410 and a coupling portion 440. The coupling portion 440 can be substantially similar to the coupling portion 240 described above with reference to FIGS. 11-21. In this manner, the coupling portion 440 can be coupled to the frame 410 and can be transitioned from a first configuration to a second configuration to couple the therapeutic device 400 to the head of the user (see e.g., FIG. 451), as described in detail above. Thus, the coupling portion 440 is not described in further detail herein.

The frame 410 includes a top portion 411, side portions 415, and an inferior portion 420. The frame 410 includes and/or forms a convex and/or conical lobe with an apex that is substantially aligned with the globe of an eye (e.g., the left or the right eye), when the therapeutic device 400 is coupled to the head of the user. As such, the convex form of the frame 410 can reduce direct rearward pressure exerted by the frame 410 on the therapeutic member 405, and hence the globe of the eye. For example, in some embodiments, the frame 410 can distribute the rearward force on or to peripheral areas on the therapeutic member 405, which in turn can transmit a rearward force on, for example, the orbital rim, rather than upon the globe of the eye. In some embodiments, the convex and/or conical lobe diffuses the direct rearward pressure onto a relatively broad area of the orbital rim. In some embodiments, the convex and/or conical lobe diffuses and/or otherwise distributes the direct rearward pressure onto an area of the face of the user that is beyond or outside of the orbital rim. In this manner, the frame 410 can, for example, sandwich, clamp, pin, hold, or otherwise maintain a portion of the therapeutic member 405 between a peripheral portion of the frame 410 and a portion of the face outside of the orbital rim. Moreover, the arrangement of the frame 410 can be such that the rearward force is substantially non-orthogonal to the orbital rim. Similarly stated, the rearward force can be exerted at an angle other than 90° relative to an anterior surface of the orbital rim.

As shown, the frame 410 includes a first attachment point 426 and a second attachment point 427. The first attachment point 426 and the second attachment point 427 can be coupled to, for example, a first strap and a second strap included in the coupling portion 440. Moreover, as shown in FIG. 41, the therapeutic member 405 can be configured to couple to a posterior surface of the frame 410 such that when the therapeutic device 400 is coupled to the head of the user, the therapeutic member 405 is placed in contact with the ocular region.

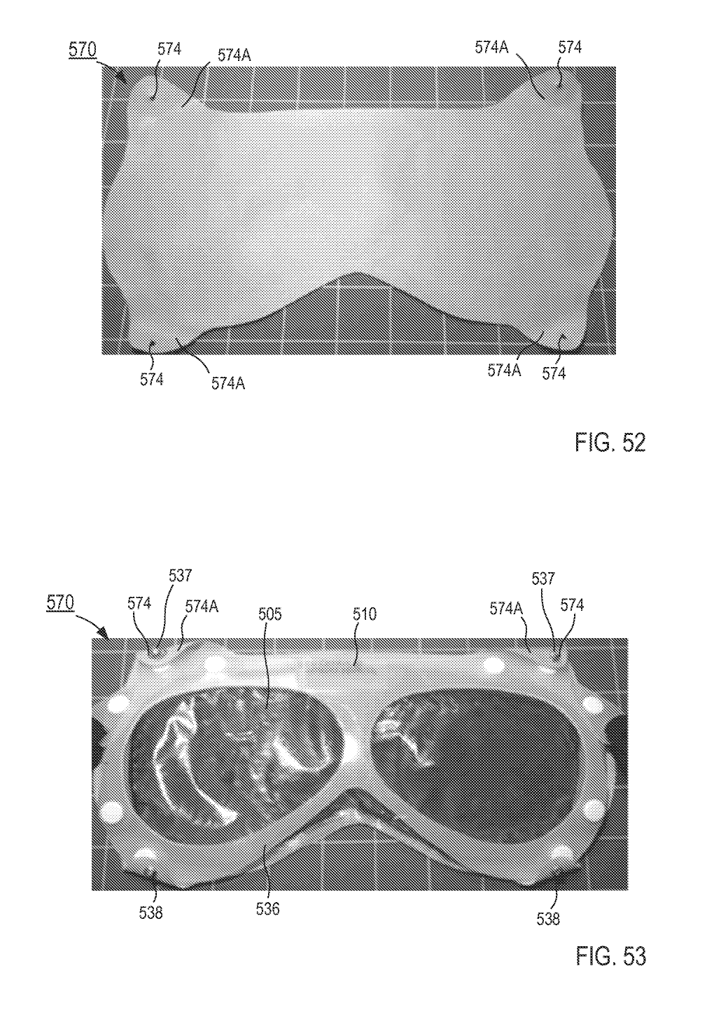

FIGS. 52 and 53 illustrate a second insulating member 570 according to another embodiment. As described in detail below, the second insulating member 570 is configured to be coupled to an anterior surface of a flexible frame 510 (FIG. 53) and/or a posterior surface of a therapeutic member 505 (FIG. 55). More specifically, the flexible frame 510 can be substantially similar to or the same as the flexible frame 210 described above. In this manner, an anterior surface 536 of the flexible frame 537 can include a set of posts 537 that can be operable in coupling the second insulating member 570 to the flexible frame 510. For example, as shown in FIGS. 52 and 53, the second insulating member 570 includes a set of coupling portions 574A that each defines an opening 574. As shown in FIG. 89, the coupling portions 574A can be wrapped around the flexible frame 510 when the second insulating member 570 is disposed adjacent to the posterior surface of the therapeutic member 505. In this manner, the posts 537 can be inserted into the corresponding opening 574 defined by the coupling portions 574A to at least temporarily couple the second insulating member 570 to the flexible frame 510. In some embodiments, the posts 537 can be substantially similar to, for example, posts 237 and 238 of flexible frame 210. As such, in some embodiments, a first insulating member such as the first insulating member 50 and the second insulating member 570 can be coupled to posts 537 and 538.

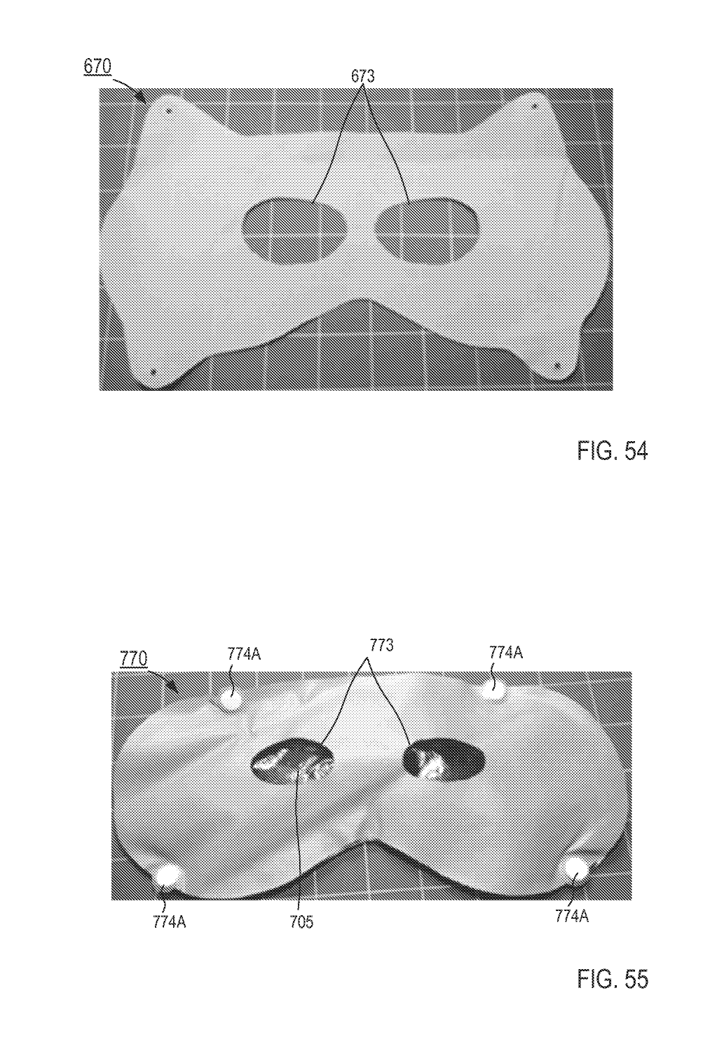

In some embodiments, a second insulating member can be arranged so as not to cover, obscure, or conceal a patient's eyes, from an anterior anatomic perspective, when the second insulating member is positioned adjacent to the face of the user. For example, as shown in FIG. 54 a second insulating member 670, which is otherwise similar to the second insulating member 570, can define a set of apertures 673. The arrangement of the second insulating member 670 is such that the apertures 673 are substantially aligned with, for example, the eyes of a user when the second insulating member 670 is disposed adjacent to the face of the user. Thus, the user's eyes are unobstructed from an anterior anatomic perspective by the second insulating member 670. Said another way, the apertures 673 defined by the second insulating member 670 can be such that an edge of the second insulating member 670 defining each aperture 673 circumscribes at least a portion of an eye of the user, from an anatomic perspective, when the second insulating member 670 is disposed adjacent to the face of the user.

Although the coupling portions 574A of the second insulating member 570 are shown and described as wrapping around a portion of the flexible frame 510 to be coupled to the anterior surface 536, in other embodiments, a second insulating member 570 can include any suitable coupling portion or mechanism. For example, FIG. 55 illustrates a second insulating member 770 coupled to a therapeutic member 705 according to another embodiment. The second insulating member 770 can have a shape that is associated with and/or substantially corresponds to a shape of the therapeutic member 705. As shown, the second insulating member 705 includes a set of couplers 774A that are configured to couple the second insulating member 770 to the therapeutic member 705. In some embodiments, the couplers 774A can be, for example, snaps or the like that can form a snap fit with a corresponding portion of the therapeutic member 705. In this manner, the second insulating member 770 can be removably coupled to the therapeutic member 705.

In other embodiments, the therapeutic member 705 and the second insulating member 770 can be fixedly coupled together via, for example, the couplers 774A. For example, the couplers 774A can be snaps that each include a snap cap and a male or female portion of a snap fixture. In this manner, a portion of therapeutic member 705 and a portion of second insulating member 770 can be disposed (e.g., sandwiched) between the snap cap and the male or female (stud or socket, respectively) portion of the snap fixture. Such a structure can be assembled using a snap press operation wherein a protruding portion of the snap cap passes through the portion of the second insulating member 770, the portion of the therapeutic member 705, and the stud or socket portion in order to secure the assembly in place. In some embodiments, such sandwiching allows the exposed male or female portions (which appear on the side of the therapeutic member which is not covered by the second insulating member 770) to be coupled to a corresponding female or male snap fixture on flexible frame 510 (as described above with reference to the therapeutic member 205 and the flexible frame 210). Thus, the second insulating member 770 can be at least temporarily retained in a fixed position relative to the therapeutic member 705. Moreover, the second insulating member 770 can define a set of apertures 773 that can be substantially aligned with the eyes of a user when the second insulating member 770 is disposed adjacent to the face of the user, as described above.

In some instances, the arrangement of the second insulating members 570, 670, and 770 can be such that, when coupled to a corresponding therapeutic device (as described above), a user can use the therapeutic device without manually affixing a different second insulating member to the therapeutic device prior to use (e.g., after an initial coupling associated with, for example, a first use). The second insulating members 570, 670, and/or 770, either by themselves or coupled to a therapeutic member, can be provided to and used by a user who, for example, already owns and/or already has access to a flexible frame, without the need for purchasing an additional corresponding flexible frame. That is to say, the second insulating members 570, 670, and/or 770 can be manufactured, shipped, purchased, and/or obtained independently of the flexible frame and/or other portions of a therapeutic device.

While referred to herein as the second insulating members 270, 570, 670, and 770 are specifically described herein, in other embodiments, the second insulating members and/or portions thereof can serve different functions. In addition, in some embodiments, a moist sheet structure such as, for example, the secondary insulating member 270 can be disposed on the rear surface (facing the user) of, for example, the second insulating member 670. In some instances, the second insulating member 270 can be disposed between a second insulating member such as, for example, the second insulating member 670 and a therapeutic member such as, for example, the therapeutic member 205, 505 and/or 705. In such instances, a relatively moist heat can be applied, for example, specifically to the periocular region (e.g., via the apertures 673), while avoiding the application of moisture to areas surrounding the periocular region. While the secondary insulating members 670 and 770 are each shown in FIGS. 54 and 55, as having two apertures 673 and 773, respectively, that correspond to a first and a second eye of a user, in other embodiments, a second insulating member can have a single aperture corresponding to a single eye of a user. As a particular example, such a second insulating member can be so configured even when the secondary insulating member is configured to span over both a first and a second eye of a user. In such embodiments, the second insulating member having a single aperture can be used, for example, when treating a primarily or entirely unilateral eye condition, such as an acute chalazion, hordeolum, or the like. In some embodiments, a second insulating member such as for example the second insulating member 670 could be configured to be used alternatively for either a left eye or a right eye, thus reducing inconvenience and cost for the user.

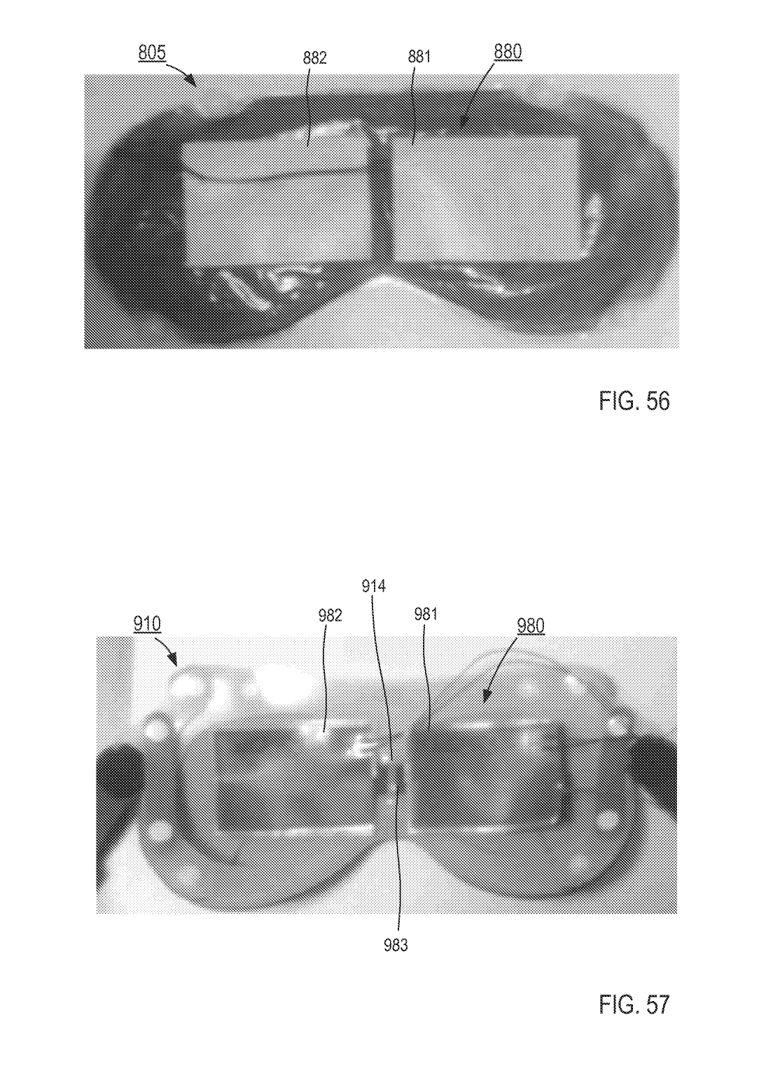

Any of the therapeutic devices and/or components thereof can be used with any suitable device, mechanism, system, circuit, and/or the like that is configured to transfer energy to a therapeutic member included therein. For example, while the therapeutic device 200 is shown and described as being placed in a microwave to increase the energy potential of the therapeutic member 205 (e.g., increase a temperature of a thermal gel or the like include therein), the therapeutic member 205 can receive energy from any suitable source. By way of example, FIG. 56 illustrates a heating mechanism 880 in contact with a therapeutic member 850 according to an embodiment. A heating mechanism can have any number of components. As shown, for example, the heating mechanism 880 includes a first heating member 881 and a second heating member 882. Each heating member 881 and 882 can be electrically coupled to a current source (e.g., a battery, wall outlet, and/or other electrical source). In this manner, the first heating member 881 and the second heating member 882 can receive a flow of current that can be operable in increasing a temperature of at least a portion of the heating members 881 and 882. The arrangement of the heating mechanism 880 is such that the first heating member 881 and the second heating member 882 can each independently heat a portion of the therapeutic member 805. Similarly stated, the first heating member 881 and the second heating member 882 transfer thermal energy to independent and/or targeted portions of the therapeutic member 805. In some embodiments, the portions of the therapeutic member 805 can substantially correspond to, for example, portions of the ocular region of a user. Moreover, with the therapeutic member 805 defining a single inner volume (as described above), the therapeutic member 805 can diffuse and/or distribute the thermal energy to areas and/or portions having a lower thermal energy. Thus, thermal energy can be transferred to the therapeutic member 805 which, in turn, can transfer the thermal energy to, for example, the ocular region of the user. In some embodiments, the heating mechanism 880 can be configured to remain in contact with the therapeutic member 805 during a therapeutic treatment of a user. Therefore, the heating mechanism 880 can supply thermal energy substantially continuously during the therapeutic treatment. In some embodiments, the heating mechanism 880 can be manipulated to adjust the amount of thermal energy transferred to the therapeutic member 805 (e.g., increase or decrease a temperature).

The heating members 881 and 882 can be activated according to similar or dissimilar mechanisms or algorithms, such that the thermal energy delivered by each heating member 881 and 882 at a given point can be equivalent or inequivalent. Similarly, the target or “set-point” temperature for each heating element 881 and 882 can be equivalent or inequivalent at any given point in time. As such, the heating mechanism 880 can be used, for example, to deliver a different thermal application to each eye region of a user. In some embodiments, the heating members 881 and 882 can be, for example, flexible heaters, such as flexible polyimide heating elements and/or the like. The heating members 881 and 882 can be coupled to an outer surface of the therapeutic member 805 that faces away from the eye region of a user when therapeutic member 805 is positioned against the eye region of the user (i.e., positioned on a surface of the therapeutic member 805 that is substantially opposite the surface in contact with the eye region).

The heating mechanism 880 can have any suitable surface area formed from, for example, the sum of the surface areas of the heating members 881 and 882. For example, in some embodiments, the heating members 881 and 882 can each have a surface area of, for example, about 23 square centimeters (sq. cm.), about 27.5 sq. cm., about 33 sq. cm., about 39.5 sq. cm., about 47 sq. cm., about 55 sq. cm., about, sq. cm., about 65 sq. cm., or any surface area or fraction of a surface area therebetween. In other embodiments, the heating members 881 and 882 can each have a surface area less than about 23 sq. cm. or greater than about 65 sq. cm. Moreover, the heating members 881 and 882 can have substantially equivalent or inequivalent surface areas.

In some embodiments, the surface area of the heating mechanism 880 (i.e., the sum of the surface areas of the heating members 881 and 882) can be based at least partially on a surface area of a side (i.e., surface) of the therapeutic member 805 that is placed in contact with the user. For example, in some embodiments, the surface area of heating mechanism 880 can be a percentage of the surface area of the side such as, for example, about 10%, about 20%, about 30%, about 35%, about 40%, about 50%, or any percent or fraction of a percent therebetween. In other embodiments, the surface area of the heating mechanism 880 can be less than about 10% of the surface area of the side or greater than about 50% of the surface area of the surface of the therapeutic member 805. In one embodiment, the surface area of heating mechanism 880 can be about 35% of the surface area of the side of therapeutic member 805.

In some instances, the surface area of the heating mechanism 880 can be at least partially based on, for example, a ratio of the surface area of the heating mechanism 880 to the volume of the contents of therapeutic member 805 (in cubic centimeters (cc)). For example, in some embodiments, the ratio can be about 65 sq. mm. per cc, about 75 sq. mm per cc, about 85 sq. mm per cc, about 95 sq. mm. per cc, about 105 sq. mm. per cc, about 115 sq. mm per cc, about 125 sq. mm per cc, about 135 sq. mm per cc, about 150 sq. mm per cc, about 165 sq. mm per cc, or any ratio or fraction of a ratio therebetween. In other embodiments, the surface area-to-volume ratio can be less than about 65 sq. mm per cc or greater than about 165 sq. mm per cc. In one embodiment, the surface area-to-volume ratio can be about 125 sq. mm per cc.

Similarly, the heating mechanism 880 can be configured to have, for example, a wattage-to-volume ratio, wherein the wattage of heating mechanism 880 can be the sum of the products of the wattage rating and the surface area of each heating element 881 and 882 and the volume is the volume of the therapeutic member 805. For example, in some embodiments, the wattage-to-volume ratio of heating mechanism 880 can be about 0.1 watt per cc, about 0.2 watt per cc, about 0.25 watt per cc, about 0.3 watt per cc, about 0.35 watt per cc, about 0.4 watt per cc, about 0.45 watt per cc, about 0.5 watts per cc, or any ratio or fraction of a ratio therebetween. In other embodiments, the wattage-to-volume ratio can be less than about 0.1 watt per cc or greater than about 0.5 watt per cc. In one embodiment, the wattage-to-volume ratio can be about 0.3 watts per cc.

While the heating mechanism 880 is shown in FIG. 56 as being applied to therapeutic member 805 without the presence of a surrounding enclosure, in other embodiments, a heating mechanism can be activated while a therapeutic member is enclosed in a surrounding enclosure such as, for example, an insulated container. In such embodiments, disposing a therapeutic member (e.g., the therapeutic member 805) in an insulated container can, for example, limit and/or minimize convective or conductive heat loss, which can improve heating efficiency and/or reduce a time of heating. In some instances, once a desired amount of thermal energy has been transferred to the therapeutic member 805, the therapeutic member 805 can be removed from the container and applied to the eye region of a user (as described above). In some instances, the heating mechanism 880 can continue to supply thermal energy to the therapeutic member 805 during use (e.g., after the therapeutic member 805 is removed from the insulated container and applied to the eye region of the user to, for example, substantially maintain a desired temperature.

While the heating mechanism is shown in FIG. 56 as being directly coupled to the therapeutic member, in other embodiments, a heating mechanism can be coupled to a flexible frame of a therapeutic device. For example, FIG. 57 illustrates a heating mechanism 980 that is coupled to a flexible frame 910 according to another embodiment. The heating mechanism 980 includes a first heating member 981 and a second heating member 982 that can be substantially similar to the first heating member 881 and the second heating member 882 described above. Furthermore, the heating mechanism 980 includes a coupling portion 983 that can engage a coupling portion 914 of the flexible frame 910. The arrangement of the coupling portions 914 and 983 can be such that the first heating member 981 and the second heating member 982 are at least temporarily maintained in contact with a therapeutic member (not shown in FIG. 57) coupled to the flexible frame 910. Thus, thermal energy can be transferred to the therapeutic member, as described above.

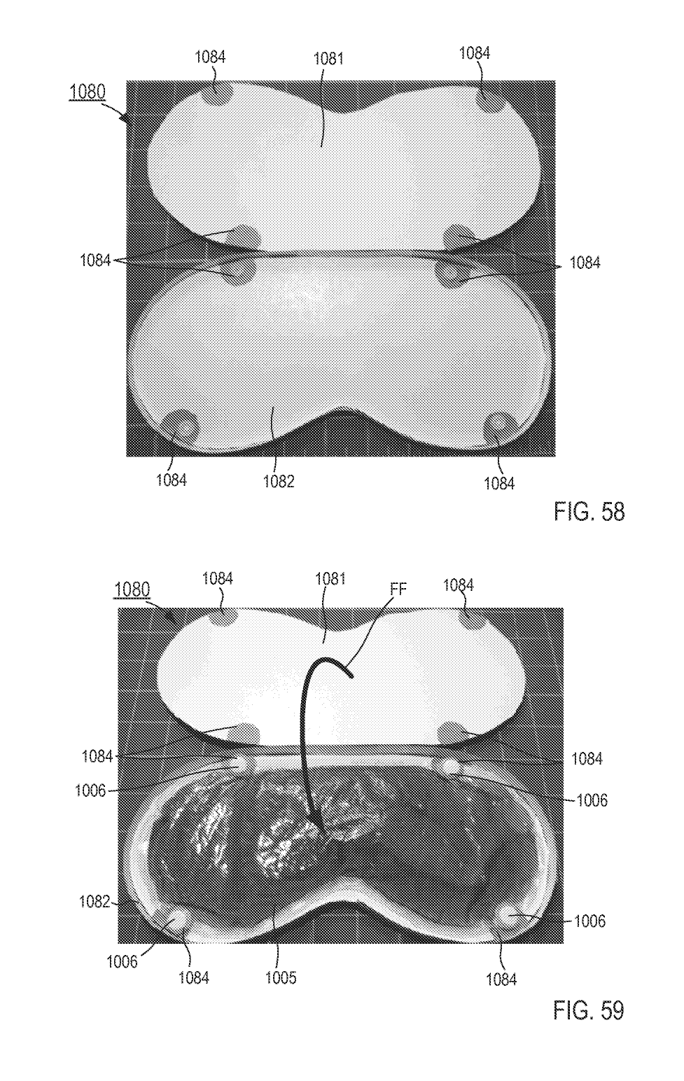

Although the first heating members 881 and 981 and the second heating members 882 and 982 are shown as being disposed on the same side of a therapeutic device, in other embodiments, a heating mechanism can be configured to substantially surround a therapeutic member. For example, FIGS. 58 and 59 illustrate a heating mechanism 1080 according to an embodiment. The heating mechanism 1080 includes a first heating member 1081 and a second heating member 1082 that can be moved relative to one another to define an inner volume configured to receive a therapeutic member 1005. For example, a first surface of the therapeutic member 1005 can be placed in contact with the second heating member 1082 and the first heating mechanism 1081 can be moved relative to the second heating member 1082 to contact a second surface of the therapeutic member 1005, opposite the first surface, as indicated by the arrow FF in FIG. 59. In this manner, the heating mechanism 1080 can receive a flow of current that is operable in increasing a temperature of at least a portion of the heating members 1081 and 1082, and thus, the therapeutic member 1005 disposed therebetween. Moreover, as shown the first heating member 1081 and the second heating member 1082 can include a set of insulators 1084 that are configured to insulate a portion of the heating members 1081 and 1082, thereby reducing the amount of thermal energy transferred thereto. As shown in FIG. 59, in some embodiments, the heating mechanism 1080 can be arranged such that the insulators 1084 are substantially aligned with a set of couplers 1006 (e.g., similar to the couplers 206 of the therapeutic member 205) included in the therapeutic member 1005. Thus, the insulators 1084 can reduce the amount of thermal energy transferred to the couplers 1006 that can otherwise result in discomfort when in contact with the user. In some embodiments, insulators 1084 can be configured to matingly couple with couplers 1006 in order to, for example, form a more stable positioning of therapeutic member 1005 relative to the heating members 1081 and 1082.

In other embodiments, a heating mechanism configured to substantially surround a therapeutic member can have a single heating member, or can have heating members with substantially dissimilar heating profiles. For example, in some embodiments, the heating member 1081 can be an insulating member or a heating member with a low thermal energy output, whereas the heating member 1082 can be a heating member with, for example, a relatively large thermal energy output, such that heating of the therapeutic member 805 is primarily or exclusively via the heating member 1082. In this manner, thermal energy can be transferred to the therapeutic member 805 by the heating member 1082, while the thermocouple can determine and/or sense a temperature associated with the therapeutic member 805. In some instances, the thermocouple can be configured to send a signal to the heating member 1082, for example, when a desired temperature or “set-point” has been reached, and, upon receipt, the heating member 1083 can be configured to substantially stop the transfer of thermal energy to the therapeutic member 805. In some instances, disposing the thermocouple on a side of the therapeutic member 805 that is opposite a side in contact with the heating mechanism 1080 can ensure a heating throughout substantially the entire volume of the therapeutic member 805.

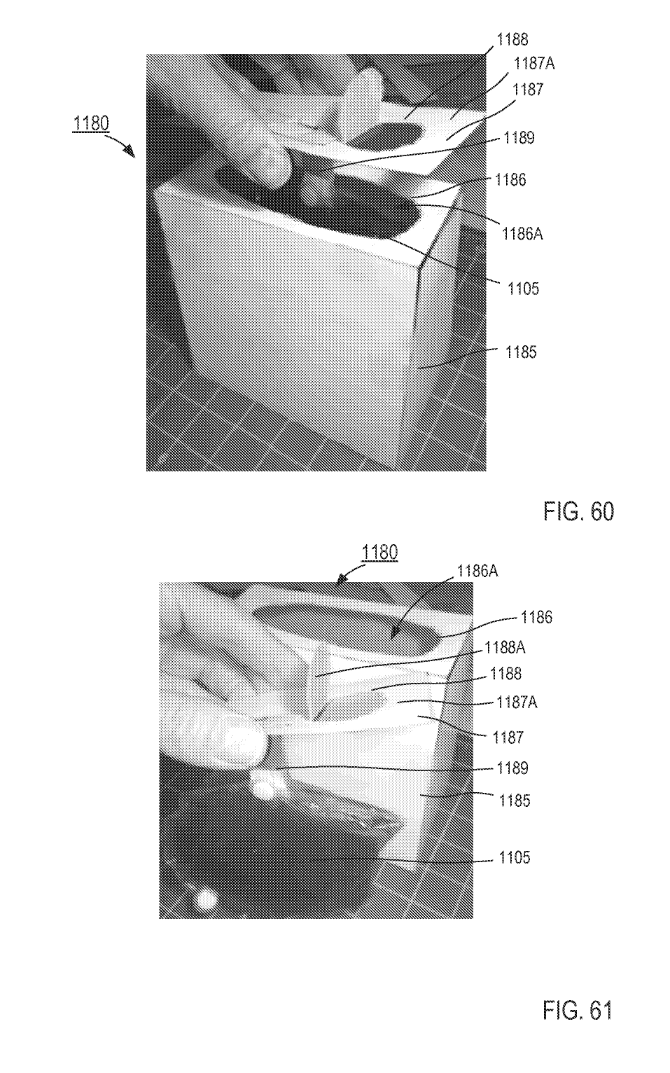

In still other embodiments, a therapeutic member can receive thermal energy by being at least partially disposed, for example, in a warm bath of a fluid (e.g., water). For example, FIGS. 60-62 illustrate a heating mechanism 1180 according to another embodiment. The heating mechanism 1180 includes a reservoir 1185 and a hanging mechanism 1187. The reservoir 1185 includes a top surface that defines an opening 1186 configured to allow access to an inner volume 1186A of the reservoir 1185, as shown in FIGS. 60 and 61. The reservoir 1185 can be any suitable shape, size, or configuration. For example, in some embodiments, the reservoir 1185 can be formed from and/or can include an insulating material or the like that can, for example, limit a transfer of thermal energy from the inner volume 1186A to a volume substantially outside of the reservoir 1185.

The hanging mechanism 1187 can be any suitable configuration and is configured to be at least partially disposed in the inner volume 1186A of the reservoir 1185, as described in further detail herein. The hanging mechanism 1187 includes a base 1187A and a hanger 1189. The base 1187A can be any suitable shape, size, or configuration. For example, in some embodiments, the base 1187A can have a size and a shape that substantially corresponds with and/or is otherwise associated with the top surface of the reservoir 1185. In this manner, the base 1187A can be disposed on and/or positioned adjacent to the top surface of the reservoir 1185 to insert the hanger 1189 through the opening 1186 such that a portion of the hanger 1189 is disposed in the inner volume 1186A of the reservoir 1185. As shown, the base 1187A defines an opening 1188 that can, for example, allow access to the inner volume 1186A of the reservoir 1185 when the base 1187A is disposed on and/or adjacent to the top surface of the reservoir 1185, as described in further detail herein.

As shown in FIGS. 60-68, the hanging mechanism 1187 is configured to be coupled to a therapeutic member 1105. The therapeutic member 1105 can be any suitable member such as any of those described herein (e.g., the therapeutic member 1105 can be substantially similar to or the same as the therapeutic member 205 described in detail above). More specifically, the hanger 1189 extends from a surface of the base 1187A and has an end portion configured to be coupled to the therapeutic member 1105. For example, in some embodiments, the end portion of the hanger 1189 can include a coupling mechanism such as, for example, a snap and/or the like that can be matingly coupled to a corresponding coupling mechanism (e.g., a corresponding snap) of the therapeutic member 1105. As such, the therapeutic member 1105 can be removably coupled to the hanger 1189. Moreover, with the hanger 1189 coupled to the therapeutic member 1105, the therapeutic member 1105 can be disposed in the inner volume 1186A of the reservoir 1185 when the base 1187A of the hanging mechanism 1187 is positioned on and/or adjacent to the top surface of the reservoir 1185.

For example, in use, a patient and/or user can couple the therapeutic member 1105 to the end portion of the hanger 1189, as shown in FIGS. 60-62. Once coupled, the user can move the hanging mechanism 1187 to a position that places the base 1187A on and/or otherwise adjacent to the top surface of the reservoir 1185. As such, the therapeutic member 1105 and the hanger 1189 can be inserted through the opening 1186 to be disposed in the inner volume 1186A of the reservoir 1185. With the base 1187A disposed on and/or adjacent to the top surface, the user can, for example, move a lid or the like to expose the opening 1188 of the base 1187A. In this manner, the opening 1188 can allow access to the inner volume 1186A of the reservoir 1185 (e.g., via the opening 1186).

With the opening 1188 exposed, the user can, for example, pour a fluid into the opening 1188 of the hanging mechanism 1187 and the opening 1186 of the reservoir 1185 and into the inner volume 1186A. In some instances, the fluid can be a predetermined volume of the fluid that is preheated to a desired temperature. For example, in some instances, the fluid can have a volume of about 500 milliliters (ml) and can be heated substantially to the boiling point of the fluid. In this manner, the therapeutic member 1105 can be immersed in the volume of the fluid, which in turn, can transfer a portion of thermal energy from the fluid to the therapeutic member 1105. In some embodiments, the therapeutic member 1105 can be immersed in the fluid for a predetermined time period. For example, in some instances, the therapeutic member 1105 can be immersed in about 500 ml of boiling-hot water for about 60 seconds, which can heat, for example, the contents of the therapeutic member 1105 (e.g., approximately 200 grams of thermal gel, as described in detail above) to approximately 49 C. Thus, after the predetermined time period and/or after a desired amount of thermal energy has been transferred to the therapeutic member 1105, the hanging mechanism 1187 can be moved relative to the reservoir 1185 to remove the therapeutic member 1105 from the inner volume 1186A. In this manner, the therapeutic member 1105 can be decoupled from the hanger 1189 and coupled to a therapeutic device (not shown in FIGS. 60-62), such as those described herein.

Although described above as pouring a preheated volume of fluid into the inner volume 1186A of the reservoir 1185, in other embodiments, a volume of non-heated fluid can be poured into the inner volume 1186A and heated, for example, by a portion of the reservoir 1185. For example, in some embodiments, the reservoir 1185 can include a heating element and/or the like that can be heated (e.g., via a flow of electrical current and/or the like) to transfer thermal energy to the volume of fluid. Although the volume of fluid is specifically described, for example, as being about 500 ml, in other embodiments, any suitable volume of fluid can be poured into the inner volume 1186A (e.g., a volume that is less than the inner volume to prevent overflowing and/or the like when the therapeutic member 1105 is disposed in the inner volume 1186A). In some instances, the reservoir 1185 can include indicia and/or the like that can be associated with a desired fill level (i.e., a desired volume of fluid). For example, the indicia can be a fill line and/or the like. Furthermore, while not shown in FIGS. 60-62, in some embodiments, a portion of the reservoir 1185 and/or a portion of the hanging mechanism 1187 can include a relatively small hole (e.g., a pin hole or the like) that can, for example, allow steam and/or the like the vent from the inner volume 1186A. As such, the inner volume 1186A can be maintained with a pressure that is below a predetermined threshold (e.g., a pressure threshold that could damage the reservoir 1185, the therapeutic member 1105, and/or the hanging mechanism 1187, and/or that could otherwise result in an uncontrolled release of pressure such as, for example, the base 1187A of the hanging mechanism 1187 being forcibly removed from the top surface of the reservoir 1185).

Although not shown in FIGS. 60-62, in some embodiments, the arrangement of the hanging mechanism 1187 can be such that when the hanger 1189 is coupled to the therapeutic member 1105 and the base 1197A is disposed on and/or adjacent to the top surface, the coupling mechanism of the hanger 1189 and thus, the coupling mechanism of the therapeutic member 1105 (as described above) are not substantially immersed in the volume of the fluid. For example, with a predetermined volume of fluid disposed in the inner volume 1186A of the reservoir 1185 and with the therapeutic member 1105 substantially immersed therein, the coupling mechanism of the hanger 1189 (e.g., one or more snaps) and the corresponding coupling mechanism of the therapeutic member 1105 (e.g., one or more corresponding snaps) are not substantially immersed in the volume of the fluid. As such, damage to the coupling mechanism as a result of immersion in the volume of fluid can be reduced and/or eliminated.

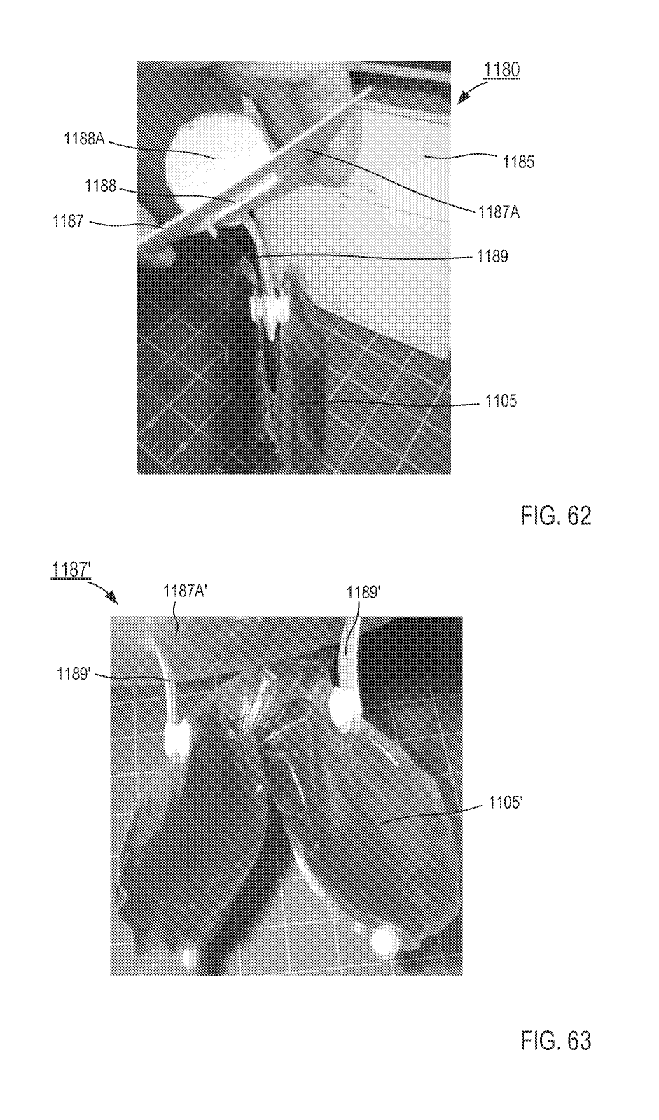

While the hanger 1189 is shown in FIG. 62 as being a single substantially elongate member that extends from the base 1187A, in other embodiments, a hanging mechanism can include one or more hangers having any suitable configuration. For example, FIG. 63 illustrates a hanging mechanism 1187′ according to an embodiment. In some instances, the hanging mechanism 1187′ can be used, for example, with the reservoir 1185 to transfer thermal energy to a therapeutic member 1105′. The hanging mechanism 1187′ includes a base 1187A′ and a hanger 1189′. The base 1187A′ can be substantially similar in form and function to the base 1187A described above. As shown in FIG. 63, the hanger 1189′ can include and/or can form two substantially elongate members that are each coupled to a different portion of the therapeutic member 1105′. In this manner, the hanging mechanism 1187′ can function substantially similar to and/or the same as the hanging mechanism 1187 described above with reference to FIGS. 60-62.

Although the hanging mechanism 1187 of FIGS. 61-62 and the hanging mechanism 1187′ of FIG. 63 include the hangers 1189 and 1189′, respectively, in other embodiments, a hanging mechanism can be coupled to a therapeutic member in any suitable manner such that when the therapeutic member is coupled thereto and is disposed in a volume of fluid, the coupling mechanism of the therapeutic member is not substantially immersed in the fluid. In other embodiments, a therapeutic member can be coupled directly to an inner surface of a reservoir in such a manner that when the reservoir is filled with a volume of fluid and the therapeutic member is substantially immersed therein, a coupling mechanism of the therapeutic member is not substantially immersed in the fluid.

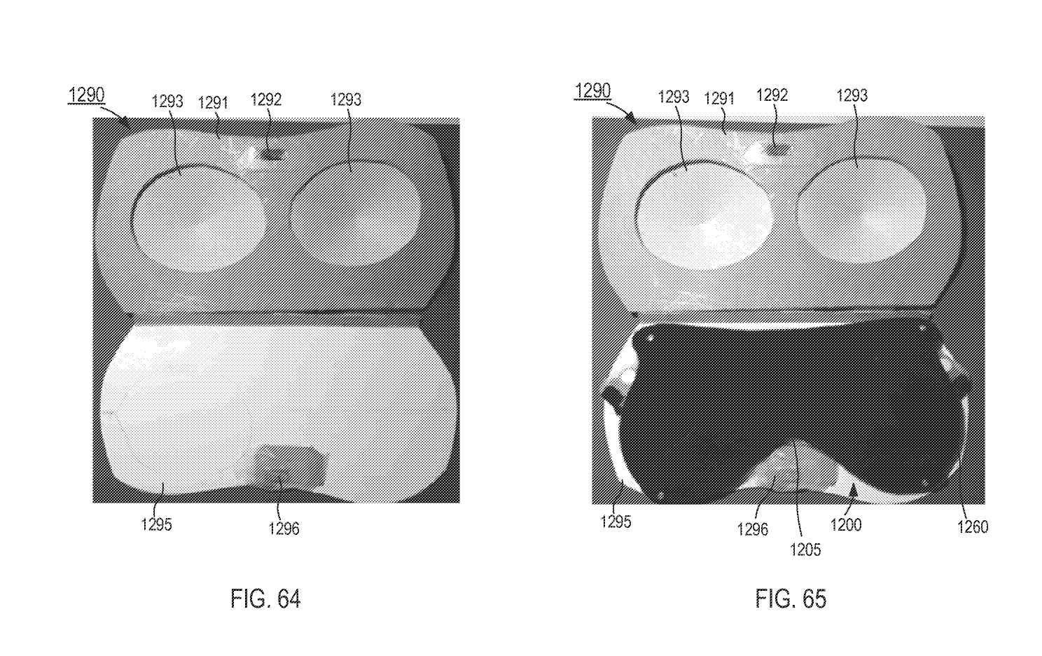

Any of the therapeutic devices and/or components thereof described herein can be disposed in any suitable packaging or the like prior to use (e.g., during shipping or the like). For example, FIGS. 64-67 illustrate a packaging 1290 configured to receive a therapeutic device 1200, according to an embodiment. In some embodiments, the packaging 1290 can be used, for example, to support and/or prevent damage to the therapeutic device 1200 during shipping and/or storage. The packaging 1290 includes a first side 1291 and a second side 1295. In some embodiments, the packaging 1290 can be formed from relatively rigid material that can resist deformation (e.g., bending, buckling, flexing, and/or otherwise reconfiguring) when exposed to an external force. The first side 1291 includes a set of engagement portions 1293 and a closure member 1292. The engagement portions 1293 can be, for example, a substantially conical structure that can be aligned with a convex portion of an insulating member included in the therapeutic device 1200 (e.g., as described above with reference to the first insulating member 260 of the therapeutic device 200), as shown in FIG. 65. Moreover, the engagement portions 1293 can be configured to form a convex structure or surface relative to an outer surface of the first side 1291 and can form a concave structure or surface relative to an inner surface of the first side 1291.

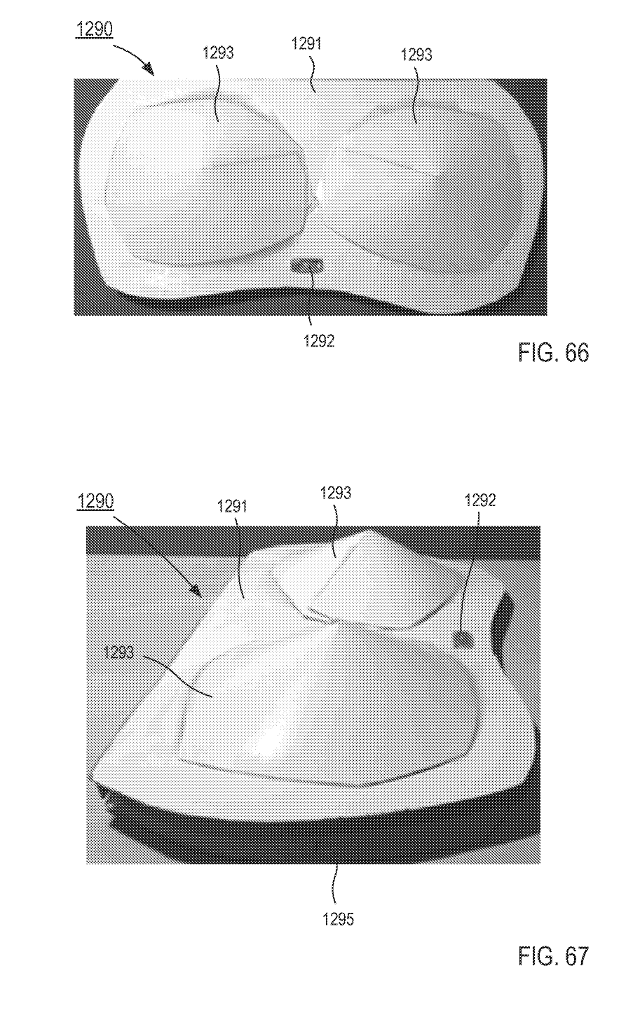

The second side 1295 of the packaging 1290 can be substantially flat and can include a closure member 1296 configured to engage the closure member 1292 of the first side 1291. In this manner, the therapeutic device 200 can be placed on the second side 1295 of the packaging 1295. As shown in FIGS. 66 and 67, the first side 1291 of the packaging 1290 can be moved relative to the second side 1291 to substantially enclose the therapeutic device 1200 therebetween. Moreover, the first side 1291 can be moved such that the engagement portions 1293 each receive a portion of the therapeutic device 1200. Expanding further, the substantially conical shape of the engagement portions 1293 conform and/or otherwise provide space for the convex portions of the therapeutic device 1200 (e.g., convex portions of a first lobe and second lobe of an insulating member, as described above with reference to the therapeutic device 200). In this manner, the engagement portions 1293 can substantially protect the convex portions of the therapeutic device 1200 from an external force (e.g., during shipping, storage, or the like) that can otherwise result in a deforming of the convex portions.

In some embodiments, the packaging 1290 can be configured so that when the first side 1291 and the second side 1292 of the packaging 1290 are moved toward one another so as to substantially enclose the therapeutic device 1200 therebetween, a compression force is exerted on a peripheral region of therapeutic device 1200 to sandwich the therapeutic device 1200 between the inner surfaces of sides 1291 and 1292. In contrast, the central regions of therapeutic device 1200 including the central lobes of an insulating member 1260 and a therapeutic member 1205 (e.g., similar to or the same as the insulating member 260 and the therapeutic member 205, described above) are substantially not exposed to the compression force, due, at least in part, to the convex engagement portions 1293 described above which can allow a vaulting above the convex portions of the insulating member 1260. Thus, as pressure is placed upon the peripheral regions of the therapeutic device 1200 by the packaging 1290, portions of the contents of the therapeutic member 1205 (e.g. gelatinous substances) can be displaced from the peripheral regions of therapeutic member 1205 and toward the central regions, including those regions underlying the convex portions of both the insulating member 1260 and the packaging 1293, as described above. The inflow of gel into these central regions can, for example, provide further structural support underneath the convex portions of the lobes of the first insulating member 1260, thus improving the stability and shaping of the convex portions of the insulating member 1260 during storage and/or the like. In some embodiments, the sides 1291 and 1292 of packaging 1290 can be left open. In other words, while the packaging 1290 is shown and described above as substantially fully protecting the contents (e.g., the therapeutic device 1200) prior to the opening of the packaging 1290, the packaging 1290 can be structured without such full protection. Such absence of peripherally closed side walls can, for example, reduce manufacturing costs, improve reusability, and reduce an overall space needed when the packaging 1290 is enclosed in other materials.

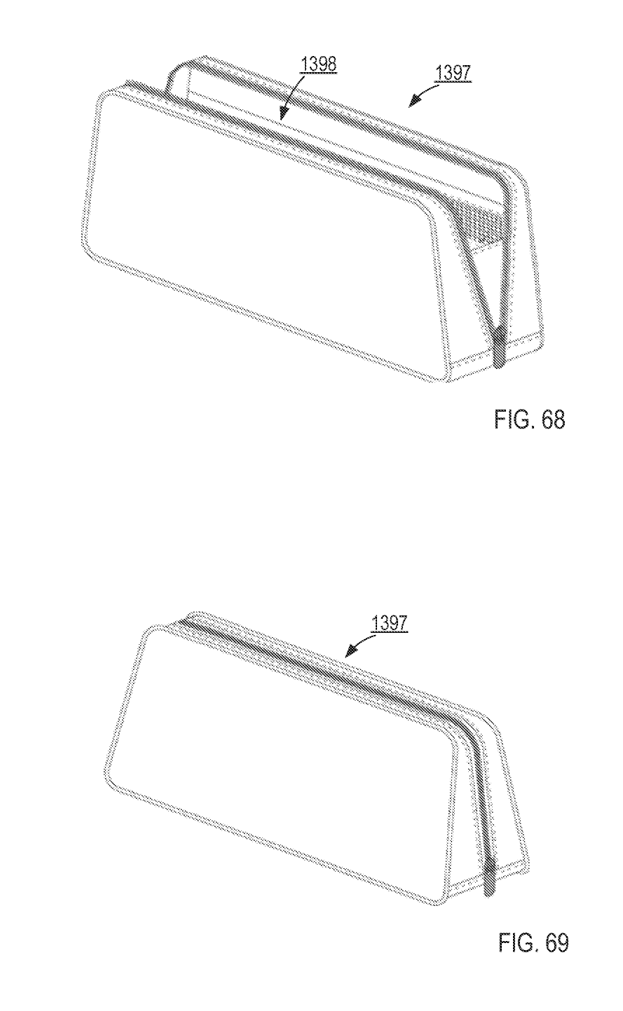

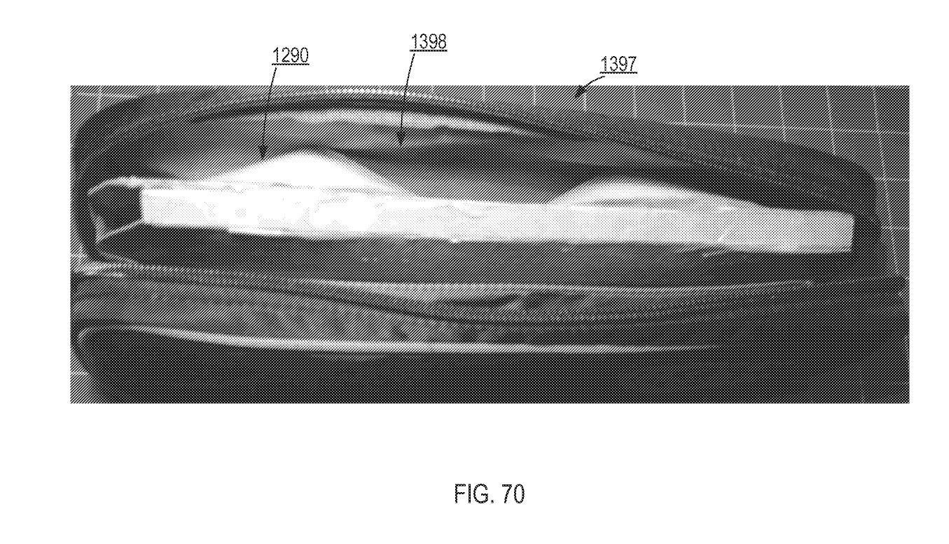

Referring now to FIGS. 68-70, any of the embodiments described herein can be stored in a case 1397 while not in use. The case 1397 can be any suitable shape, size, or configuration. For example, in some embodiments, the case 1397 can be a relatively soft pouch or the like that can define an inner volume 1398 configured to receive and store a therapeutic device. In some embodiments, the case 1397 can include a zipper closure system that can be manipulated to gain access to the inner volume 1398. In some embodiments, the case 1397 can have a base surface and a top surface, wherein a width of the base surface is greater than a width of the top surface. The wide base can allow stable upright storage of the case 1397, whether the case 1397 is stored with or without contents. As shown in FIG. 70, in some embodiments, the case 1397 can be sufficiently large such that the packaging 1290, with a therapeutic device (not shown) disposed therein, can be inserted into the inner volume 1398. Thus, the case 1397 can be configured to house and/or protect any of the therapeutic devices described herein while not in use.

Any of the therapeutic devices described herein can be configured to reduce and/or substantially diffuse a direct rearward force on the lobe of the eye. For example, the frame 210 defines the set of apertures 225 that can allow a portion of the therapeutic member 205 to extend therethrough, thereby reducing a rearward force exerted on the eye. More specifically, by selectively reducing the stiffness of the frame 210, and/or by providing apertures 225 (as described in detail above) which circumscribe a large perimeter (such as a perimeter substantially outside of an orbital rim O), a rearward force F effected by frame 210 can be distributed about a larger area of the ocular region (e.g., substantially outside of the orbital rim O), which, in turn, reduces direct rearward pressure on the eye E, as shown, for example, in FIG. 71. Thus, the frame 210 and the therapeutic member 205 can exert rearward force on the eye that is lower than a force that would otherwise be exerted by, for example, a frame and therapeutic member that did not distribute the force substantially beyond or outside of the orbital rim (e.g., a frame with greater stiffness, a frame lacking apertures, a smaller frame, or the like).

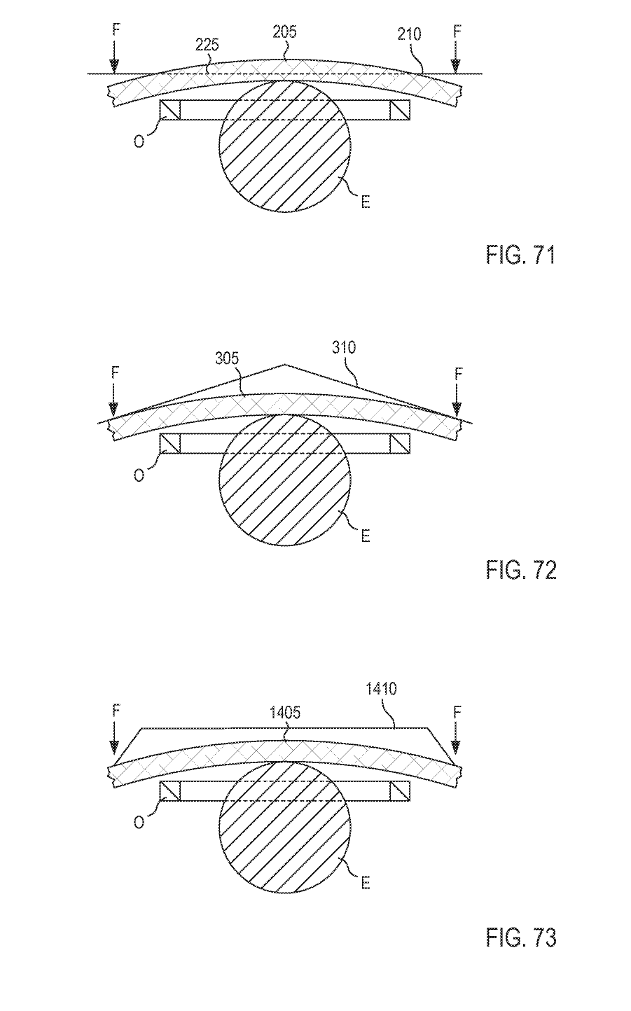

The arrangement of the frame 310 when coupled to the head of a user can similarly diffuse and/or distribute a direct rearward pressure on the eye. More specifically, by arranging the frame 310 such that the lobes have and/or form a broad convex shape, the apex of the convex-shaped lobes can be displaced anteriorly away from the globe of the eye a sufficient distance such that the therapeutic member 305 disposed therebetween is substantially not in contact with the apex. Thus, as shown in FIG. 72, the rearward force F is diffused and/or otherwise distributed on an area of the face of the user that is beyond or outside of the orbital rim O. In this manner, the frame 310 can, for example, sandwich, clamp, pin, hold, or otherwise maintain a portion of the therapeutic member 305 between a peripheral portion of the frame 310 and a portion of the face outside of the orbital rim O.

Although the lobes of the frame 310 form a substantially convex shape that is operable in distributing the rearward force F on an area outside of the orbital rim O, in other embodiments, a frame can have a lobe (or two lobes, each associated with one eye of the user) forming any suitable shape while still distributing the rearward force exerted by frame on an area of the face outside of the orbital rim. For example, as shown in FIG. 73, a frame 1410 can have a lobe with a substantially planar anterior surface and sides that extend posteriorly therefrom (e.g., a lobe having a substantially trapezoidal cross-sectional shape). Moreover, the frame 1410 can be arranged such that the sides of the lobe are in contact with a portion of the face of the user that is outside of the orbital rim O, and the anterior surface is spaced apart from the globe of the eye E a sufficient distance such that a therapeutic member 1405 is substantially not in contact with the anterior surface. Thus, the rearward force F is diffused and/or distributed on an area of the face that is beyond or outside of the orbital rim O.

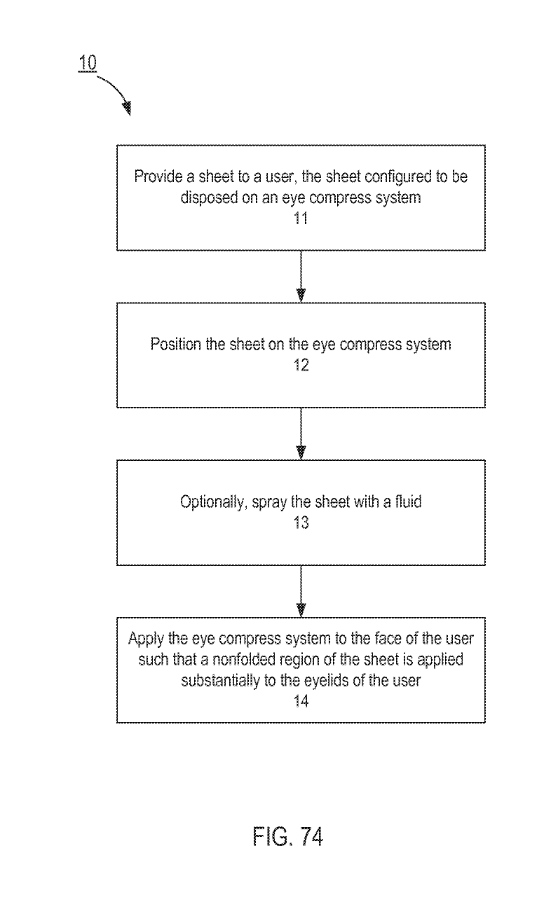

Referring now to FIG. 74, a flowchart is shown illustrating a method of using, for example, a therapeutic device, according to an embodiment. More specifically, a method 10 for using a sheet (e.g., a second insulating member) on an eye compress system (e.g., a therapeutic device) applied to an eye region of a user is shown. In some embodiments, the eye compress system can include and/or can otherwise be substantially similar to, for example, the therapeutic device 200. As such, the eye compress system can include at least a frame (e.g., such as the frame 210) coupled to a therapeutic member (e.g., such as the therapeutic member 205) configured to transfer thermal energy to the ocular region of the user.

The method 10 includes providing a sheet to a user, at 11. In some embodiments, the sheet can be, for example, any of the second insulating members described herein such as, for example, the second insulating members 270 (FIGS. 31-42), 570 (FIGS. 52 and 53), 670 (FIG. 54), and/or 770 (FIG. 55). In one embodiment, the sheet can be substantially similar to or the same as the second insulating member 270 described in detail above. In this manner, the sheet can be provided and/or otherwise disposed within a packaging such as, for example, the package 275 of FIG. 25. In some embodiments, the sheet can be disposed in the package in a substantially folded configuration. In other embodiments, the sheet can be disposed in the package in a substantially nonfolded configuration.

The sheet is positioned on the eye compress system, at 12. In some embodiments, the sheet includes at least one folded region that can substantially correspond with, for example, a set of attachment portions of a therapeutic member and/or the like. For example, in some embodiments, a therapeutic member such as the therapeutic member 205 can include a set of couplers configured to couple the therapeutic member to a frame such as the frame 210. Thus, the sheet can be disposed on a surface of the therapeutic member and can be positioned such that the folded regions substantially cover the couplers. In this manner, the increased thickness of the folded region (as described, for example, with reference to FIG. 33) can increase a user's comfort by, for example, buffering (e.g., physically, thermally, etc.) the user from the couplers.

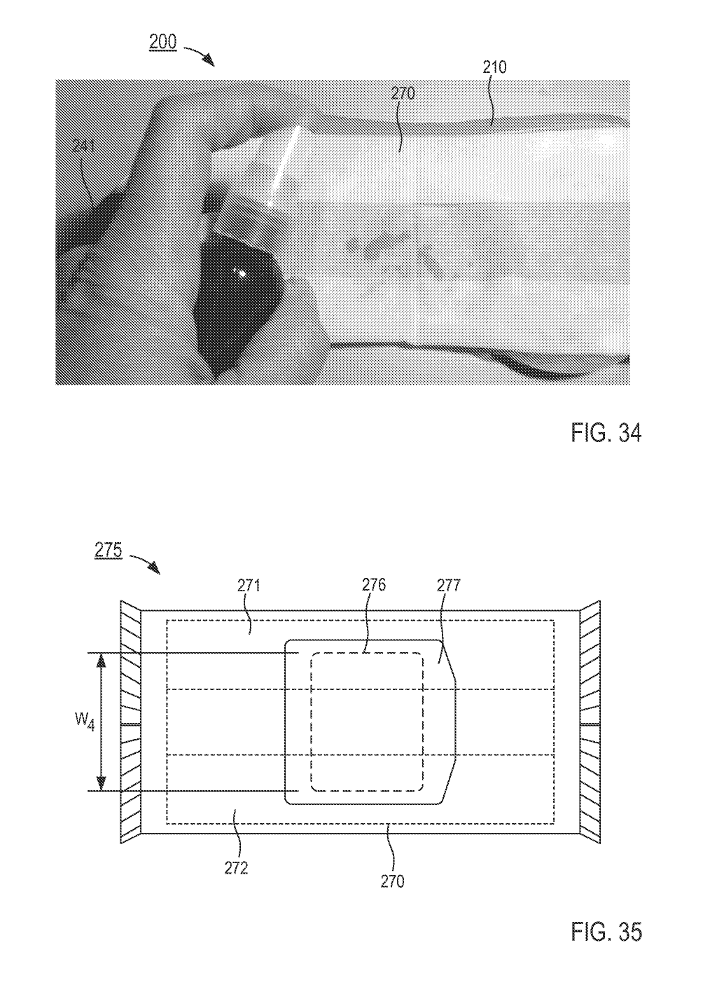

In some embodiments, the sheet can be optionally sprayed with a fluid, at 13. For example, in some embodiments, the sheet can be disposed in a package that can be configured to maintain a desired moisture content and/or level of the sheet. In some instances, a user can increase the moisture content of at least a portion of the sheet by spraying the sheet with a fluid that can be configured to, for example, enhance the transfer of thermal energy between the ocular region of the user and the eye compress system. For example, the user can spray a fluid on the sheet prior to coupling the eye compress system to his or her head. In some embodiments, the fluid can include and/or can otherwise be composed of, for example, an aqueous solvent, a facial botanical extract blend (e.g., Aloe Vera, cucumber extract, and/or the like), 1,3-Dimethylol-5,5-dimethylhydantoin (DMDM hydantoin), iodopropynyl butylcarbamate, propylene glycol, butylene glycol, and any suitable fragrance and/or aromatic agent. In this manner, the fluid can, for example, provide greater comfort to the user, by increasing the amount of moisture applied to the user's body and/or by providing an aromatic and/or other therapeutic agent, as described in detail above with reference to FIG. 34.

With the sheet having the desired moisture content, the eye compress system is applied to the face of the user such that a nonfolded region of the sheet is applied substantially to the eyelids of the user, at 14. In some embodiments, the user can couple the eye compress to his or her head in a similar manner as described above with reference to the therapeutic device 200. More particularly, in some instances, the user can manipulate the eye compress system to increase a potential thermal energy of, for example, a therapeutic member prior to applying the eye compress to his or her head. In other instances, the user can also manipulate the eye compress system to increase a potential thermal energy of, for example, a therapeutic member by manipulating the therapeutic member while the eye compress is applied to his or her head. By way of example, the user can place at least the therapeutic member in a microwave oven to add thermal energy to the therapeutic member in a similar manner as described above with reference to FIGS. 36 and 37. With the desired amount of thermal energy transferred to the therapeutic member, the user can place his or her face in contact with the sheet and can manipulate, for example, a coupling portion or the like to couple the eye compress system to his or her head such that the nonfolded region of the sheet is substantially aligned with the eyes. Thus, thermal energy can be transferred from the eye compress system to the ocular region of the user in a similar manner as described above with reference to the therapeutic device 200.

While various embodiments have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above.

Where schematics and/or embodiments described above indicate certain components arranged in certain orientations or positions, the arrangement of components may be modified. Similarly, where methods and/or events described above indicate certain events and/or procedures occurring in certain order, the ordering of certain events and/or procedures may be modified. While the embodiments have been particularly shown and described, it will be understood that various changes in form and details may be made.

Although various embodiments have been described as having particular features and/or combinations of components, other embodiments are possible having a combination of any features and/or components from any of embodiments as discussed above.

1. An apparatus, comprising:
a deformable frame positionable upon an eye region of a human user, the deformable frame including a posterior surface and an anterior surface, and a first frame section joined to a second frame section at a central bridge, the posterior surface including an attachment configured to be coupled to a thermally-adjustable therapeutic member; and
a strap assembly, the strap assembly including a first V-shaped subassembly and a second V-shaped subassembly, the first V-shaped subassembly including a first superior coupling member having an elasticity and a first inferior member having an elasticity, the first superior coupling member having an inner end portion configured to be rotatably coupled to an upper lateral portion of the first frame section, and an outer end portion, the first inferior member having an inner end portion configured to be coupled to a lower lateral portion of the first frame section and an outer end portion configured to be coupled to a first location, the first location being disposed on the first superior coupling member, the first superior coupling member and the first inferior member forming a first apex located at the first location, the elasticity of the first inferior member being substantially greater than the elasticity of the first superior coupling member;
the second V-shaped subassembly including a second superior coupling member having an elasticity and a second inferior member having an elasticity, the second superior coupling member having an inner end portion configured to be rotatably coupled to an upper lateral portion of the second frame section, and an outer end portion, the second inferior member having an inner end portion configured to be coupled to a lower lateral portion of the second frame section and an outer end portion configured to be coupled to a second location, the second location being disposed on the second superior coupling member, the second superior coupling member and the second inferior member forming a second apex located at the second location; the elasticity of the second inferior member being substantially greater than the elasticity of the second superior coupling member;
the assembly further comprising a connecting strap subassembly comprising a first connecting strap having an inner end and an outer end, the inner end of the first connecting strap joined to the outer end of the first inelastic coupling member, and a second connecting strap having an inner end and an outer end, the inner end of the second elastic connecting strap joined to the outer end of the second inelastic coupling member, wherein the outer ends of the first and second connecting straps are configured to couple to one another behind the head of the user of the assembly.

2. The apparatus of claim 1, wherein the first location and the second location are
each positioned anterior to the inner ends of the first and the second connecting straps respectively, when the deformable frame is
positioned upon the eye region of the user and the connecting straps are wrapped around the head of the user.

3. The apparatus of claim 1, wherein the first frame section includes a superotemporal region having a first cross-sectional area and an inferotemporal region having a second cross-sectional area, and the second frame section includes a superotemporal region having a third cross-sectional area and an inferotemporal region having a fourth cross-sectional area, the first cross-sectional area being less than 80% of the second cross-sectional area, the third cross-sectional area being less than 80% of the fourth cross-sectional area, such that side portions of the deformable frame will undergo a greater outward or convex bending than would otherwise result in embodiments in which the superotemporal regions were not configured to have a substantially lesser cross-sectional area than the superotemporal regions.

4. The apparatus of claim 1, wherein the first frame section includes a superotemporal region having a first stiffness and an inferotemporal region having a second stiffness, and the second frame section includes a superotemporal region having a third stiffness and an inferotemporal region having a fourth stiffness, the apparatus configured such that the first stiffness and the third stiffness are substantially less than the second stiffness and the fourth stiffness.

5. The apparatus of claim 1, wherein the attachment of the posterior surface of the frame is a snap attachment, the apparatus further comprising:
the thermally-adjustable therapeutic member, the thermally-adjustable therapeutic member including a snap attachment configured to be reversibly coupled to the snap attachment on the posterior surface of the deformable frame, the snap attachment of the thermally-adjustable therapeutic member including a cap having a first diameter and a male or a female attachment portion having a second diameter, a ratio of the first diameter to the second diameter being greater than 1.2.

6. The apparatus of claim 1, wherein the attachment of the posterior surface of the deformable frame is a snap attachment, the apparatus further comprising:
the thermally-adjustable therapeutic member, the thermally-adjustable therapeutic member including a snap attachment configured to be reversibly coupled to the snap attachment on the posterior surface of the deformable frame, the snap attachment of the thermally-adjustable therapeutic member including a cap having a first diameter and a protrusion having a second diameter, a ratio of the first diameter to the second diameter being greater than 5.4.

7. The apparatus of claim 1, further comprising:
an insulation portion having a first lobe and a second lobe, the first lobe defining a slit configured to separate the first lobe into a first leaflet and a second leaflet, the first leaflet and the second leaflet each having a distal end, the second lobe defining a slit configured to separate the second lobe into a third leaflet and a fourth leaflet, the third leaflet and the fourth leaflet each having a distal end the first lobe configured to deform into a convex tented form when the distal ends of the first leaflet and the second leaflet are coupled to the deformable frame, the second lobe configured to deform into a convex tented form when the distal ends of the third leaflet and the fourth leaflet are coupled to the deformable frame.

8. The apparatus of claim 1, further comprising:
an insulation portion having a first configuration when not attached to the deformable frame and a second configuration when attached to the deformable frame, the insulation portion being substantially flat or planar when in the first configuration such that an anterior surface and a posterior surface are substantially parallel and two dimensional, the insulation portion forming two convexities when in the second configuration, each convexity substantially configured to be positioned anterior to an eye of the user when the apparatus is applied to the eye region of the user.

9. The apparatus of claim 1, wherein the deformable frame includes a first attachment point configured to attach the first superior coupling member to the upper lateral portion of the first frame section, and a second attachment point configured to attach the second superior coupling member to the upper lateral portion of the second frame section such that when the deformable frame is not positioned upon the eye region of the user, the apparatus has a first configuration wherein a first reference axis passing through the center of the first attachment point and the second attachment point defines a first coupling member angle with a second reference axis longitudinally bisecting the first superior coupling member, the apparatus further configured such that, when the apparatus is applied to the eye region, the apparatus has a second configuration having a second coupling member angle, the second coupling member angle being greater than the first coupling member angle.

10. The apparatus of claim 9, wherein, when the apparatus is in the first configuration, the first and the second connecting straps are in a substantially more inferior position than when the apparatus is in the second configuration.

11. The apparatus of claim 1, wherein the deformable frame includes an angle of a convex bend formed by a lateral portion of the deformable frame and measured along the posterior surface of the deformable frame, wherein the frame is configured to be transitioned from a first configuration to a second configuration when the apparatus is applied to the eye region of the user, and wherein the angle of the convex bend when the apparatus is in the second configuration is less than the angle of the convex bend when the apparatus is in the first configuration when the deformable frame is positioned upon the eye region of the user such that when the apparatus is in the second configuration the angle of the convex bend is less than 160 degrees.

12. The apparatus of claim 1, wherein when the apparatus is applied to the eye region of the user and the first and second connecting straps are coupled behind the head of the user, the first superior coupling member acts as lever having a fulcrum at the upper lateral portion of the first frame section and exerts a force on the first inferior member in a superoposterior direction, and the second superior coupling member acts as a lever having a fulcrum at the upper lateral portion of the second frame section and exerts a force on the second inferior member in a superoposterior direction.

13. The apparatus of claim 1, wherein the first and the second connecting straps are substantially elastic, the first connecting strap having a first coupling portion and a first distal portion, the first coupling portion configured to be coupled to the outer end portion of the first superior coupling member, the first coupling portion having a first width, the first distal portion having a second width, the second connecting strap having a second coupling portion and a second distal portion, the second coupling portion configured to be coupled to the outer end portion of the second superior coupling member, the second coupling portion having a third width; the second distal portion having a fourth width, the second width being at least 30% greater than the first width and the fourth width being at least 30% greater than the third width, the outer end portion of the first superior coupling member being positioned anterior to a midpoint of a first ear of the user and the outer end portion of the second superior coupling member being positioned anterior to a midpoint of a second ear of the user when the deformable frame is positioned upon the eye region of the user.

14. The apparatus of claim 1 wherein the connecting strap subassembly further comprises:
a first closure member having a protrusion and a second closure member having a protrusion, the first connecting strap and the second connecting strap each defining a set of openings, the first connecting strap configured to be passed through a slot defined by the first superior coupling member and configured to be folded about a portion of the first superior coupling member such that a first opening and a second opening from the set of openings of the first connecting strap are substantially aligned coaxially and configured to receive a protrusion from the protrusion of the first closure member, the second connecting strap configured to be passed through a slot defined by the second superior coupling member and configured to be folded about a portion of the second superior coupling member such that a first opening and a second opening from the set of openings of the second connecting strap are substantially aligned coaxially and configured to receive a protrusion from the protrusion of the second closure member.

15. A method, comprising the steps of:
manufacturing a plurality of moist disposable nonwoven fabric sheets, each sheet having an upper fold defining an upper folded region extending along the length of the sheet, a lower fold defining a lower folded region extending along the length of the sheet, and a region between the upper folded region and the lower folded region, each moist disposable nonwoven fabric sheet configured for use on a therapeutic device having a thermal compress assembly, the sheet defining a perimeter having a height of at least 10 cm and a width of at least 18 cm, the region between the upper folded region and the lower folded region containing a single ply of sheet material, the height of the region between the upper folded region and the lower folded region being at least 20% of the height of the perimeter; and
placing the plurality of moist disposable fibrous nonwoven fabric sheets in a stack configured such that neither of the two fold regions of any one sheet within the stack interlocks with either of the fold regions of any other sheet within the stack; and configuring the stack within a package having a surface defining an opening, the opening configured to expose an edge of the upper folded region or the lower folded region of the topmost sheet in the stack.
